(19) United States
(12) Patent Application Publication
(10) Pub. No.: US 2006/0099704 A1
(43) Pub. Date: May 11, 2006

(54) Method for providing protein microarrays

(76) Inventors: Paul F. Predki, Carlsbad, CA (US); Barry Schweitzer, Cheshire, CT (US)

Correspondence Address: CASTELLANO MALM FERRARIO & BUCK PLLC, 2121 K STREET, NW, SUITE 800, WASHINGTON, DC, 20037, US

(21) Appl. No.: 11/183,295

(22) Filed: Jul. 14, 2005

Related U.S. Application Data
(60) Provisional application No. 60/588,158, filed on Jul. 14, 2004.
(60) Provisional application No. 60/591,541, filed on Jul. 26, 2004.
(60) Provisional application No. 60/591,827, filed on Jul. 27, 2004.
(60) Provisional application No. 60/592,239, filed on Jul. 28, 2004.
(60) Provisional application No. 60/653,586, filed on Feb. 15, 2005.

(51) Int. Cl. G06Q 99/00; C12M 1/34

(52) U.S. Cl. 435/287.2; 705/1

(57) ABSTRACT

The present application relates to methods for providing a protein microarray product and related products and services to a customer, methods, kits, and systems for labeling a probe for a protein microarray, and methods for determining protein concentrations using a protein microarray. The methods for providing a protein micorarray can include, in certain aspects, a computer function for performing some of the steps of the methods. Methods and kits for labeling a probe can include a control array that includes a molecule that binds to a label of a probe.

### RELATED APPLICATIONS

The present application claims benefit under 35 U.S.C. §119(e) of U.S. patent application Nos. 60/588,158 filed Jul. 14, 2004, 60/591,541 filed Jul. 26, 2004, 60/591,827 filed Jul. 27, 2004 60/592,239 filed Jul. 28, 2004 and 60/653,586 filed Feb. 15, 2005, all of which are incorporated herein by reference in their entireties.

### FIELD OF THE INVENTION

The present invention relates to biomolecular analysis and biotechnology products, and more particularly to biomolecular analysis and biotechnology products related to biomolecular arrays.

### BACKGROUND OF THE INVENTION

Many currently available drugs were designed without the benefit of using the intended druggable targets and structurally-related proteins, and show undesirable, or sometimes unacceptable, side effects. It is generally believed that the poor side effect profiles of currently available drugs often stem from the interaction of these drugs with (sometimes multiple) family members of the target molecule. Each family member may be involved in a physiological function distinct from the other family members. More than one family member, however, may respond to a non-specific drug. As a consequence, a non-specific drug intended to exert its effects on one physiological function may in fact influence other physiological functions, thereby causing undesirable side effects. Therefore, the pharmaceutical industry is expressing an urgent need for access to sets of gene family members and the proteins encoded by these gene family members.

Further, a major theme of pharmaceutical and biotechnology companies is to improve their lead compound selection process at the earliest stages of drug development. If these attempts are successful, those drug candidates that enter the clinic to treat human disease should possess much improved side effect and safety profiles. For example, drugs with undesirable or unacceptable side effects can be eliminated at the research stage, rather than at the clinical stage. Accordingly, there is a need to improve the lead compound selection process in order to reduce the costs associated with new drug development. Conducting research using arrays of biomolecules, such as proteins, and validating this research using related biotechnology products and services including, for example, bioinformatics research products and services, vastly improves the process of identifying lead compounds.

Pharmaceutical and biotechnology companies have invested significant resources in various genomics technologies developing, for example databases, gene expression platforms, etc. Further, a number of companies provide products and services related to these technologies. However, there is a need for more products that allow analysis of large numbers of proteins that are expressed by these genes. Furthermore, there is a need for more systems and tools that facilitate validation studies to confirm the results of protein array analysis. Finally, there is a need for an integrated system for providing genomic, proteomic, and bioinformatic products and services that presents a customer with a group of products customized based on the results of biomolecular array studies.

Citation or identification of any reference in this section or in any other section of this application, shall not be considered an admission that such reference is available as prior art to the present invention. Furthermore, section headers used herein are for the reader's convenience only.

### SUMMARY OF THE INVENTION

Provided herein in one embodiment, is a method for determining the binding affinity of a probe to a target protein, including contacting the probe with the target protein, wherein the target protein is immobilized on a positionally addressable protein array having an identifier; measuring the signal generated from probe bound to the target protein; retrieving information associated with the target protein from a database in which said information is associated with the identifier for the array, said information including the quantity and/or concentration (e.g., concentration of a protein in a solution in which it was spotted) of the target protein, and optionally the identity and quality information of the target protein; and determining the binding affinity of the probe to the target protein, by using at least part of said information. The binding affinity can be quantified relative to the binding affinity of another binding pair, or can be determined as an absolute value.

In another embodiment, provided herein is a method for providing a protein array product, typically a high-density protein microarray product, to a customer, including: providing the customer with access to a protein array product of a manufactured lot of protein array products; and providing the customer with access to identity and quantity information regarding the manufactured lot of protein array products. The protein array product typically comprises at least 100 different proteins, and can include at least 1000 different proteins. Typically, less than 25%, 10%, 5%, 1% or none of the proteins on the protein microarray are antibodies. The protein identity and quantity information identifies proteins on the array and provides quantitative information regarding proteins on the array. Optionally, the method can also include providing qualitative information regarding proteins on the array. The quantitative information is used to determine relative strengths of protein interactions or enzymatic modification of proteins on the protein microarray and a test protein contacted with the protein microarray.

In certain aspects, the protein identity, quantity and/or quality information is provided by a computer function, which in illustrative aspects is an Internet portal connected to a wide-area network. The Internet portal can also include a series of customized links for purchasing related products and/or services and/or a link to bioinformatics functions for analyzing protein array results, such as, for example, those provided in the ProtoArray™ Prospector™ manual, incorporated by reference in its entirety and available on the worldwide web at Invitrogen.com. The customized links can be customized based on the identification of one or more target proteins on the array.

In another embodiment, provided herein is a method for detecting labeling of a polypeptide or validating labeling of the polypeptide, including labeling the polypeptide with a first specific binding pair member of a first binding pair; analyzing the labeling by contacting the labeled polypeptide with a control microarray that includes a second specific binding pair member of the first binding pair associated with its surface; and analyzing binding of the labeled polypeptide to the second specific binding pair member. Where binding of the specific binding pair members is identified, the method then typically includes contacting a test microarray with the labeled polypeptide. Detectable binding of the polypeptide to the second specific binding pair member of the first binding pair is indicative of labeling of the polypeptide. In certain illustrative aspects, the second specific binding pair member of the first binding pair is associated with the surface of the control microarray by being bound to a control polypeptide on the control microarray.

In yet another embodiment, provided herein is a method for determining the concentration of a target protein, including:
a) providing a protein microarray including a spot of the target protein comprising a tag and a series of spots derived from solutions comprising different known concentrations of a control protein comprising the tag;
b) contacting the protein microarray with a first specific binding pair member that binds the tag;
c) determining a level of binding of the first specific binding pair member to the tag on the target polypeptide and to the different known concentrations of the control protein comprising the tag; and
d) determining the concentration of the target protein using the level of binding of the first specific binding pair member to the tag on the target polypeptide and the level of binding of the first specific binding pair member to the different known concentrations of the control protein comprising the tag. The concentration is usually determined using a cubic curve fitting method

The number of tags on the control protein and the target protein are typically known. For example the control protein and the target protein can include one tag molecule per protein molecule. Therefore, the method typically involves immobilizing a series of tagged control proteins of different known concentrations at a series of locations on a microarray to provide a series of spots of the tagged control proteins. Signals obtained for the series of tagged control protein spots after probing, for example with a fluorescently labeled antibody against the tag are used to generate a standard curve that is used to determine a concentration of one or more target polypeptides. The target polypeptides are typically spotted on the same array. In an illustrative embodiment, the tag is glutathione S-transferase.

In another embodiment, provided herein is a kit, including a test protein microarray including at least 10 different polypeptides; and a control protein microarray that is different than the test protein microarray. The control protein microarray includes a first specific binding pair member that binds to a first detectable label. In certain aspects, the test protein microarray includes at least 25, 50, 75, 100, 1000, 5000, 10,000, or 20,000 different proteins. In illustrative aspects the different proteins on the test array are related proteins, for example proteins from the same protein family and from the same organism. In certain examples, the first specific binding pair member is an antibody that binds the label, for example an antibody that binds biotin. Therefore, the control microarray on the kit is used to validate labeling of a test polypeptide before the test polypeptide is contacted with the test microarray. In one illustrative aspect, the kit includes 2 identical test protein microarrays and 2 identical control protein microarrays.

### BRIEF DESCRIPTION OF FIGURE

FIG. 1. Pph3 Interactions on Protein Arrays. Pathway generated in Pathblazer 2.0 software. To draw the interaction map, protein interaction data from GRID (http://biodata.mshri.on.ca/yeast_grid/servlet/SearchPage) were imported into the Pathblazer database. As a result, the modified database contains BIND and GRID interaction (genetic and biochemical) data. The proteins identified as Pph3 interactors, Rrd1 and Tip41(blue lettering) were detected on protein arrays by probing microarrays containing over 4000 affinity purified yeast proteins. Tip41 is also known to interact (two-hybrid) with the phosphatases Pph21 and Pph22. The interaction of Rrd2 with Pph3 is a genetic interaction [23]. The following interactions, Sit4-Rrd1, Sit4-Rrd2, Cla4-Cdc28 are supported by genetic and biochemical evidence [24]. Lines are interactions (genetic or biochemical). The arrow between Swe1 and Cdc28 represents inhibition by phosphorylation.

FIG. 2. The experimental workflow for probing Yeast ProtoArray™ with an in vitro biotinylated probe.

FIG. 3. Determination of protein yields for the yeast proteome. A. Subarray of Yeast ProtoArray™ PPI Proteome Microarray showing GST concentration gradient used to generate standard curve. B. Standard curve (blue circles) generated from the GST gradients in every subarray (red circles). C. Distribution of concentrations of the yeast proteome collection.

FIG. 4. Assessment of biotinylation of yeast calmodulin kinase using Western blot analysis. A. Western blot. Lane 1: SeeBlue® Plus2 Pre-Stained Standard (Invitrogen); Lane 2: Biotinylated Standard (200 fmoles); Lane 3: Biotinylated Standard (100 fmoles); Lane 4: Biotinylated Standard (50 fmoles); Lane 5: Biotinylated Standard (25 fmoles); Lane 6: Biotinylated Standard (12.5 fmoles); Lane 7 CaMK (25 fmoles) biotinylated at 3:1 molar ratio; Lane 8: CaMK (25 fmoles) biotinylated at 9:1 molar ratio; Lane 9: CaMK (25 fmoles) biotinylated at 27:1 molar ratio; Lane 10: BSA (25 fmoles) biotinylated at 9:1 molar ratio. B. Curve generated by densitometry quantitation of Lanes 2-6.

### DETAILED DESCRIPTION OF THE INVENTION

Incorporated by reference in their entirety, are the following documents, available on the worldwide web at Invitrogen.com, for example at Invitrogen.com/protoarray:

“Biomarker Identification Using ProtoArray™ High Density Protein Microarrays: “Profiling Auto-antibodies in Disease,” “Antibody Profiling on Invitrogen Protoarray™ High Density Protein Microarrays,”“Protein-Protein Interaction Profiling on Invitrogen Protoarray™ High Density Protein Microarrays,” “Performance Characteristics of the ProtoArray™ Yeast Protein-Protein Interaction (PPI) Proteome Microarray,” “Development and Validation of Kinase Substrate Screening on ProtoArray™ High-Density Protein Microarrays,” “ProtoArray™ Yeast Proteome Microarray nc v1.0 Manual “for detecting protein-protein interactions using a yeast protein microarray, “ProtoArray™ Yeast Proteome Microarray PPI Complete Kit Manual,” for protein-protein interactions with Anti-V5 Alexa Fluor® 647 Antibody mediated detection for protein-protein interactions with streptavidin Alexa Fluor® 647 mediated detection, “ProtoArray™ Control Protein Microarray nc v1.0 Manual” for rapid, efficient method for verification of probing and detection protocols, “ProtoArray™ Protein-Protein Interaction Application Kit Manual” for epitope-tagged proteins, or for biotinylated proteins; “ProtoArray™ Mini-Biotinylation Kit Manual” for biotinylation of as little as 20 ug of protein to probe against ProtoArray™ Microarrays, “ProtoArray™ Kinase Substrate Identification Manuals,” “ProtoArray™ Human Protein Microarray mg v1.0 Manual,” for kinase substrate identification using a human protein microarray, “ProtoArray™ Human Protein Microarray KSI Complete Kit Manual,” for kinase substrate identification using a human protein microarray, “ProtoArray™ Yeast Proteome Microarray mg v1.0 Manual for kinase substrate identification using a yeast proteome microarray,” “ProtoArray™ Yeast Proteome Microarray KSI Complete Kit Manual,” for kinase substrate identification using a yeast proteome microarray, and “ProtoArray™ Control Protein Microarray mg v1.0 Manual.”

DEFINITIONS

As used herein, the word “protein” refers to a full-length protein, a portion of a protein, or a peptide. Proteins can be produced via fragmentation of larger proteins, or chemically synthesized. Preferably, proteins are prepared by recombinant overexpression in a species such as, but not limited to, bacteria, yeast, insect cells, and mammalian cells. Proteins to be placed in a protein microarray of the invention preferably are fusion proteins, more preferably with at least one affinity tag to aid in purification and/or immobilization. In certain aspects of the invention, at least 2 tags are present on the protein, one of which can be used to aid in purification and the other can be used to aid in immobilization. In certain illustrative aspects, the tag is a His tag, a GST tag, or a biotin tag. Where the tag is a biotin tag, the tag can be associated with a protein in vitro or in vivo using commercially available reagents (Invitrogen, Carlsbad, Calif.). In aspects where the tag is associated with the protein in vitro, a Bioease tag can be used (Invitrogen, Carlsbad, Calif.).

As used herein, the term “peptide,” “oligopeptide,” and “polypeptide” refer to a sequence of contiguous amino acids linked by peptide bonds. A “polypeptide” refers to biomolecule with at least about twenty-five amino acids linked by peptide bonds. Peptides are typically less than about twenty-five amino acids in length. As used herein, the term “protein” refers to a polypeptide that can also include post-translational modifications.

As used herein, the term “protein array product” refers to a protein array that is offered for sale to a customer.

As used herein, the term “array” refers to an arrangement of entities in a pattern on a substrate. Although the pattern is typically a two-dimensional pattern, the pattern may also be a three-dimensional pattern. In a protein array, the entities are proteins.

As used herein, the term “microarray”

As used herein, the term “lot of protein array products” includes at least two protein array products that include identical polypeptides at identical addressable positions on the array, and that are produced by spotting isolated polypeptides from the same protein isolation procedure, typically from the same polypeptide solution. Typically, a lot of protein array products is produced by spotting polypeptides from an identical population of isolated polypeptide solutions onto a plurality of substrates consecutively, without printing proteins from other polypeptide solutions. Therefore, the specific proteins on each array of a lot of arrays are typically identical. For example a lot of protein array products can include 2, 3, 4, 5, 6, 7, 8, 9, 10, 15, 20, 25, 50, 100, 200, 250, 500, 1000, 2000, 2500, 5000, 10,000, or 100,000 protein array products. In an illustrative embodiment, a lot of a protein array products is 100 microarrays.

As used herein, the term “Internet portal” refers to an Internet site that is an entrance to functionalities, products and services related to protein arrays. An illustrative Internet portal is the Protoarray Application Portal or Protoarray Central available on the worldwide web at Invitrogen.com.

As used herein, the term “substrate” refers to the bulk, underlying, and core material of the arrays of a protein array product of the invention.

The term “fusion protein” refers to a protein composed of two or more polypeptides that, although typically unjoined in their native state, are joined by their respective amino and carboxyl termini through a peptide linkage to form a single continuous polypeptide. It is understood that the two or more polypeptide components can either be directly joined or indirectly joined through a peptide linker/spacer.

As used herein, the term “normal physiological condition” means conditions that are typical inside a living organism or a cell. While it is recognized that some organs or organisms provide extreme conditions, the intra-organismal and intra-cellular environment normally varies around pH 7 (i.e., from pH 6.5 to pH 7.5), contains water as the predominant solvent, and exists at a temperature above 0.degree. C. and below 50.degree. C. It will be recognized that the concentration of various salts depends on the organ, organism, cell, or cellular compartment used as a reference.

As used herein, the term “proteomics” means the study of or the characterization of either the proteome or some fraction of the proteome. The “proteome” is the total collection of the intracellular proteins of a cell or population of cells and the proteins secreted by the cell or population of cells. This characterization most typically includes measurements of the presence, and usually quantity, of the proteins that have been expressed by a cell, as well as analysis of the function, structural characteristics (such as post translational modification), and location within the cell of the proteins.

As used herein, the term “Functional proteomics” refers to the study of the functional characteristics, activity level, and structural characteristics of the protein expression products of a cell or population of cells.

As used herein, the term “genomic products and services” refers to products and services that are used to conduct research involving nucleic acids.

As used herein, the term “proteomic products and services” refers to products and services that are used to conduct research involving polypeptides.

As used herein, “clone collection” refers to two or more nucleic acid molecules, each of which comprises one or more nucleic acid sequences of interest.

As used herein, the term “customer” refers to any individual, institution, corporation, university, or organization seeking to obtain products and/or services, typically biotechnology products and/or services.

As used herein, the term “provider” refers to any individual, institution, corporation, university, or organization seeking to provide products and/or services, typically biotechnology products and/or services.

As used herein, the term “subscriber” refers to any customer having an agreement with a provider to obtain products and/or services on a recurring basis. The products and/or services can be free of charge or can be associated with payment of subscriber fess at subscription rates.

As used herein, the term “non-subscriber” refers to any customer who does not have an agreement with a provider to a subscription service, which is typically a service that provides a recurring product and/or service in exchange for information provided by the customer and optionally can include payment of a subscription fee, or purchase of other products and/or services by the provider.

As used herein, the term “host” refers to any prokaryotic or eukaryotic (e.g., mammalian, insect, yeast, plant, avian, animal, etc.) cell and/or organism that is a recipient of a replicable expression vector, cloning vector or any nucleic acid molecule. The nucleic acid molecule may contain, but is not limited to, a sequence of interest, a transcriptional regulatory sequence (such as a promoter, enhancer, repressor, and the like) and/or an origin of replication. As used herein, the terms “host,” “host cell,” “recombinant host” and “recombinant host cell” may be used interchangeably. For examples of such hosts, see Sambrook, et al., Molecular Cloning: A Laboratory Manual, Cold Spring Harbor Laboratory, Cold Spring Harbor, N.Y.

As used herein, the phrase “transcriptional regulatory sequence” refers to a functional stretch of nucleotides contained on a nucleic acid molecule, in any configuration or geometry, that act to regulate the transcription of (1) one or more nucleic acid sequences that may comprise ORFs, (e.g., two, three, four, five, seven, ten, etc.) into messenger RNA or (2) one or more nucleic acid sequences into untranslated RNA. Examples of transcriptional regulatory sequences include, but are not limited to, promoters, enhancers, repressors, operators (e.g., the tet operator), and the like.

As used herein, a promoter is an example of a transcriptional regulatory sequence, and is specifically a nucleic acid generally described as the 5′-region of a gene located proximal to the start codon or nucleic acid that encodes untranslated RNA. The transcription of an adjacent nucleic acid segment is initiated at or near the promoter. A repressible promoter's rate of transcription decreases in response to a repressing agent. An inducible promoter's rate of transcription increases in response to an inducing agent. A constitutive promoter's rate of transcription is not specifically regulated, though it can vary under the influence of general metabolic conditions.

As used herein, the term “insert” refers to a desired nucleic acid segment that is a part of a larger nucleic acid molecule. In many instances, the insert will be introduced into the larger nucleic acid molecule using techniques known to those of skill in the art, e.g., recombinational cloning, topoisomerase cloning or joining, ligation, etc.

As used herein, the phrase “target nucleic acid molecule” refers to a nucleic acid molecule that includes at least one nucleic acid sequence of interest, preferably a nucleic acid molecule that is to be acted upon using the compounds and methods of the present invention. Such target nucleic acid molecules may contain one or more (e.g., two, three, four, five, seven, ten, twelve, fifteen, twenty, thirty, fifty, etc.) sequences of interest.

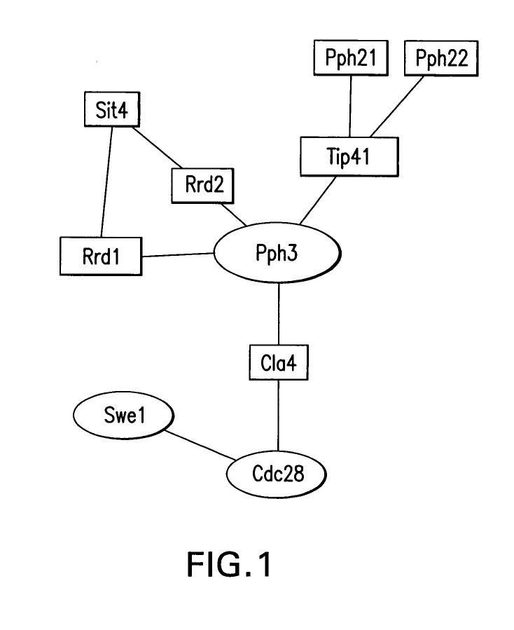

As used herein, the phrase “recognition sequence” or “recognition site” refers to a particular sequence to which a protein, chemical compound, DNA, or RNA molecule (e.g., restriction endonuclease, a topoisomerase, a modification methylase, a recombinase, etc.) recognizes and binds. In the present invention, a recognition sequence may refer to a recombination site. For example, the recognition sequence for Cre recombinase is loxP which is a 34 base pair sequence comprising two 13 base pair inverted repeats (serving as the recombinase binding sites) flanking an 8 base pair core sequence (see FIG. 1 of Sauer, B., Current Opinion in Biotechnology 5:521-527 (1994)). Other examples of recognition sequences are the attB, attP, attL, and attR sequences, which are recognized by the recombinase enzyme λ Integrase. attB is an approximately 25 base pair sequence containing two 9 base pair core-type Int binding sites and a 7 base pair overlap region. attP is an approximately 240 base pair sequence containing core-type Int binding sites and arm-type Int binding sites as well as sites for auxiliary proteins integration host factor (IHF), FIS and excisionase (Xis) (see Landy, Current Opinion in Biotechnology 3:699-707 (1993)). Such sites may also be engineered according to the present invention to enhance production of products in the methods of the invention. For example, when such engineered sites lack the P1 or H1 domains to make the recombination reactions irreversible (e.g., attR or attP), such sites may be designated attR′ or attP′ to show that the domains of these sites have been modified in some way.

As used herein, the term “vector” refers to a nucleic acid molecule (preferably DNA) that provides a useful biological or biochemical property to an insert. Examples include plasmids, phages, viruses, autonomously replicating sequences (ARS), centromeres, and other sequences that are able to replicate or be replicated in vitro or in a host cell, or to convey a desired nucleic acid segment to a desired location within a host cell. A vector can have one or more restriction endonuclease recognition sites (e.g., two, three, four, five, seven, ten, etc.) at which the sequences can be cut in a determinable fashion without loss of an essential biological function of the vector, and into which a nucleic acid fragment can be spliced in order to bring about its replication and cloning. Vectors can further provide primer sites (e.g., for PCR), transcriptional and/or translational initiation and/or regulation sites, recombinational signals, replicons, selectable markers, etc. Clearly, methods of inserting a desired nucleic acid fragment that do not require the use of recombination, transpositions or restriction enzymes (such as, but not limited to, uracil N-glycosylase (UDG) cloning of PCR fragments (U.S. Pat. Nos. 5,334,575 and 5,888,795, both of which are entirely incorporated herein by reference), T:A cloning, and the like) can also be applied to clone a fragment into a cloning vector to be used according to the present invention. The cloning vector can further contain one or more selectable markers (e.g., two, three, four, five, seven, ten, etc.) suitable for use in the identification of cells transformed with the cloning vector.

As used herein, the phrase “subcloning vector” refers to a cloning vector comprising a circular or linear nucleic acid molecule that includes, preferably, an appropriate replicon. In the present invention, the subcloning vector can also contain functional and/or regulatory elements that are desired to be incorporated into the final product to act upon or with the cloned nucleic acid insert. The subcloning vector can also contain a selectable marker (preferably DNA).

As used herein, the term “primer” refers to a single stranded or double stranded oligonucleotide that is extended by covalent bonding of nucleotide monomers during amplification or polymerization of a nucleic acid molecule (e.g., a DNA molecule). In one aspect, the primer may be a sequencing primer (for example, a universal sequencing primer). In another aspect, the primer may comprise a recombination site or portion thereof.

As used herein, the term “adapter” refers to an oligonucleotide or nucleic acid fragment or segment (preferably DNA) that comprises one or more recombination sites (or portions of such recombination sites) that can be added to a circular or linear nucleic acid molecule as well as to other nucleic acid molecules described herein. When using portions of recombination sites, the missing portion may be provided by the nucleic acid molecule. Such adapters may be added at any location within a circular or linear molecule, although the adapters are preferably added at or near one or both termini of a linear molecule. Preferably, adapters are positioned to be located on both sides (flanking) a particular nucleic acid molecule of interest. In accordance with the invention, adapters may be added to nucleic acid molecules of interest by standard recombinant techniques (e.g., restriction digest and ligation). For example, adapters may be added to a circular molecule by first digesting the molecule with an appropriate restriction enzyme, adding the adapter at the cleavage site and reforming the circular molecule that contains the adapter(s) at the site of cleavage. In other aspects, adapters may be added by homologous recombination, by integration of RNA molecules, and the like. Alternatively, adapters may be ligated directly to one or more and preferably both termini of a linear molecule thereby resulting in linear molecule(s) having adapters at one or both termini. In one aspect of the invention, adapters may be added to a population of linear molecules, (e.g., a cDNA library or genomic DNA that has been cleaved or digested) to form a population of linear molecules containing adapters at one and preferably both termini of all or substantial portion of said population.

As used herein, the phrase “adapter-primer” refers to a primer molecule that comprises one or more recombination sites (or portions of such recombination sites) that can be added to a circular or to a linear nucleic acid molecule described herein. When using portions of recombination sites, the missing portion may be provided by a nucleic acid molecule (e.g., an adapter) of the invention. Such adapter-primers may be added at any location within a circular or linear molecule, although the adapter-primers are preferably added at or near one or both termini of a linear molecule. Such adapter-primers may be used to add one or more recombination sites or portions thereof to circular or linear nucleic acid molecules in a variety of contexts and by a variety of techniques, including but not limited to amplification (e.g., PCR), ligation (e.g., enzymatic or chemical/synthetic ligation), recombination (e.g., homologous or non-homologous (illegitimate) recombination) and the like.

As used herein, the term “template” refers to a double stranded or single stranded nucleic acid molecule, all or a portion of which is to be amplified, synthesized, reverse transcribed, or sequenced. In the case of a double-stranded DNA molecule, denaturation of its strands to form a first and a second strand is preferably performed before these molecules may be amplified, synthesized or sequenced, or the double stranded molecule may be used directly as a template. For single stranded templates, a primer complementary to at least a portion of the template hybridizes under appropriate conditions and one or more polypeptides having polymerase activity (e.g., two, three, four, five, or seven DNA polymerases and/or reverse transcriptases) may then synthesize a molecule complementary to all or a portion of the template. Alternatively, for double stranded templates, one or more transcriptional regulatory sequences (e.g., two, three, four, five, seven or more promoters) may be used in combination with one or more polymerases to make nucleic acid molecules complementary to all or a portion of the template. The newly synthesized molecule, according to the invention, may be of equal or shorter length compared to the original template. Mismatch incorporation or strand slippage during the synthesis or extension of the newly synthesized molecule may result in one or a number of mismatched base pairs. Thus, the synthesized molecule need not be exactly complementary to the template. Additionally, a population of nucleic acid templates may be used during synthesis or amplification to produce a population of nucleic acid molecules typically representative of the original template population.

As used herein, the term “incorporating” means becoming a part of a nucleic acid (e.g., DNA) molecule or primer.

As used herein, the term “nucleic acid library” refers to a collection of nucleic acid molecules (circular or linear). In one embodiment, a library can include a plurality of nucleic acid molecules (e.g., two, three, four, five, seven, ten, twelve, fifteen, twenty, thirty, fifty, one hundred, two hundred, five hundred one thousand, five thousand, or more), that may or may not be from a common source organism, organ, tissue, or cell. In another embodiment, a library is representative of all or a portion or a significant portion of the nucleic acid content of an organism (a “genomic” library), or a set of nucleic acid molecules representative of all or a portion or a significant portion of the expressed nucleic acid molecules (a cDNA library or segments derived therefrom) in a cell, tissue, organ or organism. A library can also include nucleic acid molecules having random sequences made by de novo synthesis, mutagenesis of one or more nucleic acid molecules, and the like. Such libraries may or may not be contained in one or more vectors (e.g., two, three, four, five, seven, ten, twelve, fifteen, twenty, thirty, fifty, etc.). In some embodiments, a library may be “normalized” library (i.e., a library of cloned nucleic acid molecules from which each member nucleic acid molecule can be isolated with approximately equivalent probability).

As used herein, the term “normalized” or “normalized library” means a nucleic acid library that has been manipulated, preferably using the methods of the invention, to reduce the relative variation in abundance among member nucleic acid molecules in the library to a range of no greater than about 25-fold, no greater than about 20-fold, no greater than about 15-fold, no greater than about 10-fold, no greater than about 7-fold, no greater than about 6-fold, no greater than about 5-fold, no greater than about 4-fold, no greater than about 3-fold or no greater than about 2-fold.

As used herein, the term “amplification” refers to any in vitro method for increasing the number of copies of a nucleic acid molecule with the use of one or more polypeptides having polymerase activity (e.g., one, two, three, four or more nucleic acid polymerases or reverse transcriptases). Nucleic acid amplification results in the incorporation of nucleotides into a DNA and/or RNA molecule or primer thereby forming a new nucleic acid molecule complementary to a template. The formed nucleic acid molecule and its template can be used as templates to synthesize additional nucleic acid molecules. As used herein, one amplification reaction may consist of many rounds of nucleic acid replication. DNA amplification reactions include, for example, polymerase chain reaction (PCR). One PCR reaction may consist of 5 to 100 cycles of denaturation and synthesis of a DNA molecule.

As used herein, the term “nucleotide” refers to a base-sugar-phosphate combination. Nucleotides are monomeric units of a nucleic acid molecule (DNA and RNA). The term nucleotide includes ribonucleoside triphosphates ATP, UTP, CTG, GTP and deoxyribonucleoside triphosphates such as dATP, dCTP, dITP, dUTP, dGTP, dTTP, or derivatives thereof. Such derivatives include, for example, [α-S]dATP, 7-deaza-dGTP and 7-deaza-dATP. The term nucleotide as used herein also refers to dideoxyribonucleoside triphosphates (ddNTPs) and their derivatives. Illustrated examples of dideoxyribonucleoside triphosphates include, but are not limited to, ddATP, ddCTP, ddGTP, ddITP, and ddTTP. According to the present invention, a “nucleotide” may be unlabeled or detectably labeled by well known techniques. Detectable labels include, for example, radioactive isotopes, fluorescent labels, chemiluminescent labels, bioluminescent labels and enzyme labels.

As used herein, the phrase “nucleic acid molecule” refers to a sequence of contiguous nucleotides (riboNTPs, dNTPs, ddNTPs, or combinations thereof) of any length. A nucleic acid molecule can encode a full-length polypeptide or a fragment of any length thereof, or can be non-coding. As used herein, the terms “nucleic acid molecule” and “polynucleotide” can be used interchangeably and include both RNA and DNA.

As used herein, the term “oligonucleotide” refers to a synthetic or natural molecule comprising a covalently linked sequence of nucleotides that are joined by a phosphodiester bond between the 3′ position of the pentose of one nucleotide and the 5′ position of the pentose of the adjacent nucleotide.

As used herein, an open reading frame or ORF refers to a sequence of nucleotides that codes for a contiguous sequence of amino acids. ORFs of the invention may be constructed to code for the amino acids of a polypeptide of interest from the N-termius of the polypeptide (typically a methionine encoded by a sequence that is transcribed as AUG) to the C-terminus of the polypeptide. ORFs of the invention include sequences that encode a contiguous sequence of amino acids with no intervening sequences (e.g., an ORF from a cDNA) as well as ORFs that comprise one or more intervening sequences (e.g., introns) that may be processed from an mRNA containing them (e.g., by splicing) when an mRNA containing the ORF is transcribed in a suitable host cell. ORFs of the invention also comprise splice variants of ORFs containing intervening sequences.

ORFs can optionally be provided with one or more sequences that function as stop codons (e.g., contain nucleotides that are transcribed as UAG, an amber stop codon, UGA, an opal stop codon, and/or UAA, an ochre stop codon). When present, a stop codon can be provided after the codon encoding the C-terminus of a polypeptide of interest (e.g., after the last amino acid of the polypeptide) and/or may be located within the coding sequence of the polypeptide of interest. When located after the C-terminus of the polypeptide of interest, a stop codon may be immediately adjacent to the codon encoding the last amino acid of the polypeptide or there may be one or more codons (e.g., one, two, three, four, five, ten, twenty, etc) between the codon encoding the last amino acid of the polypeptide of interest and the stop codon. A nucleic acid molecule containing an ORF may be provided with a stop codon upstream of the initiation codon (e.g., an AUG codon) of the ORF. When located upstream of the initiation codon of the polypeptide of interest, a stop codon may be immediately adjacent to the initiation codon or there may be one or more codons (e.g., one, two, three, four, five, ten, twenty, etc) between the initiation codon and the stop codon.

As used herein, the word “interactor” refers to a protein on a protein microarray that interacts with a probe.

As used herein, the word “probe” refers to any chemical reagent such as, but not limited to, a protein, nucleic acid (e.g., DNA, RNA, oligonucleotide, polynucleotide), small molecule, substrate, inhibitor, drug or drug candidate, receptor, antigen, hormone, steroid, lipid, phospholipid, liposome, antibody, cofactor, cytokine, glutathione, immunoglobulin domain, carbohydrate, maltose, nickel, dihydrotrypsin, calmodulin, biotin, lectin, and heavy metal, that can be applied to a protein array of the invention to assay for interaction with a protein of the array.

As used herein, the phrase “recombination proteins” includes excisive or integrative proteins, enzymes, co-factors or associated proteins that are involved in recombination reactions involving one or more recombination sites (e.g., two, three, four, five, seven, ten, twelve, fifteen, twenty, thirty, fifty, etc.), which may be wild-type proteins (see Landy, Current Opinion in Biotechnology 3:699-707 (1993)), or mutants, derivatives (e.g., fusion proteins containing the recombination protein sequences or fragments thereof), fragments, and variants thereof. Examples of recombination proteins include Cre, Int, IHF, Xis, Flp, Fis, Hin, Gin, ΦC31, Cin, Tn3 resolvase, TndX, XerC, XerD, TnpX, Hjc, Gin, SpCCE1, and ParA.

As used herein, the term “recombinases” refers to a protein that catalyzes strand cleavage and re-ligation in a recombination reaction. Site-specific recombinases are proteins that are present in many organisms (e.g., viruses and bacteria) and have been characterized as having both endonuclease and ligase properties. These recombinases (along with associated proteins in some cases) recognize specific sequences of bases in a nucleic acid molecule and exchange the nucleic acid segments flanking those sequences. The recombinases and associated proteins are collectively referred to as “recombination proteins” (see, e.g., Landy, A., Current Opinion in Biotechnology 3:699-707 (1993)).

Numerous recombination systems from various organisms have been described. See, e.g., Hoess, et al., Nucleic Acids Research 14(6):2287 (1986); Abremski, et al., J. Biol. Chem. 261(1):391 (1986); Campbell, J. Bacteriol. 174(23):7495 (1992); Qian, et al., J. Biol. Chem. 267(11):7794 (1992); Araki, et al., J. Mol. Biol. 225(1):25 (1992); Maeser and Kahnmann, Mol. Gen. Genet. 230:170-176 (1991); Esposito, et al., Nucl. Acids Res. 25(18):3605 (1997). Many of these belong to the integrase family of recombinases (Argos, et al., EMBO J. 5:433-440 (1986); Voziyanov, et al., Nucl. Acids Res. 27:930 (1999)). Perhaps the best studied of these are the Integrase/att system from bacteriophage λ (Landy, A. Current Opinions in Genetics and Devel. 3:699-707 (1993)), the Cre/loxP system from bacteriophage P1 (Hoess and Abremski (1990) In Nucleic Acids and Molecular Biology, vol. 4. Eds.: Eckstein and Lilley, Berlin-Heidelberg: Springer-Verlag; pp. 90-109), and the FLP/FRT system from the Saccharomyces cerevisiae 2μ circle plasmid (Broach, et al., Cell 29:227-234 (1982)).

A used herein, the phrase “recombination site” refers to a recognition sequence on a nucleic acid molecule that participates in an integration/recombination reaction by recombination proteins. Recombination sites are discrete sections or segments of nucleic acid on the participating nucleic acid molecules that are recognized and bound by a site-specific recombination protein during the initial stages of integration or recombination. For example, the recombination site for Cre recombinase is loxP, which is a 34 base pair sequence comprised of two 13 base pair inverted repeats (serving as the recombinase binding sites) flanking an 8 base pair core sequence (see FIG. 1 of Sauer, B., Curr. Opin. Biotech. 5:521-527 (1994)). Other examples of recombination sites include the attB, attP, attL, and attR sequences described in U.S. provisional patent applications 60/136,744, filed May 28, 1999, and 60/188,000, filed Mar. 9, 2000, and in co-pending U.S. patent application Ser. Nos. 09/517,466 and 09/732,91—all of which are specifically incorporated herein by reference—and mutants, fragments, variants and derivatives thereof, which are recognized by the recombination protein λ Int and by the auxiliary proteins integration host factor (IHF), FIS and excisionase (Xis) (see Landy, Curr. Opin. Biotech. 3:699-707 (1993)).

Mutating specific residues in the core region of the att site can generate a large number of different att sites. As with the att1 and att2 sites utilized in GATEWAY™, each additional mutation potentially creates a novel att site with unique specificity that will recombine only with its cognate partner att site bearing the same mutation and will not cross-react with any other mutant or wild-type att site. Novel mutated att sites (e.g., attB 1-10, attP 1-10, attR 1-10 and attL 1-10) are described in previous patent application Ser. No. 09/517,466, filed Mar. 2, 2000, which is specifically incorporated herein by reference. Other recombination sites having unique specificity (i.e., a first site will recombine with its corresponding site and will not recombine or not substantially recombine with a second site having a different specificity) may be used to practice the present invention. Examples of suitable recombination sites include, but are not limited to, loxP sites; loxP site mutants, variants or derivatives such as loxP511 (see U.S. Pat. No. 5,851,808); frt sites; frt site mutants, variants or derivatives; dif sites; dif site mutants, variants or derivatives; psi sites; psi site mutants, variants or derivatives; cer sites; and cer site mutants, variants or derivatives.

Recombination sites may be added to molecules by any number of known methods. For example, recombination sites can be added to nucleic acid molecules by blunt end ligation, PCR performed with fully or partially random primers, or inserting the nucleic acid molecules into a vector using a restriction site flanked by recombination sites.

As used herein, the phrase “recombinational cloning” refers to a method whereby segments of nucleic acid molecules or populations of such molecules are exchanged, inserted, replaced, substituted or modified, in vitro or in vivo. Preferably, such cloning method is an in vitro method.

Suitable recombinational cloning systems that utilize recombination at defined recombination sites have been previously described in U.S. Pat. No. 5,888,732, U.S. Pat. No. 6,143,557, U.S. Pat. No. 6,171,861, U.S. Pat. No. 6,270,969, and U.S. Pat. No. 6,277,608, and in pending U.S. application Ser. No. 09/517,466, and in published United States application no. 20020007051, (each of which is fully incorporated herein by reference), all assigned to the Invitrogen Corporation, Carlsbad, Calif. In brief, the GATEWAY™ Cloning System described in these patents utilizes vectors that contain at least one recombination site to clone desired nucleic acid molecules in vivo or in vitro. In some embodiments, the system utilizes vectors that contain at least two different site-specific recombination sites that may be based on the bacteriophage lambda system (e.g., att1 and att2) that are mutated from the wild-type (att0) sites. Each mutated site has a unique specificity for its cognate partner att site (i.e., its binding partner recombination site) of the same type (for example attB1 with attP1, or attL1 with attR1) and will not cross-react with recombination sites of the other mutant type or with the wild-type att0 site. Different site specificities allow directional cloning or linkage of desired molecules thus providing desired orientation of the cloned molecules. Nucleic acid fragments flanked by recombination sites are cloned and subcloned using the GATEWAY™ system by replacing a selectable marker (for example, ccdB) flanked by att sites on the recipient plasmid molecule, sometimes termed the Destination Vector. Desired clones are then selected by transformation of a ccdB sensitive host strain and positive selection for a marker on the recipient molecule. Similar strategies for negative selection (e.g., use of toxic genes) can be used in other organisms such as thymidine kinase (TK) in mammals and insects.

As used herein, the term “topoisomerase recognition site” means a defined nucleotide sequence that is recognized and bound by a site specific topoisomerase. For example, the nucleotide sequence 5′-(C/T)CCTT-3′ is a topoisomerase recognition site that is bound specifically by most poxvirus topoisomerases, including vaccinia virus DNA topoisomerase I, which then can cleave the strand after the 3′-most thymidine of the recognition site to produce a nucleotide sequence comprising 5′-(C/T)CCTT-PO₄-TOPO, i.e., a complex of the topoisomerase covalently bound to the 3′ phosphate through a tyrosine residue in the topoisomerase (see, Shuman, J. Biol. Chem. 266:11372-11379, 1991; Sekiguchi and Shuman, Nucl. Acids Res. 22:5360-5365, 1994; each of which is incorporated herein by reference; see, also, U.S. Pat. No. 5,766,891; PCT/US95/16099; and PCT/US98/12372). In comparison, the nucleotide sequence 5′-GCAACTT-3′ is the topoisomerase recognition site for type IA E. coli topoisomerase III.

In certain embodiments of the invention, the concentration of a protein at a locus on an array refers to the concentration of the protein in solution when the protein was initially deposited at that locus on the array.

The term “database” as used herein refers to any collection of data. In certain embodiments, a database is in computer-readable form. In certain embodiments, a computer-readable database is in ASCII format. In an illustrative embodiment, a database is in the form of a Word document, an Excel spreadsheet. The database can also be a database program, such as a relational database, of which many are well known in the art, such as Microsoft Access or Microsoft SQL Server.

As used herein, the term “array” refers to an arrangement of entities in a pattern on a substrate. Although the pattern is typically a two-dimensional pattern, the pattern may also be a three-dimensional pattern. In a protein array, the entities are proteins. In certain embodiments, the array can be a microarray or a nanoarray. A “nanoarray,” is an array in which separate entities are separated by 0.1 nm to 10 um, for example from 1 nm to 1 um. A microarray is an array in which separate entities are separated by more than 1 um and the density of entities on the array is at least 100/cm².

The term “protein microarray” as used herein refers to a protein microarray or a protein nanoarray.

The term “protein chip” is used in the present application synonymously with protein microarray.

Methods for Determining Binding Affinity.

The invention provides a method for determining the binding affinity of a probe to a target protein, including contacting the probe with the target protein, wherein the target protein is immobilized on a positionally addressable protein array having an identifier; measuring the signal generated from probe bound to the target protein; retrieving information associated with the target protein from a database in which said information is associated with the identifier for the array, said information including the identity, quantity and/or quality information of the target protein; and determining the binding affinity of the probe to the target protein, using at least part of said information.

In certain embodiments, a positionally addressable protein array is screened with a labeled probe under conditions conducive to the binding between the probe and a protein. Typically, the positionally addressable protein array is associated with an identifier. The identifier is associated with information such as identity, location and concentration of the proteins on the positionally addressable protein array. Binding of the labeled probe to a protein on the array can be detected by any method known to the skilled artisan. In certain specific embodiments, the label itself provides a signal (e.g., if the label is a fluorophore) or the label is capable of catalyzing a reaction that generates a detectable signal. In other specific embodiments, the label is detected using a detectably labeled molecule that binds to the label. The detectable label of the molecule that binds to the label can be a fluorophore or a molecule that catalyzes a reaction, wherein the reaction generates a detectable signal (e.g., a colorimetric reaction). In an illustrative embodiment, the label is biotin and the biotin is detected using streptavidin, wherein the streptavidin is labeled with a fluorophore. Binding of the labeled probe to a protein on the array generates a detectable signal. Based on the location of the signal, the identity of the protein, i.e., the binding partner, can be obtained. Information regarding the identity and quantity of proteins on the array can be retrieved using the identifier that is associated with the microarray, for example by providing access to a database containing the information. In certain embodiments, such information is retrieved through the Internet from a remote location. In specific embodiments, based on the concentration of the binding partner on the microarray, the amount of probe used and the intensity of signal generated by the probe bound to the binding partner, the affinity of the probe to the binding partner can be calculated. In certain, even more specific embodiments, information about the efficiency of the labeling of the probe is factored into the calculation of the binding affinity of the probe to the binding partner.

In certain specific embodiments, the labeled probe is used to identify binding partners of the probe and/or to determine the affinity of the probe to a binding partner. In certain specific embodiments, the labeled probe is used to screen a protein array to identify a binding partner and/or to determine the affinity of the probe to the binding partner.

In a specific embodiment, to determine the affinity of the probe to the binding partner, the labeled probe is used to screen a positionally addressable protein microarray. In certain specific embodiments, the probe is labeled and separated into several aliquots. One aliquot of the labeled probe is tested for the efficiency of the labeling reaction using a control array (as described in the section “METHOD FOR VALIDATING LABELING OF A PROBE”). Another aliquot of the labeled probe is used to identify binding partners on an array, e.g., a positionally addressable protein microarray, and/or determine the binding affinity of the probe to a binding partner on the array. Without being bound by theory, the proportion of labeled probe in the aliquot of labeled probe is factored into the calculation of how much probe is bound to the binding partner. In specific embodiments, the labeled probe is affinity purified using a molecule that specifically binds to the label.

In certain aspects, the positionally addressable protein array comprises at least 100, 200, 250, 500, 1000, 2500, or 5000 proteins/cm². Furthermore, the positionally addressable protein array can include, in certain illustrative examples, at least 100, 250, 500, 1000, 2500, 5000, or 10,000 different proteins, including different polymorphic variants, which can be from the same organism.

In a related embodiment, provided herein is a method for determining a strength of interaction between a target protein on a protein array and a probe or a strength of enzymatic modification of the target protein on the protein array by the probe, comprising
a) performing an assay by contacting proteins on a protein array with the probe to identify a positive signal on the array;
b) obtaining information regarding the identity and quantity of proteins on the protein array from a supplier of the protein array;
c) identifying the protein associated with the positive signal using the information regarding the identity and quantity of proteins on the protein array; and
d) identifying the relative strength of interaction or enzymatic modification of the target protein on the protein array using the information regarding the identity and quantity of proteins on the protein array.
   Method for Providing a Protein Array Product to a Customer

In another embodiment, the present invention is based, in part, on the availability of an Internet portal that provides access to lot-specific protein microarray concentration and identity information, provides access to tools for analyzing microarray data, and/or provides access to customized lists and purchasing functions of products that are related to target proteins that are identified on a protein microarray. Therefore, the present invention facilitates analysis of protein microarray experimental results and facilitates follow-up experiments from protein microarray experiments, which may require large numbers of reagents and services based on large number of target proteins identified using protein microarrays. Accordingly, provided herein is a method for providing a protein microarray product to a customer, that includes providing the customer with access to a protein array product, typically a high density protein microarray product that typically includes at least 100, 250, 500, 1000, 2000, 2500, 3000, 4000, 5000, 7500, or 10000 different proteins of a manufactured lot of protein microarray products; and providing the customer with protein identity and quantitative information regarding the at least 100, 250, 500, 1000, 2000, 2500. 3000, 4000, 5000, 7500, or 10000 different proteins on each protein microarray product of the manufactured lot of protein microarray products, thereby providing the protein microarray product to the customer. The method can further include providing access to a computer function for obtaining the identity and quantitative information of proteins on the protein microarray product based on an identifier of the protein microarray product or the manufactured lot of protein microarray products. In illustrative examples, the computer function further provides access to the customer, to a purchasing function for identifying one or more target proteins on the protein microarray product, and for purchasing one or more related products and/or services related to the identified target proteins. The purchasing function presents the customer with access to a customized series of computer links to the related products and/or services based on the identified one or more target proteins. The one or more target proteins can be identified, for example, based on one or more positive signals identified by an image analysis function, which in certain aspects is provided by the computer function. The computer function in illustrative embodiments, is an Internet portal that is provided over a wide area network to the customer by a provider of the protein microarray product. In certain aspects, less than 25%, 10%, 5%, 1% or none of the proteins on the protein microarray are antibodies. The quantitative information regarding proteins on the array in certain illustrative examples includes the concentrations of proteins in solutions that were used to spot proteins on the array.

In a related embodiment, provided herein is a method for providing protein array information to a customer, including providing the customer with access over a wide-area network, to identity, quantity and/or quality information regarding a manufactured lot of protein array products, wherein the protein identity, quantity and/or quality information identifies proteins on the array and the concentrations of the proteins on the array. In certain aspects, the array is a microarray or a nanoarray.

Access to the protein array product and/or the protein identity, quantity and/or quality information can be provided on an Internet portal on the wide area network. The quantity information can be concentration of a protein in the solution in which it is spotted on a microarray. In certain aspects, the Internet portal provides automated ordering of a protein array-related product or service based on identification of one or more target proteins on the array. Typically, the target protein is identified by a customer by analyzing proteins on the array for a target activity, a target modification, or a target interaction. For example, target proteins can be proteins on the array identified by a customer as proteins that interact with a test protein used by the customer to probe the array in a protein interaction experiment. Alternatively, for example, target proteins can be proteins on the array identified by the customer as proteins that are substrates for a test enzyme by screening immobilized proteins on the array for the ability of the test enzyme to modify the immobilized proteins.

As indicated above, access to the protein array product and/or the protein identity, quantity and/or quality information can be provided by an Internet portal. The Internet portal can be used by the customer for a variety of additional functions. The Internet portal can be used to identify target proteins and to order related products and/or services. Furthermore, the Internet portal can provide image analysis and data analysis functions. The identity of target proteins based on protein microarray experiments can be entered through the Internet portal to a computer system in a variety of manners. For example, the customer can identify target proteins by selecting one or more target spots on a graphical representation of the protein array displayed on the computer system. The graphical representation of the protein array can be provided on the Internet portal.

The Internet portal can provide access to various analytical functions, including data analysis and image analysis functions, by providing access to one or more programs that perform data analysis and/or image analysis functions. For example, the functions can include a function for identifying positive positions on an image that emit a signal above background (i.e. an image analysis function), a function for identifying relative levels of signals for positive positions using image analysis data and protein identity, quantity and/or quality information (i.e. data analysis function), and/or a function for comparing results of different protein microarray experiments. Another function can compare nucleotide or amino acid sequences to identify identical or similar regions on target proteins. The function can also include one or more functions available in Vector NTI Advance™ 9.0, Vector PathBlazer 2.0, or Vector Xpression™ 3.0 (all of which are available from Invitrogen Corporation, Carlsbad, Calif.), for example as they relate to clones that encode identified target proteins. The functions can be programs that are executed from a remote server using a link on the Internet portal, or the Internet portal can provide a link to a downloadable file that includes one or more of the programs for performing the function that a customer can download to a local computer. In certain illustrative aspects, the functions provided by the Internet portal include those provided by the Prospector microarray analysis program, incorporated herein in its entirety, available on the worldwide web at Invitrogen.com.

Typically, as illustrated in the Examples herein, to perform the image analysis function, after an array is analyzed in an experiment, a desired area of the array is scanned using a scanner after setting or confirming various parameters. Parameters that are set or confirmed can include, for example, wavelength (e.g. 635 nm), PMT gain (e.g. 600), laser power (e.g., 100), pixels size (e.g. 10 um), lines to average (e.g. 1.0), or focus position (e.g. 0 um). Upon scanning of the array, an image of the array is generated and the density of spots on the array is calculated. One or more target proteins are identified by identifying positive positions (i.e. spots) on the array, which are positions that have a higher density of signal than background levels. This can be determined, for example, by identifying those protein spots on the array that have spot densities greater than 3 standard deviations above replicate spots of a control protein. Positive spots on the array can be the result of a protein-protein interaction with a labeled probe, or can be the result of enzymatic modification, such as phosphorylation.

In one aspect, the present invention provides a method wherein a customer sends a microarray that has been probed and optionally dried, to a provider who performs the image analysis function for the customer, for example using the automated tools disclosed herein. An advantage of this is that a customer that does not perform large numbers of protein array experiments does not need to invest in microarray scanning equipment. In one aspect, the service is provided without an additional fee by the provider of the microarray. This aspect provides a provider with an additional opportunity to convince customers to purchase microarrays from the provider.

In another example, a function for comparing microarray results can compare microarray results from a first enzyme assay, such as a kinase assay performed using a microarray and a first enzyme, with results of a microarray that was treated with the first enzyme and then treated with a second enzyme that removes the modification of the first enzyme.

The Internet portal can provide access to various functions that provide images of microarrays. For example, in certain aspects, the Internet portal can provide access to images of arrays wherein a plurality of spots, or every spot, or at least 75%, 80%, 85%, 90%, 95%, 98%, or 99% of the spots are labeled. For example, if proteins spotted on the array are fusion proteins that include a tag such as GST, the image can be obtained based on fluorescence emitted by a fluorescently labeled anti-GST after contacting the microarray. The image of the microarray can be visualized by a customer and compared to results after probing the microarray with a test polypeptide to further evaluate experimental results. Furthermore, the Internet portal can provide images of negative control slides.

In another aspect, the Internet portal provides the ability to compare results from more than one microarray and to normalize data based on signals from control proteins (e.g., GST, BSA, or biotinylated controls). For example, a user can select a microarray compare function that identifies those proteins that are at positive positions in only one of the microarrays being compared, or for which a relative positive signal is different between two microarrays being compared, by more than a cutoff percentage, such as 10%, 15%, 20%, 25%, 50%, 75%, 90%, 95%, or 99%. The relative signals of the protein on two microarrays can be compared by calculating a ratio of the signal at each position of a microarray relative to the signal generated by a control protein, such as a protein known to be expressed at similar levels in various cell types and/or differentiation states and known to bind to a test protein used to probe the array.

In a particular method of the invention, the Internet portal provides access to a protein database, itself forming a separate embodiment of the invention, comprising information related to the identities of protein interactions, substrate information, biological process, and/or biological pathway information. At least some of the information in the database can be identified using a microarray product. The Internet portal can provide further functions that present information in the database in graphical form that can include highlighted regions that include a newly identified target protein and newly identified interactions or enzyme modifications thereof.

In certain embodiments, the methods of the invention further include the steps of designating the customer as a subscriber and enabling the subscriber to access a subscriber-only area of the Internet portal. In certain embodiments, the customer is given permission to add information to the protein database based on information obtained using the microarray.

In other embodiments, the Internet portal provides a data analysis function effective for determining a relative strength of interactions or enzymatic modification between an on-test protein and a protein spotted on the protein microarray product using signal intensity data generated by an image analysis function and protein concentration data from the lot-specific protein concentration information, or the Internet portal provides access to a graphical representation of protein interactions involving polypeptides on the protein array product, or the Internet portal provides access to a graphical representation of protein interactions involving at least 100 polypeptides on the protein array product. In certain embodiments, the Internet portal provides a function for modifying the graphical representation based on the input of protein interaction information identified using the protein array product.

Predictive Methods

In another embodiment, provided herein is a method for predicting an expression pattern, a biological pathway, a biological process and/or enzymatic activity of a test protein, comprising:

a) contacting the test protein with a population of proteins immobilized on a protein microarray;

b) identifying target proteins that bind to the test protein or are that are modified by the test protein; and

c) identifying a common expression pattern, biological pathway, biological process, and/or substrate class among at least some of the target polypeptides, wherein a common expression pattern predicts the expression pattern of the test protein, a common substrate class predicts the type of enzymatic activity of the test protein, and/or a common biological pathway or biological process predicts a biological pathway or biological process involving the target protein. In certain aspects, the test protein is identified and information regarding an expression pattern, biological pathway, biological process, and/or substrate class of the test protein is used to predict the expression pattern, biological pathway, biological process, and/or substrate class. Like other genomic and proteomic technologies, protein micoarray experiments can generate significant amounts of data in relatively short periods of time. This introduces the challenge of representing large amounts of data in an intuitive and understandable form. A further challenge results from the need to integrate this information within the context of known pathways (for example, see S. cerevisiae Genome Database (http://pathway.yeastgenome.org:8555/YEAST/class-subs-instances?object=Pathways), known interactions (for example, see Human Protein Reference Database (http://www.hprd.org) and the literature. This embodiment of the invention, facilitates the process of identifying and analyzing microarray data within the context of biological pathways, biological processes, enzymatic activities, and expression patterns.

For this embodiment, the method can further include presenting a product or a series of products related to the predicted expression pattern, relevant biological pathway and/or type of enzymatic activity for the target protein. For example, a customer can input into the Internet portal, a barcode number and an image of a protein array after probing with the protein array with the test protein and detecting spots on the array to which the test protein binds or modifies. The Internet portal can then activate a function to identify positive positions on the array, identify proteins associated with those positions (i.e. target proteins), and identify common expression patterns, biological pathways, biological processes, and/or substrate classes for the proteins associated with the positive positions on the microarray. Next, the Internet portal can activate a function that uses the identified common expression patterns, biological pathways, biological processes, and/or substrate classes, to query a table of products that are related to specific expression patterns, biological pathways, biological processes, and/or substrate classes.

In a related embodiment, provided herein is a method for predicting an expression pattern, a biological pathway, a biological process, or an enzymatic activity involving a test protein, that includes contacting a plurality of proteins on a protein microarray with the test protein; identifying target proteins on the protein microarray that interact with, or that are modified by, the test protein; and predicting a expression pattern, biological pathway, or biological process involving the test protein by analyzing expression patterns, biological pathways, or biological processes that involve the target proteins. The identification is made by analyzing expression patterns, biological pathways, and/or biological processes that involve one or more of the proteins that interact with, or that are modified by, the test protein. For example, the identification can be performed using a computer program that is stored in a table in computer readable form, which analyzes information regarding expression patterns, biological pathways, and/or biological processes for a plurality of proteins on the protein microarray. For example, the information can be stored in a table of a relational database.

The expression pattern information can include, for example, expression patterns for developmental stages and/or for various tissues within an organism such as within a mammal. The biological pathway information can include a list of all biological pathways, including biochemical pathways, in which a protein is known to be involved. For example, the pathways identified can include any of the pathways in the KEGG database available on the Internet at www.genome.ad.jp. Regarding biological processes, the information can relate to any known biological process, for example, apoptosis, cell division, differentiation, transformation, etc.

For example, it can be determined by probing a protein microarray with a test protein that the test protein interacts with a single target protein on a microarray. By querying a table of information regarding the target protein it is identified that the target protein is involved in apoptosis. Therefore, the method can identify apoptosis as a target biological process for the test protein.

In another hypothetical example, it can be determined by probing a protein microarray with a test protein that the test protein interacts with a series of target proteins on a microarray, all of which are preferentially expressed in the adult liver. Based on this information the method identifies liver expression as a likely expression pattern for the test protein. Accordingly, in one aspect the method further includes the step of comparing expression patterns, biological pathways, biological processes, and/or substrate classes that involve target proteins to identify an expression pattern, biological pathway, biological process, and/or enzymatic activity involving the test protein.

In a related aspect, analysis, identification, and/or prediction steps of the method are performed using computer programs that are accessible by a link on an Internet portal provided herein. In fact, in one illustrative example, a customer can contact a protein microarray with a test protein and a labeled probe that binds the test protein, and upload to the Internet portal, an image of the protein microarray upon detection of the label. Furthermore, information regarding the production lot of the protein microarray can be entered into the Internet portal as well as information regarding the identity and optionally enzymatic activities of the test protein (i.e. probe). An image analysis function and optionally a data analysis function analyze the protein microarray image and identify target proteins that interact or are modified by the test protein based on the image of the protein microarray. The data analysis function can then query a table of information regarding the identified target proteins to identify an expression pattern, biological pathway, or biological process that involves the test protein, as discussed above. The Internet portal can then display the results. For example, a pathway diagram generator function, or pathway assembly function, connected to the Internet portal can generate a diagram of a pathway predicted as involving the test protein. Furthermore, the portion of the pathway that is predicted to involve the test protein can be included or even highlighted. The functions in this embodiment of the invention can be launched automatically, for example, when a user clicks on a hyperlink on an Internet portal. For example, the hyperlink can read “predict biological pathways involving the test protein.”

Accordingly, in this aspect, certain steps of the method are performed by one or more automated functions available from an Internet portal of a provider of protein microarray products where positive interactions or enzymatic modifications are identified from a microarray experiment, and then a customer clicks on one or a series of hyperlinks to visualize the results in the context of various biochemical pathways, expression patterns, or biological processes. The customer does not have to go through the long process of manually entering experimental results. Rather, because the provider knows the identity and location of proteins on the microarrays, for example based on a product number and a lot identifier, the Internet portal can analyze an image of a microarray experiment and display results of predicted expression patterns, biological pathways, and/or biological processes. FIG. 1 provides an example of a pathway diagram that was drawn based on microarray experimental data. The Internet portal can be configured such that a user can enter data such as a lot number for the microarray, a scanned image of a microarray experiment, identify the test protein (i.e. probe for the microarray), and optionally identify the type of experiment, e.g. protein interaction or enzymatic activity, for example a specific type of enzymatic activity assay, and within 10, 9, 8, 7, 6, 5, 4, 3, 2, or as an illustrative example 1 user action, such as clicking on a hyperlink, one or more pathway maps are presented to the customer. For example, the pathway maps can be displayed on the Internet portal, e-mailed to the user, and/or presented to the user for download.

In certain aspects of the invention, the method for predicting an expression pattern, a biological pathway, an enzymatic activity, or a biological process involving a test protein, further includes performing another method such as a protein expression profiling technique, for example using an antibody array (Schweitzer, B. and S. F. Kingsmore, Measuring proteins on microarrays. Curr Opin Biotechnol, 2002. 13(1): p. 14-9), tissue array (Ziauddin, J. and D. M. Sabatini, Microarrays of cells expressing defined cDNAs. Nature, 2001. 411(6833): p. 107-10), gel electrophoresis, such as 2-dimensional gel electrophoresis, mass spectrometry (Aebersold, R. and D. R. Goodlett, Mass spectrometry in proteomics. Chem Rev, 2001. 101(2): p. 269-95), and/or an mRNA profiling technique (Schena, M., et al., Quantitative monitoring of gene expression patterns with a complementary DNA microarray. Science, 1995. 270(5235): p. 467-70) to validate predictions made using the methods for predicting, disclosed herein.

In certain aspects provided herein, datasets from a microarray analysis experiment and at least one of the other protein and expression analysis methods listed above, are presented in forms which are sufficiently structured such that cross-referencing between experiments is straightforward. For example, XML standards can be utilized. Furthermore, provided herein is a software tool that integrates data-types into forms which can be easily interpreted by users.

For example, in certain aspects micrarray results are presented using one of the functions of Vector PathBlazer 2.0™. For example, results can be displayed in the context of a pathway and can be comined with public data. The functions can be available using software running on a local computer or a computer connected over a wide-area network, such as an Internet server that is accessed through an Internet portal, such as the Internet portal disclosed herein.

Furthermore, in certain aspects, targeted proteins identified using protein microarrays provided herein are listed as accession numbers that include hyperlinks that customers can select to go to information regarding the target protein, such as information about the sequence of the target protein, literature and patent references disclosing the target protein, the structure of the target protein, and/or biological pathways that include the target protein.

Links to Products and Services

In addition to various data analysis functions, the Internet portal can present to the customer, links to one or more products and/or services (i.e. a product or service or a plurality of products and/or services) related to protein arrays (i.e. related products and/or services). The links to related products and services can be customized based on the target proteins identified experimentally using a protein microarray.

The target protein can be identified by a customer by analyzing proteins on the array for a target activity or a target interaction. For example, target proteins can be proteins on the array identified by a customer as proteins that interact with a first protein, sometimes referred to herein as the test protein or the probe, used by the customer to probe the array in a protein interaction experiment. Alternatively, for example, target proteins can be proteins on the array identified by the customer as proteins that are substrates for a first enzyme used by the customer to screen immobilized proteins on the array for their ability to be modified by the first enzyme.

In another aspect, target proteins can be identified by functions provided by the Internet portal based on scanned images of protein array experimental results. For example, am image analysis function after identifying target positions (i.e. target spots), identifies target proteins associated with the positive positions using manufactured lot-specific identity information, and automatically transmits target protein identities to the Internet portal. A function then links identified target protein to related products and/or services. For example, the function can include a table and/or database that for every target protein, identifies related products and services.

Related products and services include any product and service whose use or function is associated with further production, analysis and/or characterization of an identified target proteins, or validation of microarray results. In certain aspects the products or services include the same type of product or service for all the target proteins. For example, the product and service can be any product or service available on the Internet at Invitrogen.com (incorporated herein by reference in its entirety). Exemplary products offered by the provider can include clone collections and individual clones, polypeptides, such as enzymes, antibodies, libraries (e.g., cDNA libraries, genomic libraries, etc.), buffers, growth media, purification systems, primers, cell lines, chemical compounds, fluorescent labels, functional assays, and a variety of kits including protein interaction identification kits, kits for performing enzymatic assays, and kits for performing DNA and protein purification, amplification and modification. Further, these exemplary products are provided for example only and are not intended to limit the present invention. Exemplary services offered by the provider include clone construction services, protein expression services, antibody production services, library (e.g., cDNA library, genomic library, etc.) construction services, and research and development consulting services. As a more specific example, the product or service can be one or a series of antibodies or the generation thereof, isolated proteins or the production thereof, clones that encode and optionally express target proteins or the production thereof, primers for amplifying nucleic acids encoding one or more of the target proteins, protein separation reagents, protein-protein interaction reagents or the production of products used in a protein-protein interaction experiment, an enzymatic assay or the production thereof, solid supports or other chromatography reagents that include one or more target proteins, protein molecular weight markers, RNA or cDNA from various developmental stages or the production thereof, homologs of target proteins in other species, RNAi for down-regulating expression of a target protein, or clones that encode such homologs, or the production thereof, enzyme substrates, and/or additional microarrays, for example microarrays coated on a different substrate that than the substrate used to identify the target proteins.

In one aspect, the related product or service presented to a user depends on an enzymatic or other biological activity of the test protein (i.e. probe). For example, the product or service presented can relate to identification of a protein immobilized on a protein array as a substrate for a kinase. The product or service can validate that the immobilized protein is a substrate for a kinase, for example.

In fact, in a separate embodiment of the methods herein, is provided a method for identifying a substrate for a kinase that includes contacting the kinase with a series of proteins on a microarray and a labeled source of phosphate, such as labeled ATP. Then the label is detected on proteins of the microarray contacted with the kinase (i.e. kinase-treated microarray), and the kinase-treated microarray is contacted with a phosphatase that is specific for the type of kinase activity of the kinase. Then labeled proteins on the microarray contacted with the phosphates are detected and the phosphatase treated microarray results are compared with those of the kinase-treated microarray. Proteins that were phosphorylated by the kinase and dephosphorylated by the phosphatase are identified as substrates for the kinase. For example, if the kinase is a tyrosine kinase, the phosphatase can be a phosphotyrosine phosphatase. If the kinase is a serine/threonine kinase, the phosphatase can be a phosphoserine/phosphothreonine phosphatase.

In one aspect, the product or service is a plurality of products or services that are identical products or services except that they pertain to a different protein of the target proteins. For example, if 1, 2, 3, 4, 5, 10, 15, 20, 25, 50, or 100 proteins are identified which interact with a test protein, a customer could order 1, 2, 3, 4, 5, 10, 15, 20, 25, 50, or 100 antibodies, each of which binds a different protein of the 1, 2, 3, 4, 5, 10, 15, 20, 25, 50, or 100 target proteins, 1, 2, 3, 4, 5, 10, 15, 20, 25, 50, or 100 clones, each of which expresses on of the 1, 2, 3, 4, 5, 10, 15, 20, 25, 50, or 100 target proteins.

In one example, the products and/or services can relate to a validation method that validates results generated using a protein array. In aspects wherein a protein-protein interaction experiment is performed using a protein array, the products and/or services can be products used in a protein interaction validation method. For example, the products and/or services can be a plurality of products and/or services that are used in a yeast two hybrid experiment (Uetz et al., 2000, Nature 403:623; Ito et al., 2000, Proc. Natl. Acad. Sci. U.S.A.97: 1143), a co-immunoprecipitation reaction, a gel shift assay, or for determining reciprocal interactions using a second protein array. Additional methods for validating protein-protein interactions include the Verve Mammalian two-hybrid kit, the ProQuest two-hybrid system with Gateway technology, the Hybrid Hunter Yeast Two Hybrid System and the Dual Bait Hybrid Hunter Yeast Two-Hybrid System (Invitrogen, Carlsbad, Calif.). In another aspect where one or more substrates for a kinase are identified using the protein arrays provided herewith, links to one or more related reagents for a solution-based kinase assay are provided to the customer so that the customer can validate the results of the substrate identification initially performed using a protein microarray. In certain illustrative aspects, the related reagents are kits for identifying substrates of the kinase using a solution-based assay, such as the solution kinase assay kits available from Invitrogen Corporation (Carlsbad, Calif.).

In certain aspects of the invention, a customer is presented a plurality of products or services wherein the plurality is customized based on positive spots identified by the customer. The products can include, for example, clones, nucleic acids, isolated proteins, as well as kits for performing confirmatory reactions. Services can include, for example, protein production and isolation as well as antibody production.

In another embodiment, the product or service, or the plurality of products or services, comprises a protein array product comprising a plurality of arrayed spots comprising the target proteins. In another embodiment, each array of spots comprising the target proteins of the plurality of arrayed spots is located within a well of a plurality of wells on the array. In another embodiment, the product or service, or the plurality of products or services, comprises a plurality of primers against nucleic acids encoding the target proteins, or amplifying the nucleic acids encoding the target proteins.

In an embodiment of the invention, the method further comprises providing the customer with access to one or more clone products corresponding to one or more proteins on the protein array product. In another embodiment, the clone products comprise nucleic acid sequences in a recombinational vector.

In certain embodiments, the products or services related to protein arrays include one or more isolated proteins corresponding to one or more target proteins on the protein array product identified by the customer. In certain embodiments, the protein array product comprises at least 1000 proteins, or at least 100 proteins. In other embodiments, the proteins are from the same species and the proteins are from, e.g., yeast, human, mouse, rat, dog, or monkey. The proteins may be from a pathogen, or include homologs from more than one species, or share a common biological activity.

In other embodiments, the products or services provided to a customer are customized based on the protein array product selected by the customer. In certain methods of the invention, directed access to the customer to a product or service, or to a plurality of products or services is provided in between one and five computer user actions after identification of target proteins, or is provided within one computer user action . For example, after target proteins are identified on the Internet portal, antibodies against the target protein(s) can be presented for purchase to the customer on a computer display, and the customer can purchase the antibodies within one, two, three, four, five, six, seven, eight, nine, or ten, preferably five or less, more preferably three or less computer user actions. Computer user actions include, for example, clicks of a mouse or similar device, spoken commands, touch screen touches, or other actions that a user performs to provide an input signal to a computer. A user computer action is typically performed by selecting a target choice in response to a series of choices that are presented to a user on a display, such as a computer monitor. In one aspect, the user computer action is the selection of a hyperlink that is presented to the user. For example, the hyperlink can be one of a series of hyperlinks presented to a user on the Internet portal. In one embodiment, the Internet portal displays a link that can be clicked on by a customer to order a plurality of antibodies, each of which recognizes a different target protein. Therefore, if 25 target proteins are identified, by clicking on a link on the Internet portal, antibodies against the 25 proteins can be ordered by a customer. The 25 antibodies are then shipped to the customer without further customer intervention, or after the customer enters payment information, such as an account number or a credit card number. The antibodies, if they do not already exist in inventory by the provider, can be generated.

In one embodiment, the product or service or plurality of products and services presented to the customer, comprises a protein separation product. In certain methods of the invention, the molecular weight range of proteins effectively separated by the separation product presented to the customer is identified based on the molecular weight of proteins in protein spots at identified positive signals.

In other methods, the product or service or plurality of products or services, includes presenting to the customer, access to purchasing at least one clone of a clone collection database, wherein the at least one clone is identified based on an identified positive signal. In a particular embodiment, the clone collection database is divided into a private area and a public area. Furthermore, the clone collection database contains information identifying the characteristics of individual members of a clone collection. In other embodiments, the customer is presented with access to an expression database containing information identifying optimized expression sequences for expressing a nucleic acid molecule encoding a protein identified by the positive spots.

In certain aspects of the methods provided herein, access to a product or service, or to a plurality of products or services is provided by between one and five computer user commands, for example mouse clicks, after identification of positive positions on a microarray after a microarray experiment. In an illustrative example, access to a product or service, or to a plurality of products or services is provided by one mouse click after identification of positive signals. For example, when positive positions on a microarray are graphically presented, a hyperlink can be presented that is associated with text such as “Click here for related products and/or services.” When the user clicks the hyperlink, a list of products and/or services can be presented to the customer, or a series of options can be presented such as “isolated proteins,” “antibodies,” “nucleic acid probes,” “clones,” or “services” that when selected link to Internet pages with the appropriate product and/or service that can be customized based on the identified target protein(s).

In a related embodiment, provided herein is a method for providing protein array information to a customer, comprising:
a) providing to the customer, an automated system for ordering a protein array from a lot of protein arrays; and
b) providing the customer with access to a database comprising information regarding the identity and quantity of proteins on the protein array.

In another related embodiment, provided herein is a method for determining a relative strength of interaction between a target protein on a protein array and a probe, or a relative level of enzymatic modification of the target protein on the protein array by the probe, comprising
a) performing an assay by contacting proteins on a protein array with the probe to identify a positive signal on the array;
b) obtaining information regarding the identity and quantity of proteins on the protein array from a supplier of the protein array;
c) identifying the protein associated with the positive signal using the information regarding the identity and quantity of proteins on the protein array; and
d) identifying the strength of interaction or enzymatic modification of the target protein on the protein array using the information regarding the identity and quantity of proteins on the protein array.

In another related embodiment, provided herein is a method for determining a strength of interaction between a target protein on a protein array and a probe or a strength of enzymatic modification of the target protein on the protein array by the probe, comprising
a) providing an assay by contacting proteins on a protein array with the probe to identifying a positive signal on the array;
b) obtaining information regarding the identity and quantity of proteins on the protein array from a supplier of the protein array;
c) identifying the protein associated with the positive signal using the information regarding the identity and quantity of proteins on the protein array; and
d) identifying the strength of interaction or enzymatic modification of the target protein on the protein array using the information regarding the identity and quantity of proteins on the protein array.

In certain embodiments of the invention, the method further includes identifying similar expression and/or biological pathway patterns for target polypeptides identified using the protein array product. The identification is typically performed by a computer program to which access is provided in a link on the Internet portal. The identified similar expression and/or biological pathway patterns can be presented to the customer. The customer can be presented with a customized, selectable list of products and services related to the identified expression and/or biological pathway patterns.

In another embodiment, provided herein is a database, comprising information related to the identities of protein interactions, substrate information, and/or biological pathway information, identified using a protein microarray product. The database can include information regarding at least 100 related proteins. The at least 100 proteins can be from the same species of organism and/or the at least 100 proteins have the same biological activity. In another embodiment, provided herein is a graphical representation of the database discussed above, wherein the graphical representation is provided in computer readable form.

In another embodiment, provided herein is a method for predicting a site of enzymatic modification, comprising:

a) identifying polypeptide substrates for an enzyme using a protein microarray comprising a population of polypeptides; and

b) comparing amino acid sequences of the polypeptide substrates to predict a site of enzymatic modification.

The polypeptide substrates can be a substrate for virtually any type of substrate, especially a substrate that is detectably modified by the enzyme. For example, the substrate can be a kinase substrate. The sit of enzymatic modification in certain aspects, for example is a phosphorylation site.

Method for Determining Protein Concentration

As methods involving hundreds, thousands, tens of thousands, or more proteins become common, there is a need for methods that allow efficient quantitation of protein concentrations for large numbers of proteins. Accordingly, in another embodiment, provided herein is a method for determining the concentration of a target protein, including:
a) providing a protein microarray including a spot of the target protein comprising a tag and a series of spots derived from solutions comprising different known concentrations of a control protein comprising the tag;
b) contacting the protein microarray with a first specific binding pair member that binds the tag;
c) determining a level of binding of the first specific binding pair member to the tag on the target polypeptide and to the different known concentrations of the control protein comprising the tag; and
d) determining the concentration of the target protein using the level of binding of the first specific binding pair member to the tag on the target polypeptide and the level of binding of the first specific binding pair member to the different known concentrations of the control protein comprising the tag. The concentration is usually determined using a cubic curve fitting method

The number of tags on the control protein and the target protein are typically known. For example the control protein and the target protein can include one tag molecule per protein molecule. Therefore, the method typically involves immobilizing a series of tagged control proteins of different known concentrations at a series of locations on a microarray to provide a series of spots of the tagged control proteins. Signals obtained for the series of tagged control protein spots after probing, for example with a fluorescently labeled antibody against the tag are used to generate a standard curve that is used to determine a concentration of one or more target polypeptides. The target polypeptides are typically spotted on the same array. In an illustrative embodiment, the tag is glutathione S-transferase.

For example, the tagged control protein on the series of spots can be present in a concentration of between about 0.001 ng/ul and about 10 ug/ul, between 0.01 ng/ul and 1 ug/ul, between 0.025 ng/ul and 100 ng/ul, between 0.050 ng/ul and 75 ng/ul, between 0.075 ng/ul and 50 ng/ul, or, for example, between 0.1 ng/ul and 25 ng/ul. In one specific embodiment, the tagged control protein can be present at a series of spots at a concentration of tagged control protein of between 0.1 ng/ul and 12.8 ng/ul.

Each target protein and control protein is usually spotted in more than one spot to provide further statistical confidence in values obtained. In certain example, concentration is determined for a plurality of target proteins, for example at least 100, 200, 250, 500, 750, 1000, 2000, 2500, 5000, 10,000, 20,000, 25, 000, 50,000 or 100,1000 target proteins. Each of the target proteins is typically immobilized on the same microarray, but can also be immobilized on a series of microarrays. For example, concentration can be determined for between 10 an 1,000,000 proteins, between 50 and 100,000 proteins, between 100 and 20,000 or between 250 and 10,000. Therefore, an advantage of the present invention is that large numbers of proteins can be analyzed in the same experiment using the same standard curve.

In methods provided herein, the concentration is typically determined using a cubic curve fitting method having the following formula:
Y=a*X³+b*X²+c*X

Where X is the spot relative intensity and the Y is the spot protein concentration. The fitting formula is used to calculate all other proteome spots in the slides. Open source software Polyfit is applied for this curve fitting purpose. In order to get a designed polynomial like Y=a*X³+b*X²+c*X+d with d=0, instead of using Polyfit the usual way, we create a new function Y′=Y/X=a*X²+b*X+c, using Polyfit for 2nd order, we get coefficients a, b, c, then use this a, c, b for the 3-rd order polynomial

Because the protein concentration of the control spots is known and the intensity can be obtained from the uploaded result file, a fitting curve can be created and the correspondent fitting formula based on the control spots' intensity and concentration. The cubic curve fitting method is applied.

The tag on the tagged control can be an affinity purification tag as discussed in further detail herein. The affinity purification tag can be, for example, glutathione S-transferase. In certain aspects, the present invention provides a microarray comprising a plurality of concentration series of tagged control proteins, wherein at least two of the series comprise different tags. Accordingly, another aspect of the invention is a microarray with a plurality of concentration series of tagged control proteins. A concentration series is a series of protein spots of different known concentrations used to construct a standard curve and associated formula for determining a concentration of an unknown protein. For example, a microarray can include 1, 2, 3, 4, 5, 6, 7, 8, 9, 10, 15, 20, or 25 separate concentration series, and although each tagged protein of a series typically includes the same tag, tagged control proteins of different series can include different tags. Therefore, a microarray with multiple concentration series can be sold to a customer for use in determining protein concentrations for proteins that are tagged with any tag represented in a series that is attached to a target protein. In other words, a microarray with multiple concentration series with different tags provides a robust tool that can be used to determine concentration of a target protein for many different tags.

In certain embodiments of the present invention, the concentration of a protein on an array refers to the concentration of the protein in solution when the protein was initially deposited on the array. Therefore, although the contacting and detecting are performed when the target protein is immobilized, the concentration of the target protein in solution is determined using the standard curve.

The method for determining the concentration of a target protein can be used to determine the concentration of 10, 15, 20, 25, 50, 75, 100, 200, 250, 500, 750, 1000, 2000, 2500, 5000, 10,000, 20,000, 25,000, 50,000, 100,000, 200,000, 250,000, 500,000, 750,000, 1,000,000 proteins or more target proteins. The target proteins can be spotted onto 1, 2, 3, 4, 5, 6, 7, 8, 9 or 10 microarrays. In an illustrative embodiment, the target proteins are spotted onto 1 microarray to determine the concentrations of the target proteins.

In one aspect of the method provided herein, protein concentrations are determined by using an equivalent solution protein concentration calculation. Each lot of microarray slides is spotted with a known concentration gradient of purified GST protein. Representative arrays are probed with an anti-GST antibody and the resulting signal is used to calculate a standard curve. This standard curve is then used to calculate the equivalent solution protein concentration of the proteins spotted on the arrays. The intensity of signals for the GST protein gradient present in every subarray is used to calculate a standard curve from which the equivalent solution concentrations of all the yeast proteins are extrapolated. This measure is not an absolute amount of protein on the array but reflects the expected solution concentration for each protein. For a protein reported as having an “equivalent solution concentration” of 10 ng/μl, one can use the quantity spotted to determine the quantity of protein on the microarray. For example, 10 pg of protein can be spotted in a single spot.

In a related embodiments, a service is provided for determining the concentration of a plurality of target proteins. For example, the service can receive target proteins from a customer, spot the proteins on a microarray, and perform the method provided above for determining the concentration of a target protein.

Method for Validating Labeling of a Probe

In certain embodiments the invention provides a method for validating labeling of a probe, including contacting the labeled probe with a control polypeptide, wherein the control polypeptide is capable of specific binding to the label; and wherein a positionally addressable control microarray includes the control polypeptide; and detecting binding between the probe and the control polypeptide, wherein binding between the probe and the control polypeptide indicates that the labeling of the probe is validated.

Aspects of the invention that include a control microarray typically involve a multi-step process wherein labeling of the polypeptide is validated or detected before the labeled polypeptide is contacted with a test microarray. This allows a researcher to assure that a biotinylation reaction of a protein of interest (i.e. a test protein) is successful before a test microarray, which is typically a more valuable microarray, is contacted with the labeled test polypeptide.

In another embodiment, provided herein is a method for assessing the level of biotinylation of a probe, which is typically a polypeptide. The method is typically performed using SDS-PAGE of biotinylated probe samples and a biotinylated gel standard. A dilution series is prepared from a biotinylated gel standard that includes a known level of biotinylation. SDS-PAGE is then performed using biotinylated probe samples and the dilution series of the biotinylation gel standard. The separate proteins are then transferred to a nitrocellular member. Immunoblot analysis is then performed using a streptavidin labeled probe to verify and asses the level of biotinylation of the probe.

Any method known to the skilled artisan can be used to label a probe. The probe can be, but is not limited to, a peptide, polypeptide, protein, nucleic acid, or organic molecule. The label can be, but is not limited to, biotin, avidin, a peptide tag, or a small organic molecule. The label can be attached to the probe in vivo or in vitro. Where the label is biotin, the label can be bound to the probe in vitro or vivo using commercially available reagents (Invitrogen, Carlsbad, Calif.). For example, the probe can be a protein probe labeled in vivo with a biotin label, using a fusion protein that includes a peptide to which biotin is covalently attached in vivo. For example, a Bioease™ tag (Invitrogen, Carlsbad, Calif.) can be used. The BioEase™ tag is a 72 amino acid peptide derived from the C-terminus (amino acids 524-595) of the Klebsiella pneumoniae oxal acetate decarboxylase a subunit (Schwarz et al., 1988). Biotin is covalently attached to the oxalacetate decarboxylase α subunit and peptide sequencing has identified a single biotin binding site at lysine 561 of the protein (Schwarz et al., 1988, The Sodium Ion Translocating Oxalacetate Decarboxylase of Klebsiella pneumoniae, J. Biol. Chem. 263, 9640-9645, incorporated herein in its entirety by reference). When fused to a heterologous protein, the BioEase™ tag is both necessary and sufficient to facilitate in vivo biotinylation of the recombinant protein of interest. The entire 72 amino acid domain is required for recognition by the cellular biotinylation enzymes. For more information about the cellular biotinylation enzymes and the mechanism of biotinylation, refer to the review by Chapman-Smith and Cronan, 1999 (,Chapman-Smith, A., and J. E. Cronan, J. (1999). Molecular Biology of Biotin Attachment to Proteins, J. Nutr. 129, 477S-484S. incorporated herein in its entirety). In certain specific embodiments, the label is attached to the probe via a covalent bond. The methods of the invention allow verification of the labeling of the probe. In certain, more specific embodiments, the methods of the invention also allow quantification of the labeling of the probe, i.e., what proportion of the probe in a sample of the probe is labeled.

The control polypeptide is a polypeptide that is capable of binding to the label. In a specific embodiment, the label is biotin and the control polypeptide is an anti-biotin antibody. In certain embodiments, a control molecule can be used; such a control molecule is capable of binding the label, and can be, but is not limited to, a nucleic acid or small organic molecule.

In certain embodiments, the control peptide is immobilized on a microarray, termed a “control microarray.” In certain, more specific embodiments, the control microarray also includes controls that test for nonspecific binding (also referred to herein as unspecific binding) of the probe to the surface of the array and/or nonspecific binding of the probe to protein.

In certain embodiments, the labeled protein is used to screen a positionally addressable protein microarray. In certain, more specific embodiments, at least some of the proteins on the positionally addressable protein microarray comprise a peptide or polypeptide tag (e.g., a peptide or polypeptide tag that was used to purify the protein and/or to immobilize the protein to the microarray). If such peptide tags are present on the positionally addressable protein microarray then the control array may also comprise a control for nonspecific binding of the probe to the peptide or polypeptide tag. Such a control could be a peptide or polypeptide tag gradient, ie, several positions of the control array would have different concentrations of the peptide tag.

The invention further provides a method for probing a test microarray, the method including contacting a first aliquot of a labeled probe with a control polypeptide, wherein the control polypeptide is capable of specific binding to the label; and wherein a positionally addressable control microarray comprises said control polypeptide; detecting binding between the probe and the control polypeptide; and contacting the test microarray with a second aliquot of the labeled probe, wherein the first aliquot and the second aliquot of the labeled probe were prepared in the same reaction, and wherein the test microarray comprises a plurality of proteins; and detecting binding between the labeled probe and a protein of the test microarray.

In an embodiment provided herein that is related to the method for validating lableling of a probe, the labeled probe is referred to as a labeled test polypeptide and the label is referred to as a first specific binding pair member of a first binding pair. Furthermore, in this embodiment, the control polypeptide includes a second specific binding pair member of the first binding pair. According this embodiment provides a method for detecting labeling of a test polypeptide, including labeling the polypeptide with a first specific binding pair member of a first binding pair; contacting the labeled polypeptide with a control polypeptide on a control microarray, wherein the control polypeptide comprises a second specific binding pair member of the first binding pair; and contacting the test microarray with the labeled polypeptide, wherein detectable binding of the labeled polypeptide to the second specific binding pair member of the first binding pair is indicative of labeling of the polypeptide, thereby detecting labeling of the test polypeptide. In an illustrative example, the second specific binding pair member is an antibody that specifically binds the label and the label is biotin.

The method for validating labeling of a probe or method for detecting labeling of a test polypeptide, can further include analyzing the labeling of the test polypeptide or probe by a traditional method. In these embodiments, the method includes both a step where the labeled polyeptide is contacted with a control polypeptide on a control microarray and a step of analyzing the labeling of the polypeptide by a traditional method. For example, the traditional method can include gel electrophoresis and immunoblotting, as disclosed in the Examples herein. In certain aspects, the traditional method or the control microarray method can assess the quantity and/or quality of labeling. In certain aspects, the invention can further include quantitating the labeling of the probe using mass spectroscopy.

In certain aspects, removal of the label not covalently attached to the labeled test polyeptide is analyzed. For example, binding of the labeled polypeptide to the control microarray in regions outside of the spot containing the control polypeptide can be detected. Excessive signal from binding to regions outside of the spot containing the control polypeptide indicates that excess free label remains. Typically, an identical substrate is used for the test microarray and the control microarray.

In certain aspects of the invention, the control array further includes a first specific binding pair member (SBP) of a second binding pair, and the method further includes contacting the control array with a second specific binding pair member of the second binding pair. Furthermore, the method can include labeling the second specific binding pair member of the second specific binding pair with the first specific binding pair member of the first binding pair before contacting the control array with the second specific binding pair member of the second binding pair. For example, as illustrated herein the first SBP of the second binding pair can be a polypeptide such as calmodulin kinase, that binds to a protein on the control microarray, such as calmodulin. The first SBP of the second binding pair is referred to in the illustrative example as a control interacting protein. The first SBP of the second binding pair, e.g., calmodulin, can be labeled for example with biotin, in examples where the test polypeptide is also labeled with biotin. This labeling can be performed using the same biotinylation solutions as are used to label the test polypeptide. In this example biotinylated calmodulin and biotinylated test protein indirectly bound to the microarray through binding of a binding pair member, can be contacted with fluorescently labeled streptavidin to detect the indirect binding to the microarray. Binding of calmodulin to the calmodulin kinase indicates that a biotinylation reaction was successful. Binding of the biotinylated test polypeptide to the antibody against biotin indicates that the biotinylation reaction of the test polypeptide was successful. Typically, biotinylation of the test polypeptide results in more than 1 biotin moiety per test polypeptide. Therefore, an antibody against biotin as well as labeled streptavidin can bind to the test polypeptide at the same time.

Other control elements can be present on the control microarray or as part of the test microarray. For example, BSA spots and buffer spots can be included on the control microarray and the test microarray to detect nonspecific binding. Furthermore, fluorescent molecules can be directly bound to the microarray as markers to assess orientation of the microarray.

Computer Program Products and Methods

In certain embodiments, the invention provides a computer program product for use in conjunction with a computer system, the computer program product comprising a computer readable storage medium and a computer program mechanism embedded therein, the computer program mechanism comprising one or more data structures, said one or more data structures dimensioned and configured to store information comprising location, identity, and concentration of proteins present on a protein array, for a plurality of protein arrays; instructions for inputting an identifier associated with a purchased or obtained protein array present in said plurality of protein arrays; instructions for outputting said information regarding the protein array corresponding to said identifier. In certain embodiments, the instructions are computer-executable instructions in a computer language.

In certain embodiments, the protein arrays are manufactured in lots such that the information associated with each protein array in one lot is the same. In certain embodiments, the information for a plurality of lots of protein arrays is stored in the data structure.

In certain embodiments, the invention provides a method implemented by a computer system coupled to a wide-area network, the method comprising user inputting of an identifier associated with a purchased or obtained protein array; and retrieving, over the wide-area network, characteristics associated with the protein array associated with said identifier.

In certain embodiments, the invention provides a computer program product for use in conjunction with a computer system, the computer program product comprising a computer readable storage medium and a computer program mechanism embedded therein, the computer program mechanism comprising: instructions for user inputting of an identifier associated with a purchased or obtained protein array; and instructions for outputting characteristics associated with the protein array associated with said identifier.

In certain, specific embodiments, the wide-area network is the internet. In certain, specific embodiments, the information can be stored on and retrieved from a disk.

The invention further provides a computer comprising a central processing unit; and a memory, coupled to the central processing unit, the memory storing: a) one or more data structures, said one or more data structures dimensioned and configured to store information comprising location, identity, and concentration of proteins present on a protein array, for a plurality of protein arrays; b) instructions for inputting an identifier associated with a purchased or obtained protein array present in said plurality of protein arrays; c) instructions for outputting said information regarding the protein array corresponding to said identifier.

In another embodiment, a computer product of the invention provides an Internet portal or a function provided thereon, for data analysis of protein microarray results, as discussed herein. The Internet portal provides access to various functions, including data analysis and image analysis functions, by providing access to one or more programs that perform data analysis and/or image analysis functions. The programs can be executed from a common server using a link on the Internet portal or the Internet portal can provide a link to a downloadable file that includes one or more of the programs, that a customer can download to a local computer.

Protein Arrays

The methods, kits, and systems provided herein include a positionally addressable array that includes a plurality of proteins, with each protein being at a different position on a solid support. The present invention also encompasses a positionally addressable array including a plurality of proteins, with each protein being at a different position on a solid support, wherein the plurality of proteins includes at least 1, 2, 3, 4, 5, 10, 20, 30, 40, 50, 100, 200, 500, 1000, 1500, 2000, 2500, 3000, 4000, 5000, 6000, 7000, 8000, 9000, 10,000, 20,000, 25,000, 50,000, 75,000, 100,000, 500,000 or 1,000,000 different proteins. The proteins on the test array, in certain illustrative aspects, are related proteins. Related proteins are typically proteins of the same protein family, enzyme class, biological pathway, species, or related group of species, such as the same genera.

In one aspect, the protein array is a bead-based array. In another aspect, the protein array is a planar array.

The present invention also encompasses a positionally addressable array including a plurality of proteins, with each protein being at a different position on a solid support, wherein the plurality of proteins in aggregate comprises at least 1, 2, 3, 4, 5, 10, 20, 30, 40, 50, 100, 200, 500, 1000, 5000, 10000, 20000, 30000, 40000, or 50000 different known genes in a single species. In one aspect, the plurality of proteins includes at least one protein encoded by at least 1%, 2%, 3%, 4%, 5%, 10%, 20%, 30%, 40%, 50%, 60%, 70%, 80%, 90%, 95%, or 99% of the known genes in a single species, i.e., all protein isoforms and splice variants derived from a gene are considered one protein. In one aspect, the plurality of proteins comprises about 90%, 95%, or 99% of all proteins expressed in a species. In a particular embodiment, the plurality of proteins comprises about 93.5% of all proteins expressed in a species.

The present invention also encompasses a positionally addressable array comprising a plurality of fusion proteins to a surface of a solid support, with each fusion protein being at a different position on the solid support, wherein the fusion protein comprises a first tag, a second tag, and a protein sequence encoded by genomic nucleic acid of an organism. In another embodiment, the protein sequence of the fusion protein need not be encoded in a genomic nucleic acid of an organism, but is a sequence for which it is desired to identify a function and/or activity of a binding protein.

A positionally addressable array provides a configuration such that each probe or protein of interest is at a known position on the solid support thereby allowing the identity of each probe or protein to be determined from its position on the array. Accordingly, each protein on an array is preferably located at a known, predetermined position on the solid support such that the identity of each protein can be determined from its position on the solid support.

In certain methods of the present invention, the protein array product is a protein microarray comprising at least one hundred enzymes of the same class of enzymes, or the protein microarray comprises a substrate for at least one enzyme of the class of enzymes. In a particular method, the protein array product is other than an antibody array. In certain illustrative examples, the protein microarray does not include more than 1, 2, 3, 4, 5, 6, 7, 8, 9, 10, 15, or 20 antibodies. The protein array product can include a protein microarray with at least one hundred enzymes of the same class of enzymes. In one aspect, the protein microarray product can further include an enzyme substrate for at least one enzyme of the class of enzymes. The enzyme substrate can be immobilized on the microarray substrate or it can be provided in a separate container.

In one aspect, the proteins on the microarray are from a virus species. In another embodiment, the species is a prokaryote. In another embodiment, the species is a eukaryote. In another embodiment, the species is a vertebrate. In yet another embodiment, the species is a mammal. In a particular embodiment, the species is an animal, including, but not limited to, an insect, primate, and rodent. In a specific embodiment, the species is a monkey, fruit fly, cow, horse, sheep, pig, chicken, turkey, quail, cat, dog, mouse, rat, rabbit, nematode or fish. In a preferred embodiment, the species is a human. In another preferred embodiment, the species is a yeast. The proteins on microarrays provided herein can include control proteins that are not related to the majority of proteins on a microarry, which are related.

Proteins of the protein microarrays used in methods, kits and systems of the invention include full-length proteins, portions of full-length proteins, and peptides, which can be prepared by recombinant overexpression, fragmentation of larger proteins, or chemical synthesis. Proteins can be overexpressed in cells derived from, for example, yeast, bacteria, insects, humans, or non-human mammals such as mice, rats, cats, dogs, pigs, cows and horses. Further, fusion proteins comprising a defined domain attached to a natural or synthetic protein can be used. Proteins of the protein microarrays can be purified prior to being attached to the solid support of the chip. Also the proteins of the protein microarrays can be purified, or further purified, during attachment to the protein microarray.

Proteins can be embedded in artificial or natural membranes (e.g., liposomes, membrane vesicles) prior to, or at the time of attachment to the protein chip. In fact, the synthesis of certain proteins may preferably be conducted in the presence of artificial or natural membranes to, for example, promote protein folding, protein processing, retain activity, and/or prevent precipitation of the protein.

Further, proteins can be attached to the solid support of the protein microarray. Alternatively, the proteins can be delivered into wells of the protein microarray, where they remain unbound to the solid support of the protein microarray.

A variety of solid supports can be used for protein microarrays for the methods, kits, and systems provided herein. The solid support can be constructed from materials such as, but not limited to, silicon, glass, quartz, polyimide, acrylic, polymethylmethacrylate (LUCITE®), ceramic, nitrocellulose, amorphous silicon carbide, polystyrene, and/or any other material suitable for microfabrication, microlithography, or casting. For example, the solid support can be a hydrophilic microtiter plate (e.g., MILLIPORE™) or a nitrocellulose-coated glass slide. In a preferred embodiment, the solid support is a nitrocellulose-coated glass slide. Nitrocellulose-coated glass slides for making protein (and DNA) microarrays are commercially available (e.g., from Schleicher & Schuell (Keene, N.H.), which sells glass slides coated with a nitrocellulose based polymer (Cat. no. 10 484 182)). In a specific embodiment, each protein is spotted onto the nitrocellulose-coated glass slide using an OMNIGRID™ (GeneMachines, San Carlos, Calif.). The present invention contemplates other solid supports useful for constructing a protein chip, some of which are disclosed, for example, in co-pending U.S. application Ser. No. 09/849,781, which was filed on May 4, 2001, and which is incorporated herein by reference in its entirety.

In a particular embodiment, the solid support comprises a silicone elastomeric material such as, but not limited to, polydimethylsiloxane (“PDMS”). An advantage of silicone elastomeric materials is their flexible nature. In another particular embodiment, the solid support is a silicon wafer. The silicon wafer can be patterned and etched (see, e.g., G. Kovacs, 1998, Micromachined Transducers Sourcebook, Academic Press; M. Madou, 1997, Fundamentals of Microfabrication, CRC Press. The etched wafer can be used to cast the protein microarrays of the invention.

In one embodiment, a protein microarray used in methods herein includes a solid support that is a flat surface such as, but not limited to, a glass slide. Dense protein arrays can be produced on, for example, glass slides, such that assays for the presence, amount, and/or functionality of proteins can be conducted in a high-throughput manner.

Accordingly, in one embodiment, the protein microarray used in methods provided herein includes a plurality of proteins that are applied to the surface of a solid support, wherein the density of the sites at which protein are applied is at least 100 sites/cm², 1000 sites/cm², 10,000 sites/cm², 100,000 site/cm², 1,000,000 sites/cm², 10,000,000 sites/cm², 25,000,000 sites/cm², 10,000,000,000 sites/cm², or 10,000,000,000,000 sites/cm². Each individual protein sample is preferably applied to a separate site on the chip. The identity of the protein(s) at each site on the chip is/are known.

In another embodiment, the solid support has an array of wells. The use of microlithographic and micromachining fabrication techniques (see, e.g., co-pending U.S. application Ser. No. 09/849,781, filed on May 4, 2001, which is incorporated herein by reference in its entirety) can be used to create well arrays with a wide variety of dimensions ranging from hundreds of microns down to 100 nm or even smaller, with well depths of similar dimensions. In one embodiment, a silicon wafer is micromachined and acts as a master mold to cast wells of 400 μm diameter that are spaced 200 μm apart, for a well density of about 277 wells per cm², with individual well volumes of about 30 nl for 100 μm deep wells.

In another embodiment, microlithographic micromachining is used to fabricate wells 500 nm and 275 nm diameter, spaced 1 μm apart to yield well densities of over 44 million and over 61 million wells per cm² respectively. Higher densities are possible through closer spacing, as well as through smaller diameters.

In another embodiment, precision laser micromachining techniques can be used to directly fabricate mold structures out of acrylic with dimensions ranging from greater than 1.5 mm down to 500 μm, with well spacing of about 500 μm. Volumes of these wells are in the 50-500 nl range.

Accordingly, in one embodiment, the protein microarray comprises a plurality of wells on the surface of a solid support, wherein the density of wells is at least 100 wells/cm², 1000 wells/cm², 10,000 wells/cm², 100,000 wells/cm², 1,000,000 wells/cm², 10,000,000 wells/cm², 25,000,000 wells/cm², 10,000,000,000 wells/cm², or 10,000,000,000,000 wells/cm². The present invention contemplates variations of protein chips comprising a plurality of wells, which are disclosed, for example, in co-pending U.S. application Ser. No. 09/849,781, filed on May 4, 2001, and which is incorporated herein by reference in its entirety.

The present invention also contemplates variations in the shape, width-to-depth ratio and volume of wells in the protein microarray, which are disclosed, for example, in co-pending U.S. application Ser. No. 09/849,781, filed on May 4, 2001, and which is incorporated herein by reference in its entirety. Such shapes include, but are not limited to circular, oval, rectangular, square, etc. The wells can also have, for example, square, round V-shaped or U-shaped bottoms.

In one embodiment, the solid support comprises gold. In a preferred embodiment, the solid support comprises a gold-coated slide. In another embodiment, the solid support comprises nickel. In another preferred embodiment, the solid support comprises a nickel-coated slide. Solid supports comprising nickel are advantageous for purifying and attaching fusion proteins having a poly-histidine tag (“His tag”). In another embodiment, the solid support comprises nitrocellulose. In another preferred embodiment, the solid support comprises a nitrocellulose-coated slide.

Protein microarrays provided herein can be produced using compounds useful for derivatization of the protein microarray substrate. The proteins can be bound directly to the solid support, or can be attached to the solid support through a linker molecule or compound. The linker can be any molecule or compound that derivatizes the surface of the solid support to facilitate the attachment of proteins to the surface of the solid support. The linker may covalently or non-covalently bind the proteins or probes to the surface of the solid support. In addition, the linker can be an inorganic or organic molecule. In certain embodiments, the linker may be a silane, e.g., sianosilane, thiosilane, aminosilane, etc. The present invention contemplates compounds useful for derivatization of a protein chip, some of which are disclosed, for example, in co-pending U.S. application Ser. No. 09/849,781, which was filed on May 4, 2001, and which is incorporated herein by reference in its entirety.

Accordingly, proteins of microarrays used herein can be bound non-covalently to the solid support (e.g., by adsorption). Proteins that are non-covalently bound to the solid support can be attached to the surface of the solid support by a variety of molecular interactions such as, for example, hydrogen bonding, van der Waals bonding, electrostatic, or metal-chelate coordinate bonding. In a particular embodiment, proteins are bound to a poly-lysine coated surface of the solid support. In addition, as described above, in certain embodiments, the proteins are bound to a silane (e.g., sianosilane, thiosilane, aminosilane, etc.) coated surface of the solid support.

In addition, crosslinking compounds commonly known in the art, e.g. homo- or heterofunctional crosslinking compounds (e.g., bis[sulfosuccinimidyl]suberate, N-[gamma-maleimidobutyryloxy]succinimide ester, or 1-ethyl-3-[3-dimethylaminopropyl]carbodiimide), may be used to attach proteins to the solid support via covalent or non-covalent interactions.

In another embodiment, the proteins of the protein microarray are bound covalently to the solid support. For example, the proteins can be bound to the solid support by receptor-ligand interactions, which include interactions between antibodies and antigens, DNA-binding proteins and DNA, enzyme and substrate, avidin (or streptavidin) and biotin (or biotinylated molecules), and interactions between lipid-binding proteins and phospholipids (or membranes, vesicles, or liposomes comprising phospholipids).

Purified proteins can be placed on an array using a variety of methods known in the art. In one embodiment, the proteins are printed onto the solid support. In a further embodiment, the proteins are attached to the solid support using an affinity tag. Use of an affinity tag different from that used to purify the proteins is preferred, since further purification is achieved when building the protein array.

Accordingly, in a preferred embodiment, proteins of the protein microarray are expressed as fusion proteins having at least one heterologous domain with an affinity for a compound that is attached to the surface of the solid support. Suitable compounds useful for binding fusion proteins onto the solid support (i.e., acting as binding partners) include, but are not limited to, trypsin/anhydrotrypsin, glutathione, immunoglobulin domains, maltose, nickel, or biotin and its derivatives, which bind to bovine pancreatic trypsin inhibitor, glutathione-S-transferase, Protein A or antigen, maltose binding protein, poly-histidine (e.g., HisX6 tag), and avidin/streptavidin, respectively. For example, Protein A, Protein G and Protein A/G are proteins capable of binding to the Fc portion of mammalian immunoglobulin molecules, especially IgG. These proteins can be covalently coupled to, for example, a Sepharose® support to provide an efficient method of purifying fusion proteins having a tag comprising an Fc domain.

In a further embodiment, the proteins are bound directly to the solid support. In another further embodiment, the proteins are bound to the solid support via a linker. In a particular embodiment, the proteins are attached to the solid support via a His tag. In another particular embodiment, the proteins are attached to the solid support via a 3-glycidooxypropyltrimethoxysilane (“GPTS”) linker. In a specific embodiment, the proteins are bound to the solid support via His tags, wherein the solid support comprises a flat surface. In a preferred embodiment, the proteins are bound to the solid support via His tags, wherein the solid support comprises a nickel-coated glass slide.

Protein microarrays used in the methods provided herein are not limited in their physical dimensions and can have any dimensions that are useful. Preferably, the protein microarray has an array format compatible with automation technologies, thereby allowing for rapid data analysis. Thus, in one embodiment, the proteome microarray format is compatible with laboratory equipment and/or analytical software. In an illustrative example, the protein microarray is the size of a standard microscope slide. In another preferred embodiment, the protein chip is designed to fit into a sample chamber of a mass spectrometer. Illustrative protein arrays that can be used with the present invention are described, for example, in International Application Publication No. WO 02/092118 published Nov. 21, 2002.

Methods for Making and Parallel Processing Proteins

The methods, kits, and systems provided herein include protein arrays and protein microarrays, including high density protein microarrays (i.e. protein microarrays with arrayed proteins of greater than 100 proteins/cm². Typically, recombinant technologies are used to produce fusion proteins, which are isolated and analyzed in parallel using at least some automated processing steps. Accordingly, to obtain proteins to be immobilized on the microarray, known methods can be used for making and isolating viral, prokaryotic or eukaryotic proteins in a readily scalable format, amenable to high-throughput analysis. For example, methods include synthesizing and purifying proteins in an array format compatible with automation technologies. Therefore, in one embodiment, protein micrarrays for the invention a method for making and isolating eukaryotic proteins comprising the steps of growing a eukaryotic cell transformed with a vector having a heterologous sequence operatively linked to a regulatory sequence, contacting the regulatory sequence with an inducer that enhances expression of a protein encoded by the heterologous sequence, lysing the cell, contacting the protein with a binding agent such that a complex between the protein and binding agent is formed, isolating the complex from cellular debris, and isolating the protein from the complex, wherein each step is conducted in a 96-well format.

In a particular embodiment, eukaryotic proteins are made and purified in a 96-array format (ie., each site on the solid support where processing occurs is one of 96 sites), e.g., in a 96-well microtiter plate. In a preferred embodiment, the solid support does not bind proteins (e.g., a non-protein-binding microtiter plate). In certain embodiments, proteins are synthesized by in vitro translation according to methods commonly known in the art.

Any expression construct having an inducible promoter to drive protein synthesis can be used in accordance with the methods of the invention. Preferably, the expression construct is tailored to the cell type to be used for transformation. Compatibility between expression constructs and host cells are known in the art, and use of variants thereof are also encompassed by the invention.

Any host cell that can be grown in culture can be used to synthesize the proteins of interest. Preferably, host cells are used that can overproduce a protein of interest, resulting in proper synthesis, folding, and posttranslational modification of the protein. Preferably, such protein processing forms epitopes, active sites, binding sites, etc. useful for assays to characterize molecular interactions in vitro that are representative of those in vivo.

Accordingly, a eukaryotic cell (e.g., yeast, human cells) is preferably used to synthesize eukaryotic proteins. Further, a eukaryotic cell amenable to stable transformation, and having selectable markers for identification and isolation of cells containing transformants of interest, is preferred. Alternatively, a eukaryotic host cell deficient in a gene product is transformed with an expression construct complementing the deficiency. Cells useful for expression of engineered viral, prokaryotic or eukaryotic proteins are known in the art, and variants of such cells can be appreciated by one of ordinary skill in the art.

For example, the InsectSelect system from Invitrogen (Carlsbad, Calif., catalog no. K800-01), a non-lytic, single-vector insect expression system that simplifies expression of high-quality proteins and eliminates the need to generate and amplify virus stocks, can be used. A preferred vector in this system is pIB/V5-His TOPO TA vector (catalog no. K890-20). Polymerase chain reaction (“PCR”) products can be cloned directly into this vector, using the protocols described by the manufacturer, and the proteins can be expressed with N-terminal histidine tags useful for purifying the expressed protein.

Another eukaryotic expression system in insect cells, the BAC-TO-BAC™ system (INVITROGEN), can also be used. Rather than using homologous recombination, the BAC-TO-BAC™ system generates recombinant baculovirus by relying on site-specific transposition in E. coli. Gene expression is driven by the highly active polyhedrin promoter, and therefore can represent up to 25% of the cellular protein in infected insect cells.

In a particular embodiment, yeast cultures are used to synthesize eukaryotic fusion proteins. Fresh cultures are preferably used for efficient induction of protein synthesis, especially when conducted in small volumes of media. Also, care is preferably taken to prevent overgrowth of the yeast cultures. In addition, yeast cultures of about 3 ml or less are preferable to yield sufficient protein for purification. To improve aeration of the cultures, the total volume can be divided into several smaller volumes (e.g., four 0.75 ml cultures can be prepared to produce a total volume of 3 ml).

Cells are then contacted with an inducer (e.g., galactose), and harvested. Induced cells are washed with cold (ie., 4° C. to about 15° C.) water to stop further growth of the cells, and then washed with cold (i.e., 4° C. to about 15° C.) lysis buffer to remove the culture medium and to precondition the induced cells for protein purification, respectively. Before protein purification, the induced cells can be stored frozen to protect the proteins from degradation. In a specific embodiment, the induced cells are stored in a semi-dried state at −80° C. to prevent or inhibit protein degradation.

Cells can be transferred from one array to another using any suitable mechanical device. For example, arrays containing growth media can be inoculated with the cells of interest using an automatic handling system (e.g., automatic pipette). In a particular embodiment, 96-well arrays containing a growth medium comprising agar can be inoculated with yeast cells using a 96-pronger. Similarly, transfer of liquids (e.g., reagents) from one array to another can be accomplished using an automated liquid-handling device (e.g., Q-FILL™, Genetix, UK).

Although proteins can be harvested from cells at any point in the cell cycle, cells are preferably isolated during logarithmic phase when protein synthesis is enhanced. For example, yeast cells can be harvested between OD₆₀₀=0.3 and OD₆₀₀=1.5, preferably between OD₆₀₀=0.5 and OD₆₀₀=1.5. In a particular embodiment, proteins are harvested from the cells at a point after mid-log phase. Harvested cells can be stored frozen for future manipulation.

The harvested cells can be lysed by a variety of methods known in the art, including mechanical force, enzymatic digestion, and chemical treatment. The method of lysis should be suited to the type of host cell. For example, a lysis buffer containing fresh protease inhibitors is added to yeast cells, along with an agent that disrupts the cell wall (e.g., sand, glass beads, zirconia beads), after which the mixture is shaken violently using a shaker (e.g., vortexer, paint shaker).

In a specific embodiment, zirconia beads are contacted with the yeast cells, and the cells lysed by mechanical disruption by vortexing. In a further embodiment, lysing of the yeast cells in a high-density array format is accomplished using a paint shaker. The paint shaker has a platform that can firmly hold at least eighteen 96-well boxes in three layers, thereby allowing for high-throughput processing of the cultures. Further the paint shaker violently agitates the cultures, even before they are completely thawed, resulting in efficient disruption of the cells while minimizing protein degradation. In fact, as determined by microscopic observation, greater than 90% of the yeast cells can be lysed in under two minutes of shaking.

The resulting cellular debris can be separated from the protein and/or other molecules of interest by centrifugation. Additionally, to increase purity of the protein sample in a high-throughput fashion, the protein-enriched supernatant can be filtered, preferably using a filter on a non-protein-binding solid support. To separate the soluble fraction, which contains the proteins of interest, from the insoluble fraction, use of a filter plate is highly preferred to reduce or avoid protein degradation. Further, these steps preferably are repeated on the fraction containing the cellular debris to increase the yield of protein.

Proteins can then be purified from the protein-enriched supernatant using a variety of affinity purification methods known in the art. Affinity tags useful for affinity purification of fusion proteins by contacting the fusion protein preparation with the binding partner to the affinity tag, include, but are not limited to, calmodulin, trypsin/anhydrotrypsin, glutathione, immunoglobulin domains, maltose, nickel, or biotin and its derivatives, which bind to calmodulin-binding protein, bovine pancreatic trypsin inhibitor, glutathione-S-transferase (“GST tag”), antigen or Protein A, maltose binding protein, poly-histidine (“His tag”), and avidin/streptavidin, respectively. Other affinity tags can be, for example, myc or FLAG. Fusion proteins can be affinity purified using an appropriate binding compound (i.e., binding partner such as a glutathione bead), and isolated by, for example, capturing the complex containing bound proteins on a non-protein-binding filter. Placing one affinity tag on one end of the protein (e.g., the carboxy-terminal end), and a second affinity tag on the other end of the protein (e.g., the amino-terminal end) can aid in purifying full-length proteins.

In a particular embodiment, the fusion proteins have GST tags and are affinity purified by contacting the proteins with glutathione beads. In further embodiment, the glutathione beads, with fusion proteins attached, can be washed in a 96-well box without using a filter plate to ease handling of the samples and prevent cross contamination of the samples.

In addition, fusion proteins can be eluted from the binding compound (e.g., glutathione bead) with elution buffer to provide a desired protein concentration. In a specific embodiment, fusion proteins are eluted from the glutathione beads with 30 ml of elution buffer to provide a desired protein concentration.

For purified proteins that will eventually be spotted onto microscope slides, the glutathione beads are separated from the purified proteins. Preferably, all of the glutathione beads are removed to avoid blocking of the microarrays pins used to spot the purified proteins onto a solid support. In a preferred embodiment, the glutathione beads are separated from the purified proteins using a filter plate, preferably comprising a non-protein-binding solid support. Filtration of the eluate containing the purified proteins should result in greater than 90% recovery of the proteins.

The elution buffer preferably comprises a liquid of high viscosity such as, for example, 15% to 50% glycerol, preferably about 40% glycerol. The glycerol solution stabilizes the proteins in solution, and prevents dehydration of the protein solution during the printing step using a microarrayer.

Purified proteins are preferably stored in a medium that stabilizes the proteins and

prevents dessication of the sample. For example, purified proteins can be stored in a liquid of high viscosity such as, for example, 15% to 50% glycerol, preferably in about 40% glycerol. It is preferred to aliquot samples containing the purified proteins, so as to avoid loss of protein activity caused by freeze/thaw cycles.

The skilled artisan can appreciate that the purification protocol can be adjusted to control the level of protein purity desired. In some instances, isolation of molecules that associate with the protein of interest is desired. For example, dimers, trimers, or higher order homotypic or heterotypic complexes comprising an overproduced protein of interest can be isolated using the purification methods provided herein, or modifications thereof. Furthermore, associated molecules can be individually isolated and identified using methods known in the art (e.g., mass spectroscopy).

Methods for Making Protein Arrays

Protein microarrays used in the methods provided herein can be made by attaching (i.e. immobilizing) a plurality of proteins to a surface of a solid support, with each protein being at a different position on the solid support, wherein the plurality of proteins includes at least one representative protein for at least 1%, 2%, 3%, 4%, 5%, 10%, 20%, 30%, 40%, 50%, 60%, 70%, 80%, 90%, 95%, or 99% of the known genes in a species, wherein the protein is all protein isoforms and splice variants derived from a gene. In another aspect, protein microarrays provided herein include a plurality of proteins that were attached to a surface of a solid support, with each protein being at a different position on the solid support, wherein the plurality of proteins comprises at least 1%, 2%, 3%, 4%, 5%, 10%, 20%, 30%, 40%, 50%, 60%, 70%, 80%, 90%, 95%, or 99% of all proteins expressed in a species.

In another embodiment, the present invention provides a method for constructing a positionally addressable array that includes the step of attaching a plurality of proteins to a surface of a solid support, with each protein being at a different position on the solid support, wherein the plurality of proteins comprises at least 1, 2, 3, 4, 5, 10, 20, 30, 40, 50, 100, 200, 500, 1000, 1500, 2000, 2500, 3000, 4000, 5000, 6000, 7000, 8000, 9000, 10,000, 100,000, 500,000 or 1,000,000 protein(s), which in certain aspects are expressed in a species.

In preferred aspects, as further disclosed in the section entitled “Methods for Making and Parallel Processing of Proteins,” protein microarrays can be produced using recombinant technologies and multiple channel formats, such that many protein production, isolation, and quality control reactions are performed in parallel, typically using robotics and multi-channel pipetors. In illustrative aspects, the proteins are recombinant fusion proteins.

Accordingly, provided herein as a separate embodiment, is a method for large-scale manufacturing a protein array product. The method includes expressing a plurality of proteins tagged with a purification tag, for example Glutathione-S-Transferase (GST) in a recombinant organism. Culturing of recombinant organisms expressing the tagged proteins for the microarray, as well as protein processing, can be carried out in multi-well boxes such as 96-well boxes. The proteins are isolated by lysing cells and processing includes an affinity column that binds the purification tag. Isolated proteins are then spotted onto a substrate to product the microarray.

In an illustrative example, the substrate is a nitrocellulose substrate. In one aspect, the proteins are expressed in a eukaryotic organism such as yeast. In another example, the proteins are expressed using an in vitro expression system. Processing of the recombinant cells and proteins is carried out in a semi-automated or automated manner. In certain aspects, all steps are carried out at 4C. In one aspect, the microarrays include at least 1 ng/ul of each protein and the microarray includes between 100 and 1,000,000 different proteins, or between 100 and 50,000 different proteins. In one example, microarrays are only sold to customers if they include protein of a concentration more than 1 ng/ul.

As illustrated in Example 4, the method provides consistent production of protein microarrays that have a median concentration of 1 ug/ml or greater. Accordingly, in another aspect is a method for producing at least 100 microarrays, wherein each microarray contains at least 100, 200, 250, 500, 1000, 2000, 2500, 5000, 10,000 different spotted proteins and wherein the median concentration of each protein on the microarray is at least 0.5 ng/ul, 1 ng/ul, 2 ng/ul, 5 ng/ul, 10 ng/ul, 100 ng/ul, or 1 ug/ul. In an illustrative example the protein concentration is at least 1 ng/ul.

Protein microarrays used in methods provided herein can be produced by attaching a plurality of proteins to a surface of a solid support, with each protein being at a different position on the solid support, wherein the protein comprises at least one tag. In certain preferred aspects, the proteins on the solid support include two tags, other than for example, control proteins on the solid support, which can include no tags, one tag, or more than two tags. The advantages of using double-tagged proteins include the ability to obtain highly purified proteins, as well as providing a streamlined manner of purifying proteins from cellular debris and attaching the proteins to a solid support. The tag can be for example, a glutathione-S-transferase tag (“GST tag”), a poly-histidine tag (His tag”), or a biotin tag. The biotin tag can be associated with a protein in vivo or in vitro. Where in vivo biotinylation is used, a peptide for directing in vivo biotinylation can be fused to a protein. For example, a Bioease™ tag can be used. In certain aspects, a biotin tag is used for protein immobilization on a protein microarray substrate and/or to isolate a recombinant fusion protein before it is immobilized on a substrate at a positionally addressable location. In a particular embodiment, the first tag is a glutathione-S-transferase tag (“GST tag”) and the second tag is a poly-histidine tag (“His tag”). In a further embodiment, the GST tag and the His tag are attached to the amino-terminal end of the protein. Alternatively, the GST tag and the His tag are attached to the carboxy-terminal end of the protein.

In yet another embodiment, the GST tag is attached to the amino-terminal end of the protein. In a further embodiment, the His tag is attached to the carboxy-terminal end of the protein. In yet another embodiment, the His tag is attached to the amino-terminal end of the protein. In a further embodiment, the GST tag is attached to the carboxy-terminal end of the protein.

In yet another embodiment, the protein comprises a GST tag and a His tag, and neither the GST tag nor the His tag is located at the amino-terminal or carboxy-terminal end of the protein. In a specific embodiment, the GST tag and His tag are located within the coding region of the protein of interest; preferably in a region of the protein not affecting the binding domain of interest.

In one embodiment, the first tag is used to purify a fusion protein. In another embodiment, the second tag is used to attach a fusion protein to a solid support. In a specific further embodiment, the first tag is a GST tag and the second tag is a His tag.

The protein preferably is a fusion protein such that the heterologous sequence comprises the coding region for the protein of interest and sequences encoding a tag, such as an affinity tag. Such tags can be useful for monitoring the protein, separating the fusion protein from cellular debris and contaminating reagents, and/or attaching the protein to a protein microarray of the invention.

Examples of inducers include, but are not limited to, galactose, enhancer-binding proteins, and other transcription factors. In one embodiment, galactose is contacted with a regulatory sequence comprising a galactose-inducible GALI promoter.

A binding agent that can be used in accordance with the invention includes, but is not limited to, a glutathione bead, a nickel-coated solid support, and an antibody. In one embodiment, the complex comprises a fusion protein having a GST tag bound to a glutathione bead. In another embodiment, the complex comprises the a fusion protein having a His tag bound to a nickel-coated solid support. In yet another embodiment, the complex comprises the protein of interest bound to an antibody and, optionally, a secondary antibody.

Protein microarrays used in the methods provided herein can be used to assay the presence, amount, and/or functionality of proteins present in at least one sample. Using the protein microarrays chemical reactions and assays in a large-scale parallel analysis can be performed to characterize biological states or biological responses, and determine the presence, amount, and/or biological activity of proteins. Accordingly, protein microarrays can be used to assay for essentially all protein-protein interactions in a cell, tissue, organ, system, or organism. Biological activity that can be determined using a protein microarray used in the methods of the invention includes, but is not limited to, enzymatic activity (e.g., kinase activity, protease activity, phosphatase activity, glycosidase, acetylase activity, and other chemical group transferring enzymatic activity), nucleic acid binding, hormone binding, etc. High density and small volume chemical reactions can be advantageous for the protein microarrays used in the methods and kits provided herein.

Upon contacting the proteins of a protein microarray with one or more probes, protein-probe interactions can be assayed using a variety of techniques known in the art. For example, the protein microarray can be assayed using standard enzymatic assays that produce chemiluminescence or fluorescence. Various protein modifications can be detected by, for example, photoluminescence, chemiluminescence, or fluorescence using non-protein substrates, enzymatic color development, mass spectroscopic signature markers, or amplification of oligonucleotide tags.

The probe is labeled or tagged with a marker so that its binding can be detected, directly or indirectly, by methods commonly known in the art. Furthermore, tagged polypeptides and proteins are used in methods provided herein for determining protein concentrations. Finally, tagged polypeptides and proteins can be used in control methods provided herein for detecting labeling of a polypeptide. Any art-known marker may be used, including but not limited to tags such as epitope tags, haptens, and affinity tags, antibodies, labels, etc., providing that it is not the same as the affinity tag or reagent used to attach the protein(s) of the protein microarray to the solid substrate of the chip. For example, if biotin is used as a linker to attach proteins to a protein microarray array, then another tag not present in the protein(s) of the protein microarray, e.g., His or GST, is used to label the probe and to detect a protein-probe interaction. In certain embodiments, a photoluminescent, chemiluminescent, fluorescent, or enzymatic tag is used. In other embodiments, a mass spectroscopic signature marker is used. In yet other embodiments, an amplifiable oligonucleotide, peptide or molecular mass label is used.

Modification or binding of proteins on the protein microarray can be detected by, for example, 1) using radioactively labeled ligand followed by autoradiography and/or phosphoimager analysis; 2) binding of hapten, which is then detected by a fluorescently labeled or enzymatically labeled antibody or high-affinity hapten ligand such as biotin or streptavidin; 3) mass spectrometry; 4) atomic force microscopy; 5) fluorescent polarization methods; 6) infrared red labeled compounds or proteins; 7) amplifiable oligonucleotides, peptides or molecular mass labels; 8) stimulation or inhibition of the protein's enzymatic activity; 9) rolling circle amplification-detection methods (Hatch et al., 1999, “Rolling circle amplification of DNA immobilized on solid surfaces and its application to multiplex mutation detection”, Genet. Anal. 15:35-40); 10) competitive PCR (Fini et al., 1999, “Development of a chemiluminescence competitive PCR for the detection and quantification of parvovirus B 19 DNA using a microplate luminometer”, Clin Chem. 45:1391-6; Kruse et al., 1999, “Detection and quantitative measurement of transforming growth factor-beta1 (TGF-beta1) gene expression using a semi-nested competitive PCR assay”, Cytokine 11:179-85; Guenthner and Hart, 1998, “Quantitative, competitive PCR assay for HIV-1 using a microplate-based detection system”, Biotechniques 24:810-6); 11) colorimetric procedures; and 12) biological assays (e.g., for virus titers).

In a particular embodiment, protein-probe interactions are detected by direct mass spectrometry. In a further embodiment, the identity of the protein and/or probe is determined using mass spectrometry. For example, one of more probes that have bound to a protein on the protein microarray can be dissociated from the array, and identified by mass spectrometry (see, e.g., WO 98/59361). In another example, enzymatic cleavage of a protein on the protein microarray can be detected, and the cleaved protein fragments or other released compounds can be identified by mass spectrometry.

In one embodiment, each protein on the protein microarray is contacted with a probe, and the protein-probe interactions are detected and quantified. In another embodiment, each protein on the protein microarray is contacted with multiple probes, and the protein-probe interaction is detected and quantified. For example, the protein microarray can be simultaneously screened with multiple probes including, but not limited to, complex mixtures (e.g., cell extracts), intact cellular components (e.g., organelles), whole cells, and probes pooled from several sources. The protein-probe interactions are then detected and quantified. Useful information can be obtained from assays using mixtures of probes due, in part, to the positionally addressable nature of the arrays of the present invention, i.e., via the placement of proteins at known positions on the protein chip, the protein to which the probe binds (“interactor”) can be characterized.

One of ordinary skill in the art can appreciate many different embodiments for assaying various cellular interactions by using probes to screen the protein microarrays of the invention. For example, multiple sequential screens of a protein microarray with various probes can define all proteins involved in a particular signal transduction pathway or in a specific metabolic pathway. Moreover, these assays can be useful for diagnostic, prognostic and/or therapeutic purposes.

In accordance with the methods of the invention, a probe can be a cell, cell membrane, subcellular organelles, protein-containing cellular material, protein, oligonucleotide, polynucleotide, DNA, RNA, small molecule (i.e., a compound with a molecular weight of less than 500), substrate, drug or drug candidate, receptor, antigen, steroid, phospholipid, antibody, immunoglobulin domain, glutathione, maltose, nickel, dihydrotrypsin, lectin, or biotin.

Probes can be biotinylated for use in contacting a protein array so as to detect protein-probe interactions. Weakly biotinylated proteins are more likely to maintain the biological activity of interest. Thus, a gentler biotinylation procedure is preferred so as to preserve the protein's binding activity or other biological activity of interest. Accordingly, in a particular embodiment, probe proteins are biotinylated to differing degrees using a biotin-transferring compound (e.g., Sulfo-NHS-LC-LC-Biotin; PIERCE™ Cat. No. 21338, USA).

In addition, the probe can be an enzyme substrate or inhibitor. For example, the probe can be a substrate or inhibitor of an enzyme such as, but not limited to, kinases, phosphatases, proteases, glycosidases, acetylases, and other group transferring enzymes. After incubation of proteins on a chip with combinations of nucleic acid or protein probes, the bound nucleic acid or protein probes can be identified, for example, by mass spectrometry (Lakey et al., 1998, “Measuring protein-protein interactions”, Curr Opin Struct Biol. 8:119-23).

Accordingly, various cellular responses to interaction with the proteins on a protein microarray can be assayed by probing with whole cells. For example, a protein microarray can be contacted with lymphocytes and assayed for lymphocyte activation by various means including, but not limited to, detecting antibody synthesis, detecting or measuring incorporation of ³H-thymidine, labeling cell surface molecules with antibodies to identify molecules induced or suppressed by antigen recognition and activation (e.g., CD23, CD38, IgD, C3b receptor, IL-2 receptor, transferrin receptor, membrane class II MHC molecules, PCA-1 molecules, HLA-DR), and identifying expressed and/or secreted cytokines.

In another example, mitogens for a specific cell-type can be determined by incubating a cell with a protein microarray. Mitotic activity can be determined, for example, by detecting or measuring incorporation of ³H-thymidine by a cell. Cells can be of the same cell type (i.e., a homogeneous population) or can be of different cell types.

In another example, differentiation factors for a specific cell-type can be determined by incubating a cell with a protein microarray. Differentiation of a cell can be determined, for example, by visual inspection, detection of cell-surface differentiation markers using marker-specific antibodies, or identification of secreted differentiation markers.

In another example, apoptotic factors for a specific cell-type can be determined by incubating a cell with a protein microarray. Apoptosis can be assayed, for example, by visual inspection, detection of cell-surface apoptotic markers using marker-specific antibodies, or identification of secreted markers or other cellular components released into the media.

In another example, the secretory response of a cell to a protein on a protein microarray can be assayed by incubating a cell with a protein microarray of the invention. Secreted proteins and other cellular compounds can be assayed, for example, by detecting the released compounds in the media.

In another example, the ability of a protein on a protein microarray to mediate cell aggregation can be assayed, for example, by incubating one or more cells with a protein microarray of the invention, and assaying for aggregation. Also, a protein's ability to mediate an affinity to extracellular matrix can be assayed by, for example, incubating a cell and extracellular matrix components with a protein microarray, and assaying for enhanced affinity of the cell or the extracellular matrix component with a protein on the chip. Interactors identified in such assays can have a role in, for example, cancer, cell migration, synaptogenesis, dendritic growth, process extension, or axonal elongation.

In yet another example, the effect of proteins of a protein microarray of the invention on ion transport, or other small molecule transport (e.g., ATP), can be determined. For example, the probe cells can be pre-loaded with a radioactively labeled ion or other small molecule, and incubated with a protein microarray of the invention. Retention or release of the radioactive label can be measured at different time points after contacting the cells with the proteins of the proteome array. Alternatively, ion transport can be detected and characterized using electrophysiological techniques known in the art.

In yet another example, cellular uptake and/or processing of proteins on the protein microarrays can be assayed by, for example, incubating a cell with a protein microarray having radioactively or fluorescently labeled proteins on the chip, and measuring the increase or decrease in signal on the protein microarray, or measuring uptake of labeled protein by the cell.

Alternatively, a protein microarray of the invention can be incubated with a cell and a labeled compound of interest, such that cellular uptake and/or processing of the compound by the cell is detected and/or measured.

Interactions of small molecules (i.e., compounds smaller than MW=500) with the proteins on a protein microarray also can be assayed in a cell-free system by probing with small molecules such as, but not limited to, ATP, GTP, cAMP, phosphotyrosine, phosphoserine, and phosphothreonine. Such assays can identify all proteins in a species that interact with a small molecule of interest. Small molecules of interest can include, but are not limited to, pharmaceuticals, drug candidates, fungicides, herbicides, pesticides, carcinogens, and pollutants. Small molecules used as probes in accordance with the methods of the invention preferably are non-protein, organic compounds.

In another embodiment, essentially all receptors for a particular ligand, or class of ligands, in a species can be identified by contacting a receptor of interest with a protein microarray. Alternatively, essentially all ligands in a species that are identified by a particular receptor or receptor family of interest can be identified by contacting a receptor of interest with a protein microarray of the invention. In another embodiment, essentially all proteins in a species, capable of inhibiting or blocking formation of a particular receptor-ligand complex, can be identified by contacting a receptor and its ligand with a protein microarray of the invention, and determining whether receptor-ligand interaction is inhibited as compared with the degree of receptor-ligand interaction in the absence of the protein on the chip. Detection of receptor-ligand interaction and identification of the ligand interactors can be accomplished using methods known in the art.

In another embodiment, essentially all kinase targets in a species can be identified by, for example, contacting a kinase with a protein microarray of the invention, and in the presence of labeled phosphate, detecting phosphorylated interactors using methods known in the art. Alternatively, essentially all kinases in a species can be identified by contacting a substrate that can be phosphorylated with a protein microarray of the invention, and assaying the presence and/or level of phosphorylated substrate by, for example, using an antibody specific to a phosphorylated amino acid. In another embodiment, essentially all kinase inhibitors in a species can be identified by contacting a kinase and its substrate with a protein microarray of the invention, and determining whether phosphorylation of the substrate is reduced as compared with the level of phosphorylation in the absence of the protein on the chip.

Detection methods for kinase activity are known in the art, and include, but are not limited to, the use of radioactive labels (e.g., ³³P-ATP and ³⁵S-g-ATP) or fluorescent antibody probes that bind to phosphoamino acids.

Similarly, assays can be conducted to identify all phosphatases, and inhibitors of a phosphatase, in a species. For example, whereas incorporation into a protein of radioactively labeled phosphorus indicates kinase activity in one assay, another assay can be used to measure the release of radioactively labeled phosphorus into the media, indicating phosphatase activity.

The protein microarrays used in methods of the invention can also be used to distinguish different cell types (either morphological or functional) by, for example, contacting a protein microarray with cells or cell extracts representing different populations of cells, and comparing the patterns of protein-probe interactions on the protein microarray. In particular, cell extracts representing two different populations of cells can be labeled with two different labels, mixed, and then used to contact the protein microarrays used in methods of the invention. The ratio of the two labels at every protein location of the protein microarray can be used to determine whether any protein interaction is increased or decreased in a particular population of cells. This information can be compared to known interactions for various different cell lines. This approach also can be used to characterize, for example, different stages of the cell cycle, disease states, altered physiologic states (e.g., hypoxia), physiological state before or after treatment (e.g., drug treatment), metabolic state, stage of differentiation, developmental stage, response to environmental stimuli (e.g., light, heat), response to environmental toxins (e.g., pesticides, herbicides, pollution), cell-cell interactions, cell-specific protein expression, and disease-specific protein expression.

Developmental profiles of protein-protein interactions can be used to characterize signal transduction pathways, metabolic pathways, etc. involved at every development stage and elucidate transitions between developmental stages. The wealth of information provided by such studies can be used to identify drug targets for each stage, and/or tailor treatment regimens during the course of a disease.

The protein microarrays used in the methods of the invention can be incubated with cell extracts to characterize a particular cell type, response to a stimulus, or physiological state. Accordingly, in exemplary embodiments, a protein microarray of the invention can be contacted with a cell extract from cells treated with a compound (e.g., a drug), or from cells at a particular stage of cell differentiation (e.g., pluripotent), or from cells in a particular metabolic state (e.g., mitotic), and assayed for kinase, protease, glycosidase, actetylase, phosphatase, and/or other transferase activity, for example.

The pattern of protein-probe interactions on the protein microarray can thereby provide a “signature” or “fingerprint” characteristic of the biological state. For example, the results obtained from such assays, comparing for example, cells in the presence or absence of a drug, or cells at several differentiation stages, or cells in different metabolic states, can provide a signature of each condition, and can provide information regarding the physiologic changes in the cells under the different conditions.

Clearly, by screening a species's proteome using a plurality of probes (e.g., known mixtures of probes, cellular extracts, subcellular organelles, cell membrane preparations, whole cells, etc.), the resulting analysis of protein-probe interactions can form the basis of identifying a “fingerprint” or “signature” of the a cell-type or physiological state of a cell, tissue, organ or system. Such information can be useful for diagnosis, prognosis, drug testing, and drug discovery, for example.

Accordingly, the protein microarrays of the invention can be used to determine a drug's interactions with proteins on the chip. Alternatively, the protein microarrays of the invention can be used to characterize a drug's effects on complex protein mixtures such as, for example, whole cells, cell extracts, or tissue homogenates. For example, a protein microarray can be contacted with a complex protein mixture and assayed for altered interactions of the protein mixture with the proteins on the chip when compared in presence or absence of drug.

The net effect of a drug can thereby be analyzed by screening one or more protein microarrays with drug-treated cells, tissues, or extracts, which then can provide a “signature” for the drug-treated state, and when compared with the “signature” of the untreated state, can be of predictive value with respect to, for example, potency, toxicity, and side effects. Furthermore, time-dependent effects of a drug can be assayed by, for example, adding the drug to the cell, cell extract, tissue homogenate, or whole organism, and applying the drug-treated cells or extracts, prepared at various timepoints of the treatment, to a protein microarray. Such assays can be useful for diagnosis or prognosis of a disease.

In particular, the protein microarrays used in the methods of the invention can be useful for characterizing a mode of action of a drug, determining drug specificity, predicting drug toxicity, and for drug discovery. For example, the identity of proteins that bind to a drug, and their relative affinities, can be assayed by incubating a protein microarray with a drug or drug candidate under different assay conditions, determining drug specificity by determining where on the array the drug bound, and measuring the amount of drug bound by each different protein.

The protein microarrays used in the methods of the invention can be used to determine a disease state by, for example, contacting a protein microarray with diseased cells, cell extracts or tissue homogenates from diseased tissue, or body fluids from a patient suffering from a disease, and comparing the pattern of protein-probe interactions on the protein microarray with that of a healthy counterpart. Such assays can provide a “signature” for the disease state, and when compared with the “signature” of the healthy state, can be of predictive value with respect to, diagnosis or prognosis of the disease. Furthermore, stages of a disease can be characterized by, for example, assaying biological preparations on the protein microarray at various stages of the disease.

Bioassays in which a biological activity is assayed, rather than binding assays, can also be conducted out on the same protein microarray, or on an identical second chip. Thus, these types of assays using the protein chips of the invention are useful for studying drug specificity, predicting potential side effects of drugs, and classifying drugs.

Further, protein microarrays used in methods of the invention are suitable for screening complex libraries of drug candidates. Specifically, the proteins on the chip can be incubated with the library of drug candidates, and then the bound components can be identified, e.g., by mass spectrometry, which allows for the simultaneous identification of all library components that bind preferentially to specific subsets of proteins, or bind to several of the proteins on the chip. Additionally, the relative affinity of the drug candidates for the different proteins in the array can be determined.

Moreover, the protein chips used in methods of the present invention can be probed in the presence of potential inhibitors, catalysts, modulators, or enhancers of an observed interaction, enzymatic activity, or biological response. Using a protein microarray of the present invention, such strategic screens can identify proteins expressed in a species that, for example, block the binding of a drug, inhibit of viral infection, exhibit bacteriostatic activity, exhibit anti-fungal activity, ameliorate parasitic infection, or physiological effectors to specific categories of proteins.

Enzymatic reactions can be performed and enzymatic activity measured using the protein microarrays of the present invention. In a specific embodiment, compounds that modulate the enzymatic activity of a protein or proteins on a chip can be identified. For example, changes in the level of enzymatic activity can be detected and quantified by incubating a compound or mixture of compounds with an enzymatic reaction mixture, thereby producing a signal (e.g., from substrate that becomes fluorescent upon enzymatic activity). Differences between the presence and absence of a test compound can be characterized. Furthermore, the differences in a compound's effect on enzymatic activities can be detected by comparing their relative effect on samples within the protein microarray and between chips.

Kits.

In another embodiment, provided herein is a kit, including a test protein microarray comprising at least 10 different polypeptides; and a control protein microarray that is different than the test protein microarray, wherein the control protein microarray includes a first specific binding pair member that binds to a first detectable label. The test protein microarry typically is a high-density protein microarray that includes at least 100 proteins/cm². The proteins on the test array typically include at least 10, 25, 50, 75, 100, 200, 250, 500, 1000, 2000, 2500, 5000, 10000, or 20,000 different polypeptides and/or proteins. In certain aspects the array has a maximum of 50,000 polypeptides and/or proteins. The proteins on the test array, in certain illustrative aspects, are related proteins. Related proteins are typically proteins of the same protein family, enzyme class, biological pathway, species, or related group of species, such as the same genera.

In one aspect, the first detectable label is an epitope tag. In this aspect, the first specific binding pair member can be, for example, an antibody that binds the epitope tag.

The control microarray is used to verify a labeling protocol and/or probing conditions. The test protein microarray is used to identify a protein-protein interaction or an enzymatic reaction. In certain aspects, the label is biotin and the first specific binding pair member that binds to biotin is an antibody that binds biotin. A specific example of a kit of the invention is provided in Yeast ProtoArray™ PPI Kit manual provided as an Attachment and referenced in the Examples section herein.

The test protein microarray and/or the control microarry can further include a first specific binding pair member of a second specific binding pair. Furthermore, the kit can include a second SBP member of the second SBP. The second specific binding pair member can be a control polypeptide labeled with the first detectable label, in certain illustrative examples, with one copy of the first detectable label. For example, the test protein microarray and/or the control microarray can include a polypeptide such as calmodulin, as an example of a first SBP member of a second SBP, and the kit can include a control polypeptide that is a calmodulin binding protein, such as calmodulin kinase. The calmodulin kinase can be labeled with the first detectable label or instructions can be provided such that a customer that purchases the kit can label the calmodulin-binding protein with the first detectable label.

In certain aspects, the control polypeptide, for example, calmodulin kinase, is provided in the kit labeled with one copy of the first detectable label. Thus, although, for example, an antibody against the label is provided spotted on the control microarray, probing this microarray with the control polypeptide followed by a probe that binds to the label and is labeled with a second detectable label will result in a positive signal at the spot of binding pair member that binds the control polypeptide, but will not result in a detectable signal at the location of the antibody.

In certain aspects the kit includes 2 copies of the test protein microarray and/or 2 control protein microarrays. Two test protein microarrays can be included in order to allow two experiments to be performed according to the methods provided herein, but under different experimental conditions. Two control protein microarrays can be included, for example wherein a first control protein microarray comprises a first SBP member of a first SBP, such as an antibody against the label, to validate labeling. A second control protein microarray can include a first SBP member of a second SBP. The kit can also include a second SBP member of the second SBP. The second SBP member of the second SBP can optionally be labeled with the first detectable label. Therefore, the second SBP member of the second SBP can be used to probe a control protein array in order to assure that a probing reaction is performed successfully before a test protein array is used.

The kit can further include a second detectably labeled molecule that includes a second label, wherein the second detectably labeled molecule binds to the first detectable label. For example, where the label is biotin, the second detectably labeled molecule can be fluorescently labeled streptavidin. In certain preferred aspects, the fluorescent label is AlexaFluor.

The kit can also include a protein labeling reagent that includes the first detectable label. For example, wherein the first detectable label is biotin, the kit can include a biotinylation reagent as well as other related reagents for biotinylating a protein (as illustrated in the Examples provided herein). Furthermore, the kit can include a purification module for removing free label after a labeling reaction. The kit can also include reagents and compositions for assessing labeling of the first specific binding pair member, which is typically the probe. The kit can further include reagents for blocking and washing steps during a method provided herein.

Virtually any substrate for a protein microarray, as discussed in further detail herein and/or as known in the art, can be used for the protein microarrays provided in a kit provided herein. For example, in one aspect, the kit is used to detect protein-protein interactions and the test protein microarray includes glass substrate that is overlaid with a protein-binding filter, such as a nitrocellulose filter.

In certain aspects, the kit can be used to test for enzymatic activity. In these aspects, the kit can further include an enzyme substrate. Furthermore, in these aspects, the test protein microarray can include a functionalized glass substrate.

The kit in some examples, can further include a test microarray and/or a control microarray having a series of spots derived from solutions comprising different known concentrations of a control protein that includes a tag. The series can be presented in duplicate, triplicate, quadruplicate, or even more copies. Concentrations of proteins spotted on the arrays can be determined by creating a standard curve using the series of addressable spots with different known concentrations of the control protein that includes the tag The tag can be a purification tag, such as, for example, glutathione S-transferase.

Systems.

In certain embodiments, provided herein is a system that includes a protein microarray comprising at least 100 proteins/cm² and a microarray identifier; a control protein microarray comprising a first specific binding pair member that binds to a first detectable label; and an internet portal comprising information regarding the identification and concentration of proteins on the microarray based on the identity of the array provided by the microarray identifier. The components of the system are discussed in further detail herein. The Internet portal typically provides automated ordering of a protein array-related product or service based on identification of a target protein on the test protein array.

The internet portal can include links to computer programs for carrying out various aspects of the methods provided herein. For example, known methods, referred to herein as image analysis functions, can be provided as links on the internet portal and used to identify whether a signal is positive. For example, the functions can be provided by downloadable software available on the portal, such as the software disclosed in the ProtoArray™ Prospector™ manual, incorporated by reference in its entirety, available on the worldwide web at Invitrogen.com. The positive signal can indicate, for example, an interaction between an immobilized protein array protein and a probe, or an enzymatic modification of an immobilized protein array protein by an enzyme used to probe the protein array. For example, the positive signal can identify a protein that is phosphorylated by a kinase used to probe the protein array. Signals can be in any measurable form including, but not limited to, visible light, ultraviolet radiation, infrared radiation, X-rays, fluorescence, and colorimetric visualization. In certain aspects, signals are detected by mass spectrometry. Signals can be produced by fluorescence and are arranged in a grid pattern on an array. As such, a signal can be assigned a positional coordinate with respect to row and column. The rows and columns can be of any width.

The first step in filtering signals is to calculate the local foreground and background signals for each spot. The local foreground signal is emitted from the actual spot, whereas the local background signal is emitted from the area immediately bordering to the spot. The net signal, which is the local foreground signal minus the local background signal, is used in all further calculations. The local foreground and background signals can be identified by software such as GENEPIX™. In certain embodiments of the present invention,

However, variations between chips, which can represent, for example, different lipid-binding experiments, and local variations on the chip (due to unequal diffusion of substrates, for example) can result in further fluctuation of the net signal intensity, resulting in different net signal distributions for different chips. To correct the variation between chips, the net signals from different chips need to be scaled into a common range. One of the several chips is chosen as a reference and the goal is to scale the net signal distributions of each chip to the range and shape of the net signal distribution of the reference chip.

For example, the lower quartile, median, and upper quartile values of the net signal distribution of each chip can be computed. Then, for each chip, the median net signal is subtracted from the net signal of each spot. Furthermore a scaling factor is computed for each chip, which is equal to the ratio of the difference between the upper and lower quartile of the specific chip and the difference between the upper and lower quartile of the reference chip. This implies that the scaling factor for the reference chip is equal to one. Then the net signals on each chip are multiplied by the chip-specific scaling factor to calculate the scaled net signals.

To correct for local variations on an array, a “neighborhood subtraction” for each spot can be performed. For example, the neighborhood region can be defined as a region of two rows above and below, as well as two columns to the left and right of a signal spot. The median signal of this region is then subtracted from the spot signal to calculate an excess signal relative to the neighborhood of the spot. Preferably, the number of spots of high signal strength in any neighborhood region is sufficient low, such that the median value is not significantly affected and is a good representation of the background signal in the neighborhood region.

Applying the neighborhood subtraction to the scaled net signals yields the scaled excess signals. In the next step, parallel samples are compared with respect to their scaled excess signals. If the difference between the average of the scaled excess signal of the two parallel samples and the scaled excess signal of one of the parallel samples is greater than three times the standard deviation of the error of the scaled excess signal, the spots belonging to the two parallel samples are excluded from further analysis. The remaining spots and their scaled excess signal then represent the set of filtered signals. A positive signal typically indicates a protein-probe interaction.

Highly Sensitive Method for Detecting Protein Interactions on a Microarray

Provided in another embodiment herein is a highly sensitive method for detecting protein interactions on a microarray, the method includes contacting proteins immobilized on a microarray with a labeled test probe (i.e. a labeled probe protein), and detecting the label, wherein a detected protein includes between 1 pg and 100 pg of protein immobilized on the array, and wherein the probe is used at a concentration of less than 1 ug. As illustrated in Example 1, the method provides detection of as little as 1 pg of protein on a protein microarray. Furthermore, submicrogram quantities of probe protein can be used for this detection. In certain aspects, the probe is a biotin-labeled probe. In one illustrative example, one to five biotins are attached to the test protein. In an illustrative aspect, the label is a fluorescent label. In one illustrative example, the fluorescent label is Alexa fluor (Invitrogen).

The present invention may be better understood by reference to the following non-limiting Examples, which are provided as exemplary of the invention. The following examples are presented in order to more fully illustrate the preferred embodiments of the invention. They should in no way be construed, however, as limiting the broad scope of the invention.

### EXAMPLE 1

### Yeast Protoarray PPI Kit

The Yeast ProtoArray™ PPI (Protein-Protein Interaction) Kit is shipped as detailed below. Upon receipt, store as indicated. For details on each component, see below.

All kit components are stable for 6 months when stored properly.

| Kit Contents | Shipping | Storage |
|---|---|---|
| Yeast ProtoArray ™ PPI Proteome Microarray | Blue ice | −20° C. |
| Yeast ProtoArray ™ PPI Control Microarray | Blue ice | −20° C. |
| ProtoArray ™ Buffers Module A | Dry ice | −20° C. |
| ProtoArray ™ Buffers Module B | Blue ice | 4° C. |
| ProtoArray ™ Mini-Biotinylation Module | Dry ice | −20° C. |
| ProtoArray ™ Biotinylation Purification Module | Blue ice | 4° C. |
| ProtoArray ™ Biotinylation Assessment Module | Blue ice | 4° C. |

Yeast ProtoArray™ PPI Proteome Microarray Box contains a mailer with PPI Microarrays 2 yeast proteome microarrays and Yeast ProtoArray™ PPI Control Microarray Box contains a mailer with 2 control microarrays.

The following components are included in the ProtoArray™ Buffers Module A.

Sufficient Buffers are included to perform 4 microarray screening experiments.

| Component | Composition | Amount |
|---|---|---|
| Bovine Serum Albumin (BSA) | 30% in 0.85% NaCl | 30 ml |
| DTT | 1 M in deionized water | 400 μl |
| Array Control Protein | 0.5 μg/μl in PBS (phosphate | 20 μl |
| (Biotinylated Calmodulin kinase) | buffered saline), pH 7.4 | |

The following components are included in the ProtoArray™ Buffers Module B. Store at 4° C. Protect Streptavidin-Alexa Fluor® 647 conjugate from light. Sufficient Buffers are included to perform 4 microarray screening experiments.

| Component | Composition | Amount | |
|---|---|---|---|
| ProtoArray ™ Blocking Buffer | 10× PBS, pH 7.4 | 12 | ml |
| (10×) | 1% Tween 20 | | |
| ProtoArray ™ Probe Buffer | 5× PBS, pH 7.4 | 175 | ml |
| (5×) | 0.25% Triton X-100 25% | | |
| | Glycerol | | |
| MgCl₂ | 1 M in deionized water | 4 | ml |
| Streptavidin-Alexa Fluor ® 647 | 2 mg/ml in PBS, pH 7.2 | 30 | μl |
| Conjugate | with 5 mM sodium azide | | |

| HybriSlip ™ Cover Slip | 60 mm × 22 mm, | 5 cover slips |
|---|---|---|
| | RNase-free | per pack |
| Incubation Chambers 2 | — | 2 |

The following components are included in the ProtoArray™ Mini-Biotinylation Module. Store at −20° C.

Sufficient reagents are included to perform 4 in vitro biotinylation reactions.

| Component | Composition | Amount | |
|---|---|---|---|
| Biotin-XX sulfosuccinimidyl | Lyophilized | 100 | μg |
| ester, sodium salt | | | |
| Sterile water | — | 1 | ml |
| Control Protein (BSA) | 2.5 μg/μl in PBS, pH 7.4 | 20 | μl |
| Biotinylation Gel Standard | 40 pmoles biotin conjugated | 20 | μl |
| (Biotinylated BSA) | per ml of BSA, in 1× | | |
| | Dilution Buffer | | |
| 1× Dilution Buffer | 1× PBS, pH 7.4 with 5 μg/ml | 1.7 | ml |
| | BSA | | |

The following components are included in the ProtoArray™ Biotinylation Purification Module. Store Purification Resin at 4° C. and Spin Columns and Collection Tubes at room temperature.

Sufficient reagents are included to perform 4 purifications.

| Component | Composition | Amount |
|---|---|---|
| Purification Resin | 50% slurry in 50 mM HEPES, | 3.6 ml |
| | pH 7.4 containing 0.1 M | |
| | NaCl and 1 mM sodium azide | |
| Spin Columns with Collection | — | 4 |
| Tubes | | |
| Collection Tubes (additional) | — | 4 |

The following components are included in the ProtoArray™ Biotinylation Assessment Module. Store at 4° C.

Sufficient reagents are included to perform 2 Western detections.

| Component | Composition | Amount |
|---|---|---|
| Western Blocking Buffer | Concentrated buffered saline | 8 ml |
| A | solution containing detergent | |
| Western Blocking Buffer | Concentrated Hammersten | 8 ml |
| B | casein solution | |
| Western Washing Buffer | Concentrated buffered saline | 20 ml |
| (16×) | solution containing detergent | |
| Chemiluminescent | Ready-to-use solution of CDP- | 5 ml |
| Substrate | Star ® chemiluminescent | |
| | substrate for alkaline | |
| | phosphatase | |
| Chemiluminescent | Nitro-Block-II ™ enhancer | 250 μl |
| Substrate Enhancer | | |
| Streptavidin-Alkaline | Supplied in 3 M NaCl, 1 mM | 25 μl |
| Phosphatase (AP) | MgCl₂, 0.1 mM ZnCl2, and 30 | |
| Conjugate | mM triethanolamine, pH 7.6 | |
| | The conjugate has 1600-2600 | |
| | units per ml of alkaline | |
| | phosphatase activity and a | |
| | concentration from 0.75-1.2 | |
| | mg/ml. Refer to the tube label | |
| | for the activity and | |
| | concentration for your lot of | |
| | conjugate. | |

The following components may optionally be included in the list.

| Product | Quantity | | Catalog no. |
|---|---|---|---|
| ProtoArray ™Mini-Biotinylation | 1 | kit | AL-01 |
| Kit | | | |
| NuPAGE ® Novex 4-12% Bis- | 1 | box | NP0321BOX |
| Tris Gel (1.0 mm, 10-well) | | | |
| SeeBlue ®Plus2 Pre-Stained | 500 | μl | LC5925 |
| Standard | | | |
| NuPAGE ® MOPS SDS Running | 500 | ml | NP0001 |
| Buffer (20×) | | | |
| NuPAGE ® MES SDS Running | 500 | ml | NP0002 |
| Buffer (20×) | | | |
| NuPAGE ®Transfer Buffer (20×) | 125 | ml | NP0006 |
| NuPAGE ®Antioxidant | 15 | ml | NP0005 |
| NuPAGE ® Sample Reducing | 250 | μl | NP0004 |
| Agent (10×) | | | |
| NuPAGE ® LDS Sample Buffer | 10 | ml | NP0007 |
| (4×) | | | |
| Nitrocellulose (0.45 μm) | 20 membrane/filter | | LC2001 |
| | paper sandwiches | | |
| XCell SureLock ™ Mini-Cell | 1 | unit | EI0001 |
| XCell II ™Blot Module | 1 | unit | EI9051 |
| ProQuest ™Two-Hybrid System | 5 | reactions | 10835-031 |
| with Gateway ® Technology | | | |
| Hybrid Hunter ™Yeast Two- | 1 | kit | K5000-01 |
| Hybrid System | | | |
| Phosphate Buffered Saline | 500 | ml | 10010-023 |
| (PBS), 1× | | | |

The major components of the Yeast ProtoArray™ PPI Kit include:
The Yeast ProtoArray™ PPI Proteome Microarray is a high-density protein microarray that allows you to screen your protein of interest (protein probe) against the Saccharomyces cerevisiae proteome
The Yeast ProtoArray™ PPI Control Microarray helps you to verify the biotinylation protocol and probing conditions
The ProtoArray™ Mini-Biotinylation Module is used for in vitro biotinylation of your protein probe
The ProtoArray™ Biotinylation Purification Module is used for removing free biotin from your biotinylated probe
The ProtoArray™ Biotinylation Assessment Module allows you to validate the level of biotinylation of your protein probe
The ProtoArray™ Buffers Modules include pre-made, qualified reagents for blocking and washing steps during probing

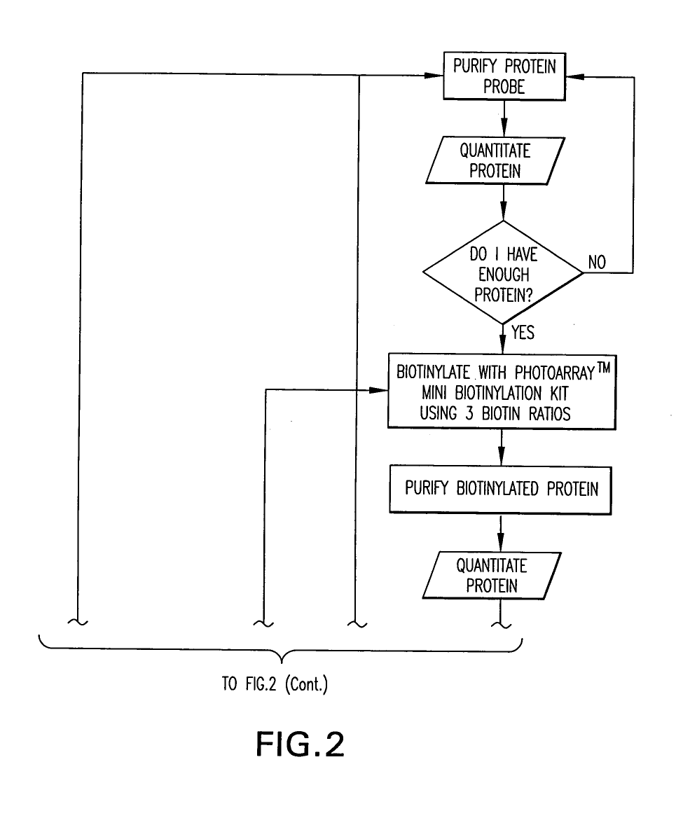

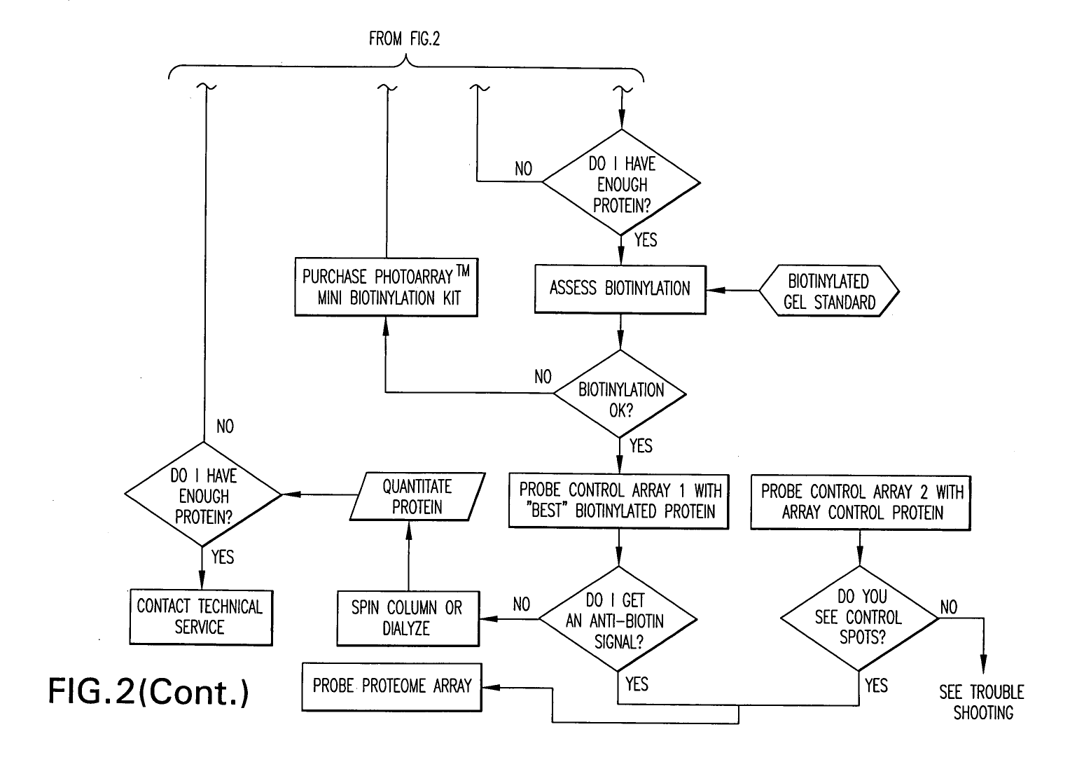

To use the Yeast ProtoArray™ PPI Kit, you will first in vitro iotinylate your protein of interest using Biotin-XX sulfosuccinimidyl ester, remove free biotin by gel filtration, and assess the efficiency and level of biotinylation using Western detection with Streptavidin-Alkaline Phosphatase (AP) Conjugate. Use the biotinylated protein to probe the Yeast ProtoArray™ PPI Control Microarray to verify protein biotinylation and probing conditions. Then probe the Yeast ProtoArray™ PPI Proteome Microarray with the biotinylated protein to detect protein-protein interactions. The ProtoArray™ detection protocol includes blocking the array, probing the array with your biotinylated protein, washing to minimize non-specific interactions, detecting interactions using Streptavidin-Alexa Fluor®647 conjugate, and scanning the array to view the results. For detailed experimental workflow, see FIG. 2.

Using the Yeast ProtoArray™ PPI Kit to detect protein-protein interactions offers the following advantages:
Provides a simple, rapid, and efficient method to identify protein interactions within a day
Includes qualified reagents for in vitro iotinylation, and buffers and detection reagents for probing, eliminating the need to prepare reagents.
Includes controls to verify biotinylation and Western detection protocols.
Allows screening of your protein of interest against >4000 yeast proteins
Suitable as a model to investigate interactions in higher eukaryotic systems.
Provides sensitive, stable, fluorescence detection using Alexa Fluor® 647 dye.
Built-in controls printed on each array to control for background and detection.
Arrays compatible with most commercially available fluorescent microarray scanners

The Yeast ProtoArray™ PPI Proteome Microarray is a high-density protein microarray containing proteins from the S. cerevisiae proteome. Each S. cerevisiae open reading frame (ORF) is expressed as a 5-GST-(Glutathione-S-Transferase)-6× His fusion protein, purified, and printed in duplicate on a nitrocellulose-coated glass slide. Using a labeled protein probe, you can screen against the S. cerevisiae proteome within a day to elucidate protein-protein interactions. Two proteome microarrays are included in each kit to allow you to assay protein interactions under different experimental conditions.

The Yeast ProtoArray™ PPI Control Microarray contains yeast protein interactors and various controls printed on a nitrocellulose-coated glass slide. The Control Microarrays are used to validate biotinylation and probing procedures prior to probing the yeast proteome microarray. Two arrays are included in the kit to allow you to test the biotinylation quality of the protein probe and protein interaction (probe with biotinylated calmodulin kinase). The use of nitrocellulose as a surface to print the arrays ensures maximum protein function since the nitrocellulose surface is known to be compatible with a variety of protein functions (Espejo et al., 2002; Kukar et al., 2002; Michaud et al., 2003).

To detect protein-protein interactions on the Yeast ProtoArray™, the protein probe must contain a label or tag to visualize the interaction of the probe with array proteins. The extremely high affinity of the biotin-streptavidin interaction makes biotin-protein conjugation an attractive method for probe labeling.

The ProtoArray™ Mini-Biotinylation Module provides a simple and efficient method to biotinylate small amounts of your protein probe using water-soluble Biotin-XX sulfosuccinimidyl ester. The module includes sufficient reagents to biotinylate your protein probe at 3 molar ratios. The biotinylated protein probe is detected using streptavidin conjugated to the fluorescent dye, Alexa Fluor®647 providing signal amplification and increased sensitivity.

After in vitro biotinylating the protein, the unconjugated or free biotin must be removed from the protein preparation as free biotin interferes with the probing procedure and increases the background on the array. The ProtoArray™ Biotinylation Purification Module provides spin columns and purification resin to rapidly remove free biotin by gel filtration.

Since each protein is different, the number of biotin molecules conjugated to the protein varies. To prevent under-biotinylation of the protein probe resulting in sub-optimal sensitivity or over-biotinylation of the protein probe resulting in loss of protein function, it is important to verify and assess the biotin conjugation reaction. The ProtoArray™ Biotinylation Assessment Module allows verification and assessment of the in vitro biotinylation reaction using Western detection. The module includes a Biotinylation Gel Standard, buffers, and detection reagents to perform Western transfer and chemiluminescent detection using Streptavidin-Alkaline Phosphatase conjugate. The band intensity of the protein probe is compared to the Biotinylation Gel Standard to verify biotinylation and assess the level of biotinylation. Based on the Western results, you can choose the protein biotinylated at a suitable level to probe the array and minimize erroneous results due to the use of over- or under-biotinylated probe.

The ProtoArray™ Buffers Modules include qualified reagents for blocking, washing, and detection steps required for probing Yeast ProtoArray™ Microarrays. The pre-made buffers provide consistent results and eliminate the time required to prepare reagents.

The module also includes HybriSlip™ cover slips that create a closed chamber when applied to the slide and hold a small reagent volume to minimize the amount of valuable probe used, and Incubation Chambers for washing the microarrays.

The high sensitivity, low background, signal stability, and commercial availability of fluorescence microarray scanners make fluorescence detection the preferred method for detecting protein-protein interactions on microarrays.

The Yeast ProtoArray™ PPI Kit includes Streptavidin-Alexa Fluor® 647 conjugate for detection. Alexa Fluor® 647 fluorophore is brighter and more stable than other commercially available dyes such as Cy™ Dyes and is more sensitive for detecting interactions on protein arrays. We have demonstrated that detection with Alexa Fluor® 647 produces at least 1.5-fold higher signal/background ratios than Cy5™ detection.

The ProtoArray™ Application Portal provides a web-based user interface to access ProtoArray™ specific information including various applications, resources, and online tools. The portal is also used to retrieve ProtoArray™ Lot Specific information which is required for analyzing the array data and identifying statistically significant interactions. Go to www.invitrogen.com/protoarray to visit the portal.

The Yeast ProtoArray™ PPI Proteome Microarray is a high-density protein microarray containing the majority of proteins from S. cerevisiae for protein interaction screening. Each S. cerevisiae open reading frame (ORF) is expressed as a 5-GST-6× His fusion protein, purified, and printed in duplicate on a nitrocellulose-coated glass slide. Two proteome microarrays are included in each kit to allow you to assay protein interactions under different experimental conditions.

The specifications for the Yeast ProtoArray™ Proteome Microarray are listed below.

| | |
|---|---|
| Dimensions: | 1 inch × 3 inch (25 mm × 75 mm) |
| Material: | Glass slide coated with nitrocellulose membrane |
| Membrane Size: | 20 mm × 60 mm |
| Membrane Properties: | Thickness: 15-20 μm; Pore Size: 0.2 μm |

Each microarray has a barcode for tracking samples. The barcode is also used to retrieve array specific information from the ProtoArray™ Application Portal.

The array specifications for the proteome microarray are listed below.

The proteins on the microarray are printed in 48 subarrays and are equally spaced in vertical and horizontal directions.

| Total Subarrays: | 48 (4 columns × 12 rows) |
|---|---|
| Subarray Size: | 4000 μm × 4000 μm |
| Subarray Dimensions: | 8 rows × 8 columns |
| Median Spot Diameter: | ˜150 μm |
| Spot Center to Center Spacing: | 500 μm |
| Distance Between Subarrays: | 500 μm |
| Total Spots: | 3072 |
| Replicates per Sample: | 2 |
| Control and Blank Spots | 2056 |

The yeast proteome collection is derived from the S. cerevisiae clone collection of 5800 yeast ORFs (Zhu et al., 2001). Each S. cerevisiae open reading frame (ORF) is expressed as a 5′-GST-6× His fusion protein in the yeast expression vector pEG-KG (Mitchell et al., 1993). The identity of each clone was verified using 5′-end sequencing and the expression of GST-tagged fusion protein by each clone was confirmed with Western immunodetection using an anti-GST antibody. Once the identity of each clone was confirmed, the proteins from each clone were expressed and purified using high-throughput procedures.

Briefly yeast stocks were grown in growth media, protein expression was induced with galactose, and cell lysates prepared. The proteins were purified using glutathione affinity chromatography, eluted, and purified proteins were used for spotting the proteome microarray.

The purified yeast proteins are printed on nitrocellulose-coated slides in a dust-free and temperature and humidity controlled environment to maintain consistent quality of microarrays. The arrays are printed using an automated process on an arrayer that is extensively calibrated and tested for printing Yeast ProtoArray™ PPI Microarrays.

The Yeast ProtoArray™ PPI Proteome Microarrays are ideal for detecting reciprocal interactions since the microarrays are manufactured under highly controlled conditions to ensure maximum protein function.

Once you have identified a positive interaction using the Yeast ProtoArray™ PPI Proteome Microarray, use the identified interacting protein from the array as a probe for probing another proteome microarray.

The Yeast ProtoArray™ PPI Proteome Microarrays are produced using rigorous production and quality control procedures with an integrated data management system to ensure consistent results with every array and maximum inter-and intra-lot reproducibility.

Pre-Printing Quality Control

Prior to production, the arrayer and supporting components are tested and adjusted to production specifications. The quality and performance of pins is critical and all pins are extensively tested and calibrated. To maintain protein stability and function, arrays are printed at 6° C. under controlled environmental conditions.

Post-Printing Quality Control

After production each microarray is visually inspected for obvious defects that could interfere with the experimental results. To control for the quality of the printing process, several microarrays from each lot are probed with an anti-GST antibody. Since the proteins contain a GST fusion tag, probing the microarrays with an anti-GST antibody allows identification of irregular spot morphology or missing spots. The arrays are functionally qualified by probing control proteins to detect the appropriate protein-protein interactions.

The layout of a Yeast ProtoArray™ PPI Proteome subarray (16×16) is shown below. Each yeast protein is printed in duplicate.

For details on the array proteins, go the ProtoArray™ Application Portal at www.invitrogen.com/protoarray to download lot specific array information.

Y=Yeast proteins and C=Control Proteins (see below for details).

| | 1 | 2 | 3 | 4 | 5 | 6 | 7 | 8 | 9 | 10 | 11 | 12 | 13 | 14 | 15 | 16 |
|---|---|---|---|---|---|---|---|---|---|---|---|---|---|---|---|---|
| 1 | c | c | c | c | c | c | c | c | c | c | c | c | c | c | c | c |
| 2 | Y | Y | Y | Y | Y | Y | Y | Y | Y | Y | Y | Y | Y | Y | Y | Y |
| 3 | Y | Y | Y | Y | Y | Y | Y | Y | Y | Y | Y | Y | Y | Y | Y | Y |
| 4 | Y | Y | Y | Y | Y | Y | Y | Y | Y | Y | Y | Y | Y | Y | Y | Y |
| 5 | Y | Y | Y | Y | Y | Y | Y | Y | Y | Y | Y | Y | Y | Y | Y | Y |
| 6 | Y | Y | Y | Y | Y | Y | Y | Y | Y | Y | Y | Y | Y | Y | Y | Y |
| 7 | Y | Y | Y | Y | Y | Y | Y | Y | Y | Y | Y | Y | Y | Y | Y | Y |
| 8 | c | c | c | c | c | c | c | c | c | c | c | c | c | c | c | c |
| 9 | Y | Y | Y | Y | Y | Y | Y | Y | Y | Y | Y | Y | Y | Y | Y | Y |
| 10 | Y | Y | Y | Y | Y | Y | Y | Y | Y | Y | Y | Y | Y | Y | Y | Y |
| 11 | Y | Y | Y | Y | Y | Y | Y | Y | Y | Y | Y | Y | Y | Y | Y | Y |
| 12 | Y | Y | Y | Y | Y | Y | Y | Y | Y | Y | Y | Y | Y | Y | Y | Y |
| 13 | Y | Y | Y | Y | Y | Y | Y | Y | Y | Y | Y | Y | Y | Y | Y | Y |
| 14 | Y | Y | Y | Y | Y | Y | Y | Y | Y | Y | Y | Y | Y | Y | Y | Y |
| 15 | c | c | c | c | c | c | c | c | c | c | c | c | c | c | c | c |
| 16 | c | c | c | c | c | c | c | c | c | c | c | c | c | c | c | c |

The Yeast ProtoArray™ Control Microarray contains yeast protein interactors and various controls printed on a nitrocellulose-coated glass slide. The Control Microarrays allow you to validate biotinylation and probing procedures prior to probing the Yeast PhotoArray™ PPI Proteome Microarray.

Details on the Yeast ProtoArray™ PPI Control microarray are described in this section.

The specifications for the Yeast ProtoArray™ PPI Control Microarray are listed below.

| | |
|---|---|
| Dimensions: | 1 inch × 3 inch (25 mm × 75 mm) |
| Material: | Glass slide coated with nitrocellulose membrane |
| Membrane Size: | 20 mm × 60 mm |
| Membrane Properties: | Thickness: 15-20 μm; Pore Size: 0.2 μm |

Each microarray has a barcode for tracking samples. The barcode is also used to retrieve array specific information from the portal.

The control array specifications are listed below.

The proteins on the microarray are printed in 48 subarrays and are equally spaced in vertical and horizontal directions.

| Total Subarrays: | 48 (4 columns × 12 rows) |
|---|---|
| Subarray Size: | 4000 μm × 4000 μm |
| Subarray Dimensions: | 8 rows × 8 columns |
| Median Spot Diameter: | ˜150 μm |
| Spot Center to Center Spacing: | 500 μm |
| Distance Between Subarrays: | 500 μm |
| Total Spots: | 3072 |
| Replicates per Sample: | 2 |

Various other proteins and controls are printed on each Yeast ProtoArray™ PPI Proteome and Control Microarray to verify background, labeling, and detection. Note: The location of controls printed on the Yeast ProtoArray™ PPI Proteome and Control microarray are different.

The table below lists the controls printed on each Yeast ProtoArray™ PPI microarray.

| Protein | Function |
|---|---|
| Buffer only | Detects non-specific interaction with buffer |
| BSA | Serves as negative control for protein |
| | interactions |
| Alexa Fluor ®Antibody | For orientation of the microarray |
| Anti-biotin Antibody | Detects biotin labeled probe |
| Biotinylated Antibody | Detects Alexa Fluor ® 647 conjugated |
| | streptavidin |
| GST Protein Gradient | Detects non-specific binding to GST |
| Control Interactors | Serves as a positive control for interactions |
| | using biotinylated calmodulin or calmodulin |
| | kinase probes |

The Yeast ProtoArray™ PPI Control Microarrays are produced using the same rigorous production and quality control procedures used to produce the yeast proteome microarrays. In addition, the control arrays are functionally qualified by probing the arrays with a biotinylated yeast calmodulin kinase probe to detect the appropriate interaction with calmodulin

A detailed workflow is shown in FIG. 2.

For details on the subarray layout and control protein spots, go the Layout ProtoArray™ Application Portal at www.invitrogen.com/protoarray.
Resuspend the purified protein probe in a buffer (≦50 mM) that does not contain any primary amines such as ammonium ions, Tris, glutathione, imidazole, or glycine. If the buffer contains primary amines, sufficiently dialyze proteins against 50 mM HEPES buffer, pH 7.4 containing 100 mM NaCl or PBS.
You will need to know the approximate molecular weight of your protein and the protein must be >15 kDa.
For proteins purified using metal chelating column chromatography (ProBond™ resin or Ni—NTA resin), perform dialysis against 2 changes of PBS to significantly lower the imidazole concentration.
If you are using a recombinant protein probe, you may check the functionality of the protein using a method of choice.
Low concentrations (<0.1%) of sodium azide or thimerosal in the protein solution have no effect on the biotinylation reaction.

You will need at least 150 μg protein of purified protein probe at a protein concentration of 2.5 mg/ml.

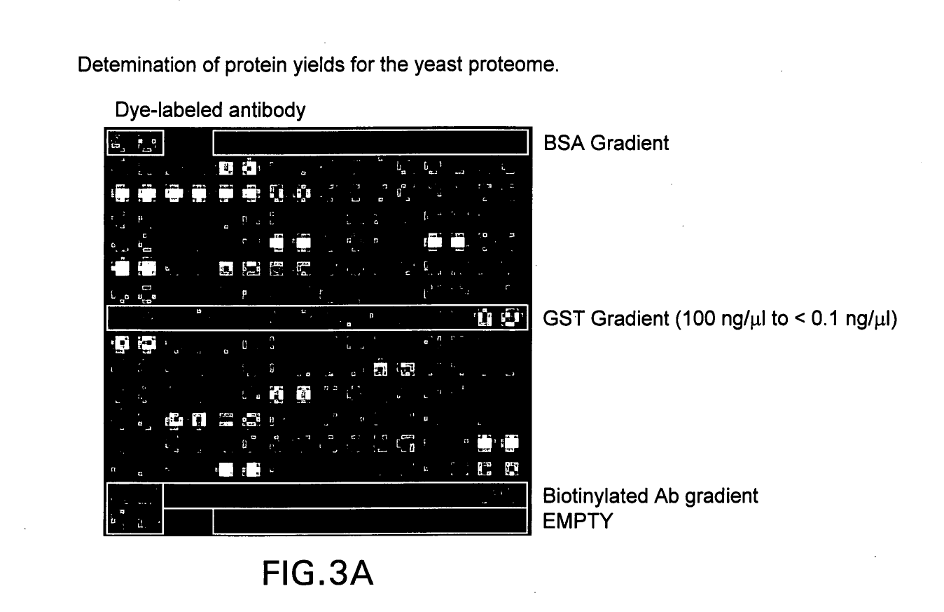

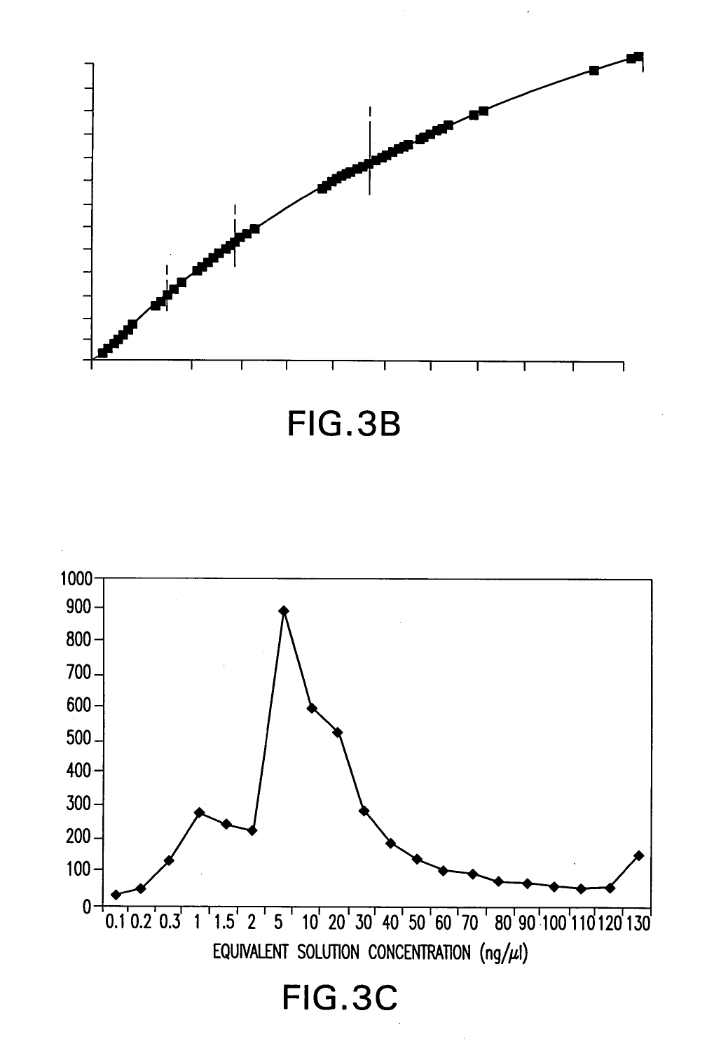

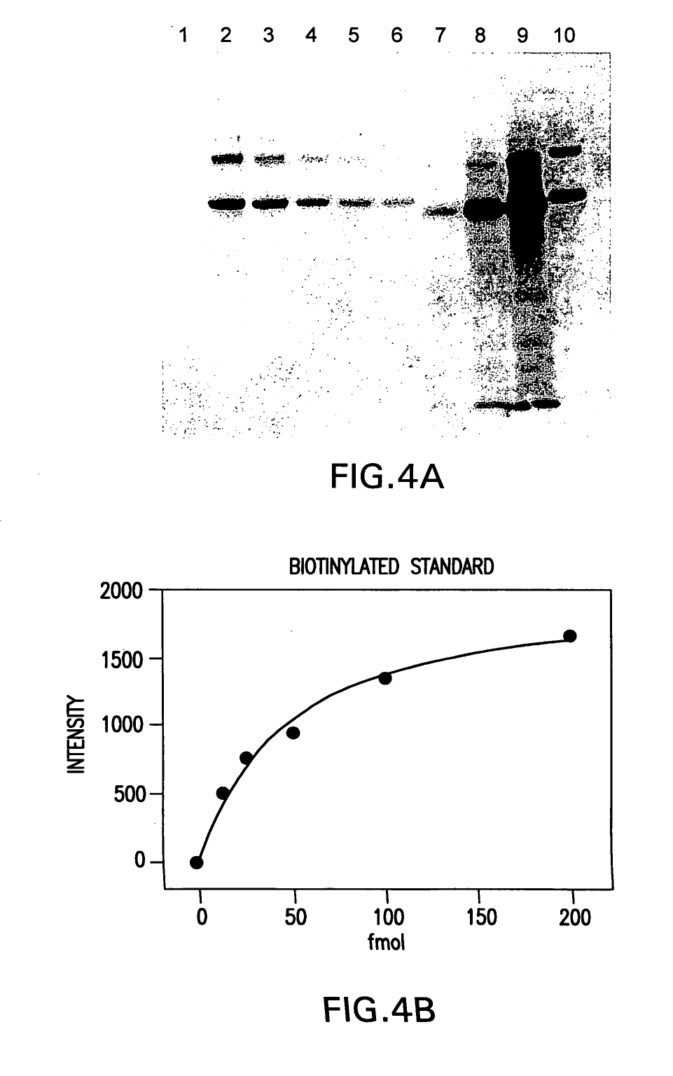

In vitro Biotinylation can be performed by an method known to the skilled artisan. An illustrative determination of protein yields for the yeast proteome is shown in FIG. 3. An illustrative assessment of biotinylation of yeast calmodulin kinase using Western blot analysis is shown in FIG. 4.

Due to the differences in protein (e.g., lysine residues) the level of biotinylation on each protein will vary. To obtain the best results with the biotinylated protein probe for use with the Yeast ProtoArray™, it is important to determine and assess the biotinylation for your protein sample.

Instructions for assessing protein biotinylation with Western blotting and chemiluminescent detection using the ProtoArray™ Biotinylation Assessment Module are described in this section.

The ProtoArray™ Biotinylation Assessment Module provides an efficient and sensitive method of assessing the level of biotinylation and includes a Biotinylation Gel Standard.

Assessment is performed by SDS-PAGE of biotinylated protein samples and the Biotinylated Gel Standard, Western transfer to nitrocellulose membranes (see Note below), detection with Streptavidin-AP conjugate, and visualization using a chemiluminescent substrate. The band intensities of the biotinylated protein samples are compared to the Biotinylation Gel Standard to assess the level of biotinylation.
1. Perform SDS-PAGE using biotinylated protein samples and the Biotinylation Outline Gel Standard from the kit.
2. Transfer proteins to nitrocellulose membrane.
3. Perform Western chemiluminescent detection with Streptavidin-AP conjugate.
4. Verify and assess the level of biotinylation for your protein probe.

We recommend using nitrocellulose membranes for Western detection to assess protein biotinylation. Our results with ProtoArray™ Biotinylation Assessment Module have demonstrated lower sensitivity and higher background using PVDF membranes for Western detection.

A large variety of pre-cast gels for SDS-PAGE are available from Invitrogen. We recommend using NuPAGE® Novex Bis-Tris Gels and instructions are provided below to prepare samples for SDS-PAGE with NuPAGE® Gels. If you are using Tris-Glycine or other gels, refer to the manufacturer's recommendations for sample preparation.

You will need the following items: ProtoArray™ Biotinylation Assessment Module (supplied with the ProtoArray™ kit); 1× Dilution Buffer and Biotinylation Gel Standard (included in the ProtoArray™ Mini-Biotinylation Module); Aliquot of purified biotinylated protein probe and BSA 2 NuPAGE® Novex Bis-Tris Gels NuPAGE®; Sample Reducing Agent NuPAGE®; LDS Sample Buffer NuPAGE®; MES or MOPS SDS Running Buffer; Nitrocellulose membranes; Electrophoresis and blotting apparatus; Deionized water; Heating block set at 70° C.; Appropriate staining container for Western blotting; Molecular weight markers

Prepare the following dilutions of the Biotinylation Gel Standard to generate a standard curve for SDS-PAGE.

Each ml of the Biotinylation Gel Standard contains 40 pmoles of biotin conjugated to BSA and is used for assessing biotinylation.
1. Thaw the Biotinylation Gel Standard.
2. Prepare 2-fold serial dilutions to obtain 20 fmoles/μl, 10 fmoles/μl, 5 fmoles/μl, 2.5 fmoles/μl and 1.25 fmoles/μl. For each dilution, dilute the standard with 1× Dilution Buffer to a final volume of 20 μl.

3. Use each dilution of the standard to prepare samples for SDS-PAGE as follows: Sample 20 μl 1× Dilution Buffer 6 μl NuPAGE® LDS Sample Buffer (4×) 10 μl

| | |
|---|---|
| Sample | 20 μl |
| 1× Dilution Buffer | 6 μl |
| NuPAGE ® LDS Sample Buffer (4×) | 10 μl |
| NuPAGE ® Reducing Agent (10×) | 4 μl |
| Total Volume | 40 μl |

4. Heat the samples at 70° C. for 10 minutes.
5. Load 20 μl sample on a NuPAGE® Novex 4-12% Bis-Tris Gel as described

Using these samples will generate a standard curve containing 200 fmoles, 100 fmoles, 50 fmoles, 25 fmoles, and 12.5 fmoles of the Biotinylated Gel Standard.

A formula is included below for your convenience to generate a stock solution for each of your protein samples after column purification. If you are an experienced user and are familiar with protein molar calculations, you may use your own method for calculation.

Use the formula below to calculate the final volume of the sample to generate a 200 fmoles/μl stock solution from 1 μl of column purified material for each of the 3 protein biotinylation reactions (treated at 27:1, 9:1, and 3:1 molar ratio) and control BSA biotinylation reaction (treated at 9:1 molar ratio).

You will need to know the protein concentration in mg/ml and the approximate molecular weight for each protein sample. The molecular weight of BSA (used for control biotinylation reaction) is 66,700 Da.

$$\begin{matrix}\underline{5 ⨯ {10}^{6} ⨯ protein concentration ( mg \text{/} ml )} = final volume in µl \\ M W ( D a )\end{matrix}$$

MW is the molecular weight of the protein in Daltons.

### EXAMPLE

If the protein concentration of your sample after column purification is 0.5 mg/ml and the MW of your protein is 50,000 Da, calculate the final volume as follows:

$$\begin{matrix}\underline{5 ⨯ {10}^{6} ⨯ 0.5} = 50 µl \\ 50000\end{matrix}$$

Dilute 1 μl of each sample for this example with 49 μl 1× Dilution Buffer to generate a 200 fmoles/μl stock solution for each sample.

Prepare the following dilutions of the biotinylated protein sample and BSA Control Protein after column purification for assessing biotinylation.
1. Prepare a 200 fmoles/μl stock solution for each sample using the formula for SDS-PAGE described above.
2. From the 200 fmoles/μl stock solution for each sample, prepare the following dilutions:
   Dilute 1 μl of 200 fmoles/μl solution from each sample with 9 [tl with 1× Dilution Buffer to generate the 20 fmoles/μl sample (total volume is 10 μl).
   Dilute 2 μl of 20 fmoles/μl solution from each sample with 6 μl with 1× Dilution Buffer to generate the 5 fmoles/μl sample (total volume is 8 μl).

3. Prepare 8 samples for SDS-PAGE using 5 μl of each dilution above as follows:

| | |
|---|---|
| Sample | 5 μl |
| 1× Dilution Buffer | 8 μl |
| NuPAGE ® LDS Sample Buffer (4×) | 5 μl |
| NuPAGE ® Reducing Agent (10×) | 2 μl |
| Total Volume | 20 μl |

4. Heat the samples at 70° C. for 10 minutes.
5. Load 20 μl sample on a NuPAGE® Novex 4-12% Bis-Tris Gel as described on the below. The final amount for each sample is listed on the below.

After preparing samples, perform SDS-PAGE. You will need 2 NuPAGE® Novex Bis-Tris mini-gels for analysis. The recommended loading pattern and final amount for each sample is listed below. Load 20 μl of each sample on the gel and 10 μl of a molecular weight protein standard.

For NuPAGE® Novex Bis-Tris Gels, perform SDS-PAGE at 200 V for 35-50 minutes using NuPAGE® MES or MOPS Running Buffer with an XCell SureLock™ Mini-Cell.

After electrophoresis is complete, proceed to blotting, below.

Transfer proteins from the two gels to nitrocellulose membranes using a suitable transfer apparatus. Note: PVDF membranes are not recommended for use in Western transfer when using this protocol.

For NuPAGE® Novex Bis-Tris Gels, perform transfer at 30 V for 1 hour using 1× NuPAGE® Transfer Buffer with 10% methanol.

After transfer, proceed to detection and visualization as described below.

To obtain the best detection results with reagents included in the ProtoArray™ Biotinylation Assessment Module, follow these guidelines:
Use a single, clean container for each blot.
Avoid touching the working surface of the membrane, even with gloves.
Avoid cross-contamination of system solutions especially with the alkaline phosphatase substrate solution.
Perform all washing, blocking, and incubation steps on a rotary shaker rotating at 1 revolution/second.
Add solutions to the trays slowly, at the membrane edge, to avoid bubbles forming under the membrane. Decant from the same corner of the dish to ensure complete removal of previous solutions.

Prepare the solutions for analyzing 2 nitrocellulose membranes using the reagents included in the kit as described below.

| Solution | For Nitrocellulose Membrane | | |
|---|---|---|---|
| Blocking Solution | Ultra filtered Water | 28 | m |
| | Western Blocking Solution A | 8 | ml |
| | Western Blocking Solution B | 4 | ml |
| | Total Volume | 40 | ml |
| Streptavidin-AP | Streptavidin- AP Conjugate | 5 | μl to |
| Solution (1:4000) | Blocking Solution (above) | 20 | ml |
| Antibody Wash | Ultra filtered Water | 150 | ml |
| | Antibody Wash Solution (16×) | 10 | ml |
| | Total Volume | 160 | ml |
| Chemiluminescent | Chemiluminescent Substrate | 4.75 | ml |
| Substrate | Chemiluminescent Substrate | 0.25 | ml |
| | Enhancer | | |
| | Total Volume | 5 | ml |

1. lace each membrane in 10 ml of the Blocking Solution in a staining container. Incubate for 30 minutes on a rotary shaker set at 1 revolution/sec. Decant the Blocking Solution.
2. Rinse the membrane with 20 ml of water for 5 minutes, then decant. Repeat once.
3. Incubate the membrane in 10 ml of Streptavidin-AP Solution (1:4000) for 30 minutes, then decant.
4. Wash the membrane for 5 minutes with 20 ml of Antibody Wash, then decant. Repeat 3 times.
5. Rinse the membrane with 20 ml of water for 2 minutes, then decant. Repeat twice.
6. Place the membrane on a sheet of transparency plastic. Do not allow the membrane to dry out.
7. With a clean pipette, evenly apply 2.5 ml of the Chemiluminescent Substrate to the membrane surface without touching the membrane surface. Let the reaction develop for 5 minutes.
8. Blot the excess Chemiluminescent Substrate solution from the membrane surface with the filter paper. Do not allow the membrane to dry out.
9. Cover the membrane with another clean piece of transparency plastic to prepare a membrane sandwich for luminography. Expose an X-ray film (we recommend Kodak X-OMAT AR films) to the membrane sandwich for 3-6 seconds (see below for an example of the blot).
   The alkaline phosphatase-activated CDP-Star® produces a maximum light emission wavelength at 466 nm to 461 nm, depending on the membrane environment of the reaction.
10. Proceed to assessing the Western detection results as described below.
1. Verify that the protein sample and Control Protein (BSA) is biotinylated. You can also perform a densitometry scan. See below for an example of a Western blot.
2. Compare the band intensities of 3 different molar ratios of biotinylated protein samples from Step 9, above to the BSA Control Protein and Biotinylation Gel Standard on the blot.
3. Use the biotinylated protein sample that gives the best signal at the lowest biotinylation molar ratio to probe the control array and proteome array.
   The below shows results of a biotinylation experiment and provides guidelines on interpreting your biotinylation results.

For troubleshooting biotinylation problems.

To interpret the results, compare the band intensities of your biotinylated protein to the BSA Control Protein and Biotinylation Gel Standard as described below to select a properly biotinylated protein probe (˜3-5 biotin molecules/protein).

The BSA Control Protein (25 fmoles, lane 10, above) is modified with 3-5 biotin molecules per polypeptide. Loading 25 fmoles BSA Control Protein is equivalent to loading 75-125 fmoles biotin. The band intensity of 25 fmoles BSA Control Protein is approximately similar to the band intensity of 100 fmoles Biotinylation Gel Standard (lane 3, above).

For a protein with average lysine content (˜8%), biotinylating at a molar ratio of 9:1 usually incorporates 3-5 biotin molecules/protein. The band intensity of 25 fmoles of protein probe biotinylated at 9:1 (lane 8, above) should be approximately similar to the band intensity of BSA Control Protein (lane 10, above) or 100 fmoles Biotinylation Gel Standard (lane 3, above). Based on the biotinylation results of the example shown in the gel, you can use calmodulin kinase biotinylated at 9:1 molar ratio for probing experiments.

The Yeast ProtoArray™ PPI Control Microarrays allow you to verify in vitro biotinylation labeling and probing conditions. Probe the Control Arrays prior to probing the proteome arrays.

Instructions are described in this section to probe Yeast ProtoArray™ PPI Control Microarrays.

The ProtoArray™ Buffers Module A and B supplied with the Yeast ProtoArray™ PPI Kit include qualified reagents for blocking, washing, and detection steps required for probing Yeast ProtoArray™ Microarrays. The pre-made buffers provide consistent results and eliminate the time required to prepare reagents.

The module also includes HybriSlip™ cover slips that create a closed chamber when applied to the slide and hold a small reagent volume to minimize the amount of valuable probe used, and Incubation Chambers for washing the microarrays.

You will need the following items:
2 Yeast ProtoArray™ PPI Control Microarrays (included in the kit)
ProtoArray™ Buffers Module A (included with the kit)
ProtoArray™ Buffers Module B (included with the kit)
Streptavidin-Alexa Fluor® 647 Conjugate (keep on ice in dark until immediately before use)
Biotinylated Protein Probe in Probing Buffer (see below)
Array Control Protein in Probing Buffer (see below)
Sterile 50 ml conical tube (2)
Ice bucket
Microarray slide holder
Centrifuge equipped with a plate holder
Deionized water

The Yeast ProtoArray™ PPI Control Microarray can only be used once. Do not re-use the microarrays or reprobe the same microarray with another probe.

Experimental Outline
1. Block Yeast ProtoArray™ PPI Control Microarrays.
2. Probe with biotinylated protein probe and calmodulin kinase.
3. Perform secondary probing with Streptavidin-Alexa Fluor® 647.
4. Dry the arrays for scanning.

Since proteins are sensitive to various environmental factors, each array is produced in an environment-controlled facility to ensure protein integrity and maintain consistency.

To obtain the best results and avoid any damage to the array or array proteins, always handle the Yeast ProtoArray™ Microarrays with care using the following guidelines:
Always wear clean gloves and while handling microarrays
Do not touch the surface of the array to avoid any damage to the array surface resulting in uneven or high background
Maintain the array and reagents at 4° C. during the experiment
To prevent condensation on the array that may reduce protein activity or alter spot morphology, remove array from the mailer and immerse the array immediately in blocking solution when performing an experiment
Perform array experiments at a clean location to avoid dust or contamination and filter solutions if needed (particles invisible to eyes can produce high background signals and cause irregular spot morphology)
Avoid drying of the array and ensure the array is completely covered with the appropriate reagent during the probing procedure
Always dry the array prior to scanning and scan the array on the same day at the end of the experiment
Avoid drying the arrays using compressed air or commercial aerosol sprays
Avoid exposing the array to light after probing with Streptavidin-Alexa Fluor® 647 conjugate

Use the following Biotinylated proteins to probe the Yeast ProtoArray™ PPI Control Microarrays.

Biotinylated Protein: Reacts with anti-biotin antibody printed on the Control Array and is used to indicate poor biotinylation or the presence of free biotin. Use the biotinylated protein sample that gives the best signal on a Western blot at the lowest biotinylation molar ratio to probe the control array.

Biotinylated Calmodulin Kinase (included in the kit): Reacts with calmodulin printed on the Control Arrays and is used to verify probing procedure and reagents.

Prepare the following buffers fresh prior to use. The recipes below provide sufficient buffers to probe 2 microarrays.

PBST Blocking Buffer

1× PBS

1% BSA

0.1% Tween 20

1. Use reagents provided in the kit to prepare 50 ml PBST Blocking Buffer as follows:

| | | |
|---|---|---|
| Blocking Buffer (10×) | 5 | ml |
| 30% BSA | 1.7 | ml |
| Deionized water | to 50 | ml |

2. Mix well (do not vortex) and store on ice until use.

Probing Buffer

1× PBS

5 mM MgCl₂

0.5 mM DTT

0.05% Triton X-100

5% Glycerol

1% BSA

1. Use reagents provided in the kit to prepare 300 ml Probing Buffer as follows:

| | | |
|---|---|---|
| Probe Buffer (5×) | 60 | ml |
| 1 M DTT | 150 | μl |
| 1 M MgCl₂ | 1.5 | ml |
| 30% BSA | 10 | ml |
| Deionized water | to 300 | ml |

2. Mix well and store on ice until use.

After preparing buffers, immediately return the remaining Probe Buffer, Blocking Buffer, and MgCl₂ to 4° C., and BSA and DTT to −20° C.

Array Control Protein (Calmodulin Kinase)

Mix 12 μl Array Control Protein included in the kit with 120 μl Probing Buffer. Mix well (do not vortex) and store on ice until use.

Biotinylated Protein Probe

You will need 120 μl of probe. Use the biotinylated protein sample that gives the best signal on Western blot at the lowest biotinylation molar ratio and dilute the probe to 50 μg/ml in Probing Buffer. Mix well (do not vortex) and store on ice until use.

Before starting the probing procedure, make sure you have all items on hand especially buffers (above), biotinylated probes in Probing Buffer (above), Incubation Chambers (included in the kit), and HybriSlips™ (included in the kit).

Make sure the buffers are cold. Store buffers on ice until use. Place the Incubation Chambers on ice to chill the chamber until use.
1. Remove Yeast ProtoArray™ PPI Control Microarrays from box.
2. Perform blocking in the Incubation Chamber or mailer as described below:
   Incubation Chamber: Remove 2 Yeast ProtoArray™ PPI Control Microarrays from the mailer and insert microarrays into the rails of the Incubation Chamber kept on ice. Add 30 ml PBST Blocking Buffer to the chamber containing 2 arrays. Incubate for 1 hour in the cold room with gentle shaking (˜50 rpm). Ensure that the 2 microarrays are placed properly in the chamber with the printed (white) side facing up.
   Mailer: You can also add 30 ml PBST Blocking Buffer to the mailer containing 2 Yeast ProtoArray™ PPI Control Microarrays and perform the blocking step in the mailer as above.
3. Decant the PBST Blocking Buffer. Drain excess buffer by inverting the Incubation Chamber or mailer on paper towels for a few seconds. Remove arrays from the chamber or mailer. Tap one edge of arrays gently on a laboratory wipe for a few seconds to drain any buffer without allowing the array to dry. Place arrays on a clean, flat surface with the printed side of the array facing up.
4. Pipette 120 μl of biotinylated protein probe (50 μg/ml) prepared in Probing Buffer on top of 1 array without touching the array surface. Add 120 μl biotinylated calmodulin kinase (50 μg/ml) in Probing Buffer on top of the second Control Array. The liquid will quickly spread over the nitrocellulose membrane.
5. Carefully lift the HybriSlip™ cover slip from the support liner with forceps and lay the clear side of HybriSlip™ cover slip on the array without trapping any air-bubbles. The HybriSlip™ is designed to exactly cover the membrane area. Gently adjust the HybriSlip™ to remove any air-bubbles.
6. Insert each assembly (array with Hybrislip™) into a separate 50 ml conical tube with the printed side of the array facing up. Cap the conical tube.
7. Place the conical tube on a flat surface such that the printed side of the array is facing up and the tube is as level as possible. If needed, you can tape the conical tube on the flat surface to avoid any accidental disturbances. Incubate the array in the tube for 1.5 h at 4° C. without shaking.
8. Remove the array from each conical tube and insert arrays diagonally (see Note, below) into the Incubation Chamber kept on ice.
   Note: The microarray with HybriSlip™ will not fit on the rails of the chamber.

You need to insert the microarray diagonally into the chamber.
9. Using a sterile pipette, add 30 ml of Probing Buffer to the chamber wall while keeping the chamber on ice. Avoid pipetting buffer directly onto the surface of the array. The addition of buffer separates the HybriSlip™ from the array. Carefully remove the HybriSlip™ with forceps without touching the array surface with forceps. Discard the HybriSlip™. The array can now be repositioned on the chamber rails, if desired.
10. Incubate the array in Probing Buffer for ˜1 minute on ice. Decant the Probing Buffer. Invert chamber on paper towels for a few seconds to drain excess buffer.
11. Add 30 ml Probing Buffer to the chamber and incubate arrays in Probing Buffer for ˜1 minute on ice.
12. Prepare Streptavidin-Alexa Fluor® 647 solution by mixing 6 μl Streptavidin-Alexa Fluor® 647 conjugate (included with the kit) with 30 ml Probing Buffer.
13. After the 1 minute incubation with Probing Buffer, decant the buffer. Invert chamber on paper towels for a few seconds to drain excess buffer. Add 30 ml of Streptavidin-Alexa Fluor® 647 solution from Step 12 to the chamber.
14. Incubate the chamber for 30 minutes on ice in dark (cover the ice bucket). Decant the buffer. Invert the chamber on paper towels for a few seconds to drain excess buffer.
15. Slowly add 30 ml Probing Buffer onto the chamber wall while keeping the chamber on ice. Avoid pipetting buffer directly onto the surface of the array.
16. Incubate array in Probing Buffer for ˜1 minute on ice. Decant the buffer. Drain excess buffer by inverting chamber on paper towels for a few seconds.
17. Repeat Steps 15-16 two more times, using 30 ml Probing Buffer each time.
18. Proceed to Drying the Arrays, below.

Drying the Arrays
1. Remove arrays from the chamber. Tap one edge of arrays gently on a laboratory wipe for a few seconds to drain any buffer.
2. Place arrays in a slide holder in a vertical orientation. Ensure the array is properly placed and is secure in the holder to prevent any damage to the array during centrifugation.
3. Centrifuge the arrays in the slide holder at 800×g for 3-5 minutes in a centrifuge equipped with a plate rotor at room temperature.
4. Place arrays in a slide box and keep the box with the lid open in dark for 30 minutes at room temperature for drying the arrays.
5. Scan the arrays using a fluorescent microarray scanner after the arrays are completely dry with no translucent areas.
   6. After confirming the appropriate interactions with Control Arrays and verifying that the biotinylated protein produces the proper signal with anti-biotin spots and gives acceptable background, proceed to probing the Yeast ProtoArray™ PPI Proteome Microarray, below.

At the end of probing experiments, clean the Incubation Chambers properly and rinse with sterile water before re-using the chambers.

After verifying the quality of the in vitro biotinylated protein and probing conditions, probe the Yeast ProtoArray™ PPI Proteome microarray using your biotinylated protein as described below.

You will need the following items:
Yeast ProtoArray™ PPI Proteome Microarrays (included in the kit)
ProtoArray™ Buffers Module A
ProtoArray™ Buffers Module B
Biotinylated Probe in Probing buffer (below)
Streptavidin-Alexa Fluor® 647 conjugate (keep on ice in dark until immediately before use)
Sterile 50 ml conical tube
Microarray slide holder
Centrifuge equipped with a plate holder
Deionized water

The Yeast ProtoArray™ PPI Proteome Microarray can only be used once. Do not re-use the arrays or reprobe the same array with another probe.

Experimental Outline
1. Block Yeast ProtoArray™ PPI Proteome Microarrays.
2. Probe with biotinylated protein probe.
3. Detect with Streptavidin-Alexa Fluor® 647 conjugate.
4. Dry the array for scanning.

You can probe the proteome arrays using a different probe concentration or biotinylation molar ratio. The recommended biotinylated protein probe concentration range for probing proteome arrays is 5-50 μg/ml.

Probing Options
You can probe both arrays simultaneously using one proteome array as a negative control (an image of the negative control for Yeast ProtoArray™ PPI Proteome Microarray is available on the ProtoArray™ Application Portal).

OR

You can probe one array with an initial probe concentration or molar ratio. If the initial signal is strong with low background, confirm the initial results with the second array using the same experimental conditions. If the initial results indicate weak signal and unacceptable signal-to-noise ratio, probe the second array with a different probe concentration or molar ratio as described in the table below:

| Probe first array . . . | And . . . | Then Probe Second Array . . . |
|---|---|---|
| With 5 μg/ml probe | Weak signal | With 50 μg/ml probe |
| With 50 μg/ml probe | High background | With 5 μg/ml probe |
| With 9:1 molar ratio | Weak signal | With 27:1 molar ratio |
| With 27:1 molar ratio | High background | With 3:1 or 9:1 molar ratio |

Prepare BST Blocking Buffer and Probing Buffer as described above.

You will need 120 μl of the biotinylated probe. Use the biotinylated probe that gives the best signal on the Western blot at the lowest biotinylation molar ratio. Dilute the probe to 5-50 μg/ml in Probing Buffer. Mix well (do not vortex) and store on ice until use.

Before starting the probing procedure, make sure you have all items on hand especially buffers (above), biotinylated probes in Probing Buffer (above), Incubation Chambers (included in the kit), and HybriSlips™ (included in the kit).

Make sure the buffers are cold. Store buffers on ice until use. Place the Incubation Chamber on ice to chill the chamber until use.

The options for probing the array with different probe concentration or molar ratios are described on the above.
1. Probe the Yeast ProtoArray™ PPI Proteome Microarray using the procedure described
2. Dry the array as described
3. Scan and analyze results as described on the below.

Scanning Arrays

The arrays can be scanned using any method known to the skilled artisan [ref].

After acquiring an image of the array and saving the data, analyze results using information on the ProtoArray™ Application Portal to identify positive interactors.

ProtoArray™ Application Portal

The ProtoArray™ Application Portal provides a web-based user interface to retrieve ProtoArray™ Lot Specific information. This information is useful for analyzing the array data and identifying significant interactions.

If the scanner computer is connected to the internet, then click on the link below to connect to the portal. If the scanner computer is not connected to the internet, download the array-specific information as described below, burn the information on a CD, and then download the information onto the scanner computer.
1. Connect to the portal at www.invitrogen.com/protoarray and then click on the Online Tools tab.
2. A ProtoArray™ Lot Specific Information page is displayed.
3. Enter the array barcode in the Input Barcode Number box. Click on the Search button.

4. For each input barcode, 4 files are displayed as described below (see figure below). Array Image: GST array image for a Yeast ProtoArray™ PPI

| | |
|---|---|
| Array Image: | GST array image for a Yeast ProtoArray ™ |
| | PPI Control Microarray |
| .GAL file: | Defines spot locations and identities used |
| | by the microarray image analysis software |
| Protein Concentration | Description and concentration of protein |
| File: | spots on the array |
| Control Data File: | Description of control spots on the array |

5. Download 4 files listed above for array-specific information from a specific lot. Use these 4 files to interpret your results with the Yeast ProtoArray™ as described on the below.

Analyzing Data

It is strongly recommended to analyze the data using an image analysis software included with the fluorescent microplate scanner to identify significant interactions. Avoid identifying interactions visually.

There are multiple approaches to statistical analysis of microarray data. For detailed guidelines, refer to the ProtoArray™ Application Portal. A brief approach for data analysis is described below.
1. After acquiring the image and downloading the array-specific information from the ProtoArray™ Application Portal, use the image analysis software to localize spots, assign identities, and calculate corrected signal (signal/background) for each yeast protein spot.
2. Determine the median and standard deviation for the values and set a threshold to identify significant interactions. Note: We recommend using a value that is ≧3 standard deviations over the median value.

After identifying a significant interaction, be sure to:
Normalize the results for significant interactions using the protein concentration information from the ProtoArray™ Application Portal
Visually inspect the signal identified as significant
Verify that the interaction is a true positive interaction and not non-specific interactionby comparing the results with your probe to the negative control (an image of the negative control for the Yeast ProtoArray™ PPI Proteome Microarray is available on the ProtoArray™ Application Portal)
Check that the duplicate spot on the array gives similar results

After identifying a positive interaction using the Yeast ProtoArray™, you may validate the protein-protein interaction using methods such as:
Yeast Two-Hybrid Systems
Co-immunoprecipitation
Gel-shift assay
Determining reciprocal interactions with Yeast ProtoArray™

### EXAMPLE 2

### Method for Predicting a Biological Pathway

The following example illustrates the use of protein array data in combination with biological pathway knowledge and other data types, to generate biological pathway diagrams that indicate a predicted role of a test protein in a biological pathway. A protein array containing the majority of proteins from the yeast S. cerevisae was probed with a yeast protein phosphatase (Pph3). Two notable interactions were observed on the array. One interactor was identified as Tip41, a protein known to associate with Pph3 and a second interactor was identified as Rrd1 (Ito, T., et al., A comprehensive two-hybrid analysis to explore the yeast protein interactome. Proc Natl Acad Sci USA, 2001. 98(8): p. 4569-74). The function of Rrd1 is unknown but has been predicted to be a regulator of Pph3 because Pph3 overexpression can suppress the synthetic lethal phenotype of a rrd1,rrd2 double mutant (Rempola, B., et al., Functional analysis of RRD1 (Y1L153w) and RRD2 (YPL152w), which encode two putative activators of the phosphotyrosyl phosphatase activity of PP2A in Saccharomyces cerevisiae. Mol Gen Genet, 2000. 262(6): p. 1081-92 ). Therefore, the protein array data confirms the two-hybrid interactions of Tip41 with Pph3 and provides biochemical evidence implying that Rrd1 interacts directly with Pph3. Using software, a pathway diagram (FIG. 1) was built which depicts Rrd1 as an activator of Pph3, which in turn regulates cell cycle progression through the protein kinases Cla4, Swe1 and Cdc28 (Mitchell, D. A. and G. F. Sprague, Jr., The phosphotyrosyl phosphatase activator, Ncs1p (Rrd1p), functions with Cla4p to regulate the G(2)/M transition in Saccharomyces cerevisiae. Mol Cell Biol, 2001. 21(2): p. 488-500). While this example is relatively straightforward, the same basic approach can be employed with much larger datasets. Data from public or commercially available databases, for instance, can be integrated with the experimental data to create much more complex pathway diagrams with relatively little effort (for example, see Kyoto Encyclopedia of Genes and Genomes, http://www.genome.ad.jp/kegg/kegg2.html).

As shown in FIG. 1, multicomponent pathways can be postulated by integrating protein microarray data with other data types. In theory, it should also be possible to reconstitute pathways on protein arrays, an achievement recently accomplished with seven enzymes that carry out TreHalose synthesis (Jung, G. Y. and G. Stephanopoulos, A functional protein chip for pathway optimization and in vitro metabolic engineering. Science, 2004. 304(5669): p. 428-31). Because the contribution of each protein can be investigated by determining which proteins/enzymes are necessary for downstream signaling, protein microarrays can be used to both generate and test models of intracellular pathway signaling.

### REFERENCES CITED

Many modifications and variations of this invention can be made without departing from its spirit and scope, as will be apparent to those skilled in the art. The specific embodiments described herein are offered by way of example only, and the invention is to be limited only by the terms of the appended claims, along with the full scope of equivalents to which such claims are entitled. Such modifications are intended to fall within the scope of the appended claims.

All references, patent and non-patent, cited herein are incorporated herein by reference in their entireties and for all purposes to the same extent as if each individual publication or patent or patent application was specifically and individually indicated to be incorporated by reference in its entirety for all purposes.

Section headings included herein are for convenience only and are not intended to limited the invention, or to substantively affect this specification.

Provisional patent application entitled “Protein Arrays And Methods Of Use Thereof” filed on even date herewith is incorporated herein by reference in its entirety.

Further, the manual for the Yeast ProtoArray™ PPI kit, the ProtoArray™ Prospector v2.0 User Guide, and Schweitzer et al., Performance Characteristics of the Yeast ProtoArray™ Protein-Protein Interaction (PPI) Proteome Microarray, all found at the website of Invitrogen Corporation, are all incorporated herein by reference in their entireties.

1. A method for providing a protein microarray product to a customer, comprising:
a) providing the customer with access to a high density protein microarray product of a manufactured lot of protein microarray products, wherein the protein microarray product comprises at least 100 different proteins; and
b) providing the customer with protein identity and quantitative information regarding the at least 100 different proteins on each protein microarray product of the manufactured lot of protein microarray products, thereby providing the protein microarray product to the customer.

2-3. (canceled)

4. The method of claim 1, further comprising providing access to a computer function for obtaining the identity and quantitative information of proteins on the protein microarray product based on an identifier of the protein microarray product or the manufactured lot of protein microarray products.

5. The method of claim 4, wherein the computer function further provides access to the customer, to a purchasing function for identifying one or more target proteins on the protein microarray product, and for purchasing one or more related products and/or services related to the identified target proteins.

6. The method of claim 5, wherein the purchasing function presents the customer with access to a customized series of computer links to the related products and/or services based on the identified one or more target proteins.

7-9. (canceled)

10. The method of claim 6, wherein the computer function is an Internet portal that is provided over a wide area network to the customer by a provider of the protein microarray product.

11-65. (canceled)

66. A method for determining a strength of interaction between proteins on a protein microarray and a probe or a strength of enzymatic modification of proteins on the protein microarray by the probe, comprising
a) contacting proteins on the protein microarray with the probe to identify and quantify the strength of one or more positive signals on the microarray;
b) obtaining information regarding the identity and quantity of proteins on the protein microarray from a provider of the protein microarray;
c) identifying the proteins associated with the one or more positive signals using the information regarding the identity of proteins on the protein microarray; and
d) identifying the strength of interaction or enzymatic modification of the proteins on the protein microarray using the information regarding the identity and quantity of proteins on the protein microarray and the identity and strength of the positive signals.

67. (canceled)

68. A method according to claim 66, wherein the protein microarray comprises at least 100 different proteins.

69. A method according to claim 66, wherein the protein microarray comprises at least 1000 different proteins.

70. A method according to claim 66, wherein the protein microarray comprises recombinant yeast or mammalian proteins expressed in a eukaryotic host organism.

71. A method according to claim 66, wherein the quantitative information regarding proteins on the protein microarray comprises information regarding the concentration of proteins immobilized on the protein microarray.

72. A method according to claim 66, further comprising determining the concentration of the proteins on the protein microarray using a series of spots derived from solutions comprising different known concentrations of a tagged control protein.

73-80. (canceled)

81. A method for detecting labeling of a polypeptide, comprising:
a) labeling the polypeptide with a first specific binding pair member of a first binding pair;
b) analyzing the labeling by contacting the labeled polypeptide with a second specific binding pair member of the first binding pair, wherein the second specific binding pair member is associated with the surface of a control microarray; and
c) analyzing binding of the labeled polypeptide to the second specific binding pair member, wherein detectable binding of the labeled polypeptide to the second specific binding pair member associated with the control microarray is indicative of labeling of the polypeptide, thereby detecting labeling of the polypeptide.

82. The method of claim 81, further comprising analyzing the labeling of the polypeptide by a traditional method.

83-84. (canceled)

85. The method of claim 81, further comprising analyzing the quantity of labeling of the polypeptide using mass spectroscopy.

86-87. (canceled)

88. The method of claim 81, wherein the second specific binding pair member is an antibody that specifically binds to the label.

89. The method of claim 81, wherein the label comprises biotin or avidin.

90. (canceled)

91. The method of claim 81, wherein the control array further comprises a first specific binding pair member of a second binding pair, and the method further comprises contacting the control array with a second specific binding pair member of the second binding pair.

92-93. (canceled)

94. The method of claim 81, further comprising upon detectable binding of the labeled polypeptide to the second specific binding pair member associated with the surface of the control microarray, contacting a test microarray with the labeled polypeptide, wherein the test microarray is a protein microarray comprising 100 different proteins at a density of at least 100/cm².

95. A method according to claim 81, wherein the polypeptide is labeled in vivo.

96-125. (canceled)

126. A method for determining the concentration of a target protein in a solution, comprising:
a) providing a protein microarray comprising a spot of the target protein comprising a tag and a series of spots derived from solutions comprising different known concentrations of a tagged control protein;
b) contacting the protein microarray with a first specific binding pair member that binds the tag;
c) detecting a level of binding of the first specific binding pair member to the tag on the target polypeptide and to the different known concentrations of the tagged control protein; and
d) determining the concentration of the target protein using the level of binding of the first specific binding pair member to the tag on the target polypeptide and the level of binding of the first specific binding pair member to the different known concentrations of the tagged control protein, wherein the determining comprises a cubic curve fitting method.

127. The method of claim 126, wherein the tag is a protein purification tag.

128. The method of claim 126, wherein the protein purification tag is glutathione S-transferase.

129. The method of claim 126, wherein the microarray comprises a plurality of series of spots, each spot in a series comprising an identical tagged control protein, at least two series comprising different tags on the tagged control proteins.

130-148. (canceled)

149. A method for detecting labeling of a test polypeptide, comprising
a) labeling the polypeptide with a first specific binding pair member of a first binding pair;
b) contacting the labeled polypeptide with a control polypeptide on a control microarray, wherein the control polypeptide comprises a second specific binding pair member of the first binding pair; and
c) contacting the test microarray with the labeled polypeptide, wherein detectable binding of the labeled polypeptide to the second specific binding pair member of the first binding pair is indicative of labeling of the polypeptide, thereby detecting labeling of the test polypeptide.

150. The method of claim 149, further comprising analyzing the labeling of the polypeptide by a traditional method.

151-156. (canceled)

157. The method of claim 149, wherein the label is biotin or avidin.

158. The method of claim 157, wherein the polypeptide is labeled in vivo.

159. (canceled)

160. The method of claim 149, wherein the control array further comprises a first specific binding pair member of a second binding pair.

161. The method of claim 160, wherein the method further comprises contacting the control array with a second specific binding pair member of the second binding pair.

162. The method of claim 161, further comprising labeling the second specific binding pair member of the second specific binding pair with the first specific binding pair member of the first binding pair before contacting the control array with the second specific binding pair member of the second binding pair.
